(19) United States
(12) Patent Application Publication
(10) Pub. No.: US 2013/0270486 A1
(43) Pub. Date: Oct. 17, 2013

(54) POLYMER COMPOUND AND LIGHT-EMITTING DEVICE USING SAME

(71) Applicant: Tomoyasu Yoshida, Cambridgeshire (GB)

(72) Inventor: Tomoyasu Yoshida, Cambridgeshire (GB)

(73) Assignee: SUMITOMO CHEMICAL COMPANY, LIMITED, Chuo-ku, Tokyo (JP)

(21) Appl. No.: 13/995,195

(22) PCT Filed: Dec. 21, 2011
(86) PCT No.: PCT/JP2011/079591

§ 371 Date: Jun. 18, 2013

(30) Foreign Application Priority Data
Dec. 21, 2010 (JP) 2010-284957
Apr. 27, 2011 (JP) 2011-100018

(51) Int. Cl. H01L 51/00

(52) U.S. Cl. CPC H01L 51/0035
USPC 252/500; 526/239; 252/301.35; 570/183

(57) ABSTRACT

A polymer compound having a constitutional unit represented by the following formula (1) and a constitutional unit represented by the following formula (2):

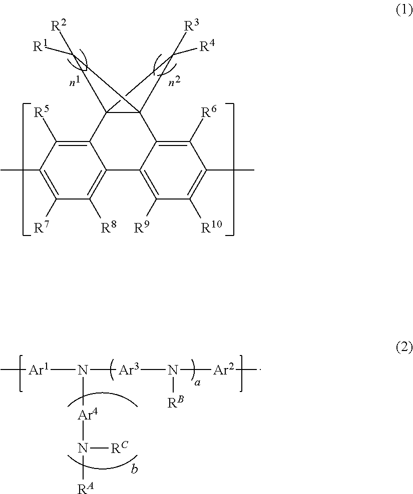

### TECHNICAL FIELD

The present invention relates to a polymer compound, its raw material compound, composition containing the polymer compound, a liquid composition containing the polymer compound, an organic film, a light-emitting device, and a display device.

### BACKGROUND ART

As a light-emitting material used for the light-emitting device, a polymer compound including a constitutional unit derived from arylamine (Patent Literature 1) and a polymer compound including a constitutional unit derived from fluorene (Patent Literature 2) have been examined, for example.

### CITATION LIST

### Patent Literature

Patent Literature 1: Japanese Patent Application Laid-Open No. 2004-143419
Patent Literature 2: National Publication of International Patent Application No. 2004-527628

### SUMMARY OF INVENTION

### Technical Problem

However, in the light-emitting device using the conventional polymer compound, its light emission efficiency is not always sufficient.

Then, the present invention is aimed to provide a polymer compound useful for production of a light-emitting device whose light emission efficiency is excellent. The present invention is moreover aimed to provide a composition containing the polymer compound, a liquid composition, an organic film, a light-emitting device, a surface light source, and a display device. The present invention is further aimed to provide a raw material compound for the polymer compound.

### Solution to Problem

The present invention provides a polymer compound having a constitutional unit represented by the following formula (1) and a constitutional unit represented by the following formula (2):

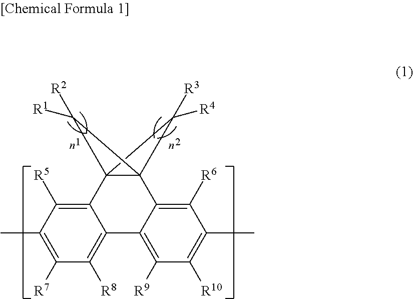

wherein n¹ and n² each independently represent an integer of 1 to 5; R¹, R², R³, R⁴, R⁵, R⁶, R⁷, R⁸, R⁹, and R¹⁰ each independently represent a hydrogen atom, an unsubstituted or substituted alkyl group, an unsubstituted or substituted alkoxy group, an unsubstituted or substituted aryl group, an unsubstituted or substituted aryloxy group, or an unsubstituted or substituted monovalent heterocyclic group; when R¹, R², R³, and R⁴ exist in plural, the plurality of R¹, R², R³, or R⁴ may be the same or different from each other; among R¹, R², R³, and R⁴, adjacent groups may be linked to each other to form a cyclic structure; and among R⁷, R⁸, R⁹, and R¹⁰, adjacent groups may be linked to each other to form a cyclic structure;

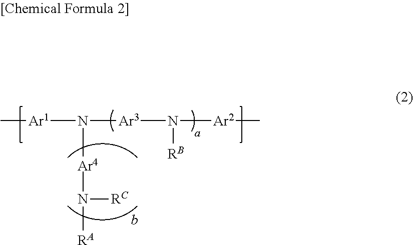

wherein a and b each independently represent 0 or 1; Ar¹, Ar², Ar³, and Ar⁴ each independently represent an unsubstituted or substituted arylene group, an unsubstituted or substituted divalent heterocyclic group, or a divalent group in which two or more same or different groups selected from an arylene group and divalent heterocyclic groups are linked (the divalent group may have a substituent); R^{A}, R^{B}, and R^{C} each independently represent a hydrogen atom, an unsubstituted or substituted alkyl group, an unsubstituted or substituted aryl group, or an unsubstituted or substituted monovalent heterocyclic group; and Ar¹, Ar², Ar³, and Ar⁴ each may be bonded to a group other than the group to form a cyclic structure, the other group being bonded to a nitrogen atom to which the group is bonded.

According to such a polymer compound, a light-emitting device whose light emission efficiency is excellent is obtained.

The polymer compound according to the present invention may have a constitutional unit represented by the following formula (3) as the constitutional unit represented by the above formula (2):

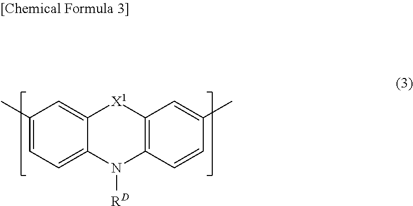

wherein R^{D} represents a hydrogen atom, an unsubstituted or substituted alkyl group, an unsubstituted or substituted aryl group, or an unsubstituted or substituted monovalent heterocyclic group; and X¹ represents a single bond, an oxygen atom, a sulfur atom, or a group represented by —C(R¹¹)₂— (R¹¹ represents an unsubstituted or substituted alkyl group or an unsubstituted or substituted aryl group; and a plurality of R¹¹ present may be the same or different from each other).

The polymer compound according to the present invention may further have a constitutional unit represented by the following formula (4):

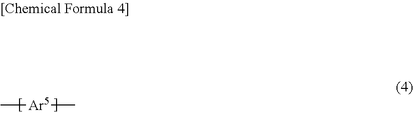

wherein Ar⁵ represents an unsubstituted or substituted arylene group, an unsubstituted or substituted divalent heterocyclic group, or a divalent group in which two or more same or different groups selected from arylene groups and divalent heterocyclic groups are linked (the divalent group may have a substituent); and the constitutional unit represented by the formula (4) is different from the constitutional unit represented by the formula (1).

The polymer compound according to the present invention may have a constitutional unit consisting of an unsubstituted or substituted fluorenediyl group as the constitutional unit represented by the above formula (4).

The polymer compound according to the present invention may have a constitutional unit consisting of an unsubstituted or substituted 2,7-fluorenediyl group as the constitutional unit represented by the above formula (4).

The polymer compound according to the present invention may have a constitutional unit consisting of the group selected from the group consisting of an unsubstituted or substituted phenylene group, an unsubstituted or substituted naphthalenediyl group, an unsubstituted or substituted anthracenediyl group, and a group represented by the following formula (5′) as the constitutional unit represented by the above formula (4):

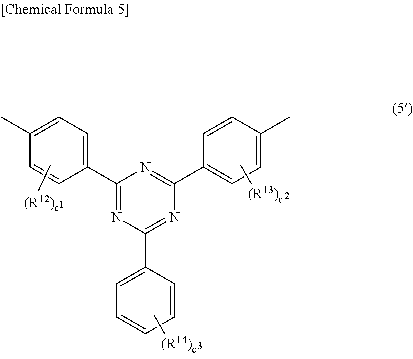

wherein c¹ and c² each independently represent an integer of 0 to 4; c³ represents an integer of 0 to 5; R¹², R¹³, and R¹⁴ each independently represent an unsubstituted or substituted alkyl group, an unsubstituted or substituted alkoxy group, an unsubstituted or substituted aryl group, an unsubstituted or substituted aryloxy group, an unsubstituted or substituted monovalent heterocyclic group, an unsubstituted or substituted alkoxycarbonyl group, an unsubstituted or substituted silyl group, a halogen atom, a carboxyl group, or a cyano group; and when R¹², R¹³, and R¹⁴ exist in plural, the plurality of R¹², R¹³, or R¹⁴ may be the same or different from each other.

The polymer compound according to the present invention may have the constitutional unit represented by the above formula (1), the constitutional unit represented by the above formula (2), a constitutional unit consisting of an unsubstituted or substituted fluorenediyl group, and a constitutional unit consisting of an unsubstituted or substituted phenylene group.

The polymer compound according to the present invention may have the constitutional unit represented by the above formula (1), the constitutional unit represented by the above formula (2), a constitutional unit consisting of an unsubstituted or substituted fluorenediyl group, and a constitutional unit consisting of an unsubstituted or substituted naphthalenediyl group.

The polymer compound according to the present invention may have the constitutional unit represented by the above formula (1), the constitutional unit represented by the above formula (2), a constitutional unit consisting of an unsubstituted or substituted fluorenediyl group, and a constitutional unit consisting of an unsubstituted or substituted anthracenediyl group.

The polymer compound according to the present invention may have the constitutional unit represented by the above formula (1), the constitutional unit represented by the above formula (2), a constitutional unit consisting of an unsubstituted or substituted fluorenediyl group, and the constitutional unit represented by the following formula (5) (namely, the constitutional unit consisting of the group represented by the formula (5′)):

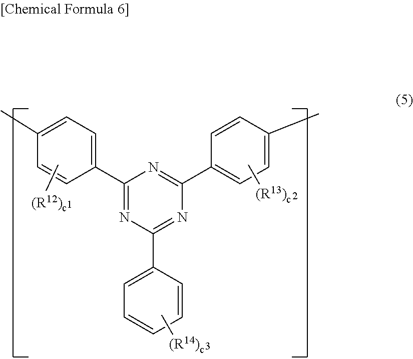

wherein c¹ and c² each independently represent an integer of 0 to 4; c³ represents an integer of 0 to 5; R¹², R¹³, and R¹⁴ each independently represent an unsubstituted or substituted alkyl group, an unsubstituted or substituted alkoxy group, an unsubstituted or substituted aryl group, an unsubstituted or substituted aryloxy group, an unsubstituted or substituted monovalent heterocyclic group, an unsubstituted or substituted alkoxycarbonyl group, an unsubstituted or substituted silyl group, a halogen atom, a carboxyl group, or a cyano group; and when R¹², R¹³, and R¹⁴ exist in plural, the plurality of R¹², R¹³, or R¹⁴ may be the same or different from each other.

In the polymer compound according to the present invention, n¹ and n² in the above formula (1) each independently may be 3 or 4.

Moreover, the present invention provides a compound represented by the following formula (6):

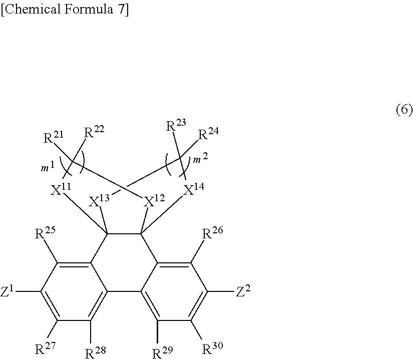

wherein m¹ and m² each independently represent 1 or 2; R²¹, R²², R²³, R²⁴, R²⁵, R²⁶, R²⁷, R²⁸, R²⁹, and R³⁰ each independently represent a hydrogen atom, an unsubstituted or substituted alkyl group, an unsubstituted or substituted alkoxy group, an unsubstituted or substituted aryl group, an unsubstituted or substituted aryloxy group, or an unsubstituted or substituted monovalent heterocyclic group; X¹¹, X¹², X¹³, and X¹⁴ each independently represent a group represented by —C(R³¹)₂— (R³¹ represents a hydrogen atom, an unsubstituted or substituted alkyl group, an unsubstituted or substituted alkoxy group, an unsubstituted or substituted aryl group, an unsubstituted or substituted aryloxy group, or an unsubstituted or substituted monovalent heterocyclic group; and a plurality of R³¹ present may be the same or different from each other); among R²⁷, R²⁸, R²⁹, and R³⁰, adjacent groups may be linked to each other to form a cyclic structure; and Z¹ and Z² each independently represent a group selected from the R²², R²³, following substituent group; with the proviso that among R²¹, R²², R²³, and R²⁴, at least one is a group other than a hydrogen atom;

### <Substituent Group>

a chlorine atom, a bromine atom, iodine atom,

a group represented by —O—S(═O)₂R⁴¹ wherein R⁴¹ represents an alkyl group, or an aryl group that may be substituted with an alkyl group, an alkoxy group, a nitro group, a fluorine atom, or a cyano group,

a group represented by —B(OR⁴²)₂ wherein R⁴² represents a hydrogen atom or an alkyl group; and a plurality of R⁴² present may be the same or different from each other and may be bonded to each other to form a cyclic structure,

a group represented by —BF₄Q¹ wherein Q¹ represents a monovalent cation selected from the group consisting of Li⁺, Na⁺, K⁺, Rb⁺, and Cs⁺,

a group represented by —MgY¹ wherein Y¹ represents a chlorine atom, a bromine atom, or an iodine atom,

a group represented by —ZnY² wherein Y² represents a chlorine atom, a bromine atom, or an iodine atom, and

a group represented by —Sn(R⁴³)₃ wherein R⁴³ represents a hydrogen atom or an alkyl group; and a plurality of R⁴³ present may be the same or different from each other and may be bonded to each other to form a cyclic structure.

Moreover, the present invention provides a composition containing the polymer compound according to the present invention and at least one selected from the group consisting of a hole transport material, an electron transport material, and a light-emitting material. Such a composition can be suitably used in production of the light-emitting device, and the light-emitting device to be obtained is excellent in light emission efficiency.

Moreover, the present invention provides a liquid composition containing the polymer compound according to the present invention and a solvent. According to such a liquid composition, an organic film containing the polymer compound can be easily produced.

Moreover, the present invention provides an organic film containing the polymer compound according to the present invention. Such an organic film is useful for production of the light-emitting device whose light emission efficiency is excellent.

Moreover, the present invention provides an organic film using the composition according to the present invention. Such an organic film is useful for production of the light-emitting device whose light emission efficiency is excellent.

Moreover, the present invention provides a light-emitting device having the organic film according to the present invention. Such a light-emitting device is excellent in light emission efficiency.

Moreover, the present invention provides a surface light source and a display device having the light-emitting device according to the present invention.

### Advantageous Effects of Invention

According to the present invention, a polymer compound useful for production of a light-emitting device whose light emission efficiency is excellent can be provided. Moreover, according to the present invention, a composition, liquid composition, organic film, light-emitting device, surface light source, and display device containing the polymer compound can be provided. Further, according to the present invention, a raw material compound for the polymer compound can be provided.

### BRIEF DESCRIPTION OF DRAWINGS

FIG. 1 is a schematic sectional view showing one embodiment of a light-emitting device according to the present invention.

FIG. 2 is a schematic sectional view showing another embodiment of a light-emitting device according to the present invention.

FIG. 3 is a schematic sectional view showing one embodiment of a surface light source according to the present invention.

### DESCRIPTION OF EMBODIMENTS

Hereinafter, terms commonly used herein will be described, using examples when necessary.

Herein, “Me” represents a methyl group, “Et” represents an ethyl group, “Ph” represents a phenyl group, and “t-Bu” represents a tert-butyl group.

The “constitutional unit” means one or more unit structures that are present in the polymer compound. It is preferable that the “constitutional unit” be included in the polymer compound as a “repeating unit” (namely, two or more unit structures that are present in the polymer compound).

The term “C_{X} to C_{y}” (x and y are a positive integer that satisfies x<y) means that the number of carbon atoms in a partial structure corresponding to the name of the functional group written immediately after the term is x to y. Namely, in the case where the organic group written immediately after “C_{X} to C_{y}” is an organic group named in combination of a plurality of names of functional groups (for example, a C_{X} to C_{y} alkoxyphenyl group), the term means that among the plurality of names of functional groups, the number of carbon atoms in the partial structure corresponding to the name of the functional group written immediately after “C_{X} to C_{y}” (for example, alkoxy) is x to y. For example, the “C₁ to C₁₂ alkyl group” means an alkyl group having 1 to 12 carbon atoms, and the “C₁ to C₁₂ alkoxyphenyl group” means a phenyl group having an “alkoxy group having 1 to 12 carbon atoms.”

Herein, the term “unsubstituted or substituted” means that the functional group written immediately after the term may have a substituent. For example, the “unsubstituted or substituted alkyl group” means an “unsubstituted alkyl group or an alkyl group having a substituent.”

Examples of the substituent include an alkyl group, an alkoxy group, an alkylthio group, an aryl group, an aryloxy group, an arylthio group, an alkenyl group, an alkynyl group, an amino group, a silyl group, halogen atoms, an acyl group, an acyloxy group, an oxycarbonyl group, a monovalent heterocyclic group, a heterocycleoxy group, a heterocyclethio group, imine residues, amide compound residues, acid imide residues, a carboxyl group, a hydroxy group, a nitro group, and a cyano group. These groups may further have a substituent selected from the groups above.

The “alkyl group” may have a substituent, and may be any of a linear alkyl group, a branched alkyl group, and a cyclic alkyl group (cycloalkyl group). Unless otherwise specified, without including the number of carbon atoms of the substituent, the number of carbon atoms of the alkyl group is preferably 1 to 20, more preferably 1 to 15, and still more preferably 1 to 12 in the linear alkyl group and the branched alkyl group; without including the number of carbon atoms of the substituent, the number of carbon atoms of the alkyl group is preferably 3 to 20, more preferably 3 to 15, and still more preferably 3 to 12 in the cyclic alkyl group. Examples of the alkyl group include a methyl group, an ethyl group, a propyl group, an isopropyl group, a butyl group, an isobutyl group, a sec-butyl group, a tert-butyl group, a pentyl group, an isoamyl group, a hexyl group, a cyclohexyl group, a heptyl group, an octyl group, a 2-ethylhexyl group, a nonyl group, a decyl group, a 3,7-dimethyloctyl group, and a dodecyl group.

The “alkoxy group” may have a substituent, and may be any of a linear alkoxy group, a branched alkoxy group, and a cyclic alkoxy group (cycloalkoxy group). Unless otherwise specified, without including the number of carbon atoms of the substituent, the number of carbon atoms of the alkoxy group is preferably 1 to 20, more preferably 1 to 15, and still more preferably 1 to 12 in the linear alkoxy group and the branched alkoxy group; without including the number of carbon atoms of the substituent, the number of carbon atoms of the alkoxy group is preferably 3 to 20, more preferably 3 to 15, and still more preferably 3 to 12 in the cyclic alkoxy group. Examples of the alkoxy group include a methoxy group, ethoxy group, a propyloxy group, an isopropyloxy group, a butoxy group, an isobutoxy group, a sec-butoxy group, a tert-butoxy group, a pentyloxy group, a hexyloxy group, a cyclohexyloxy group, a heptyloxy group, an octyloxy group, a 2-ethylhexyloxy group, a nonyloxy group, a decyloxy group, a 3,7-dimethyloctyloxy group, and a dodecyloxy group.

The “alkylthio group” may have a substituent, and may be any of a linear alkylthio group, a branched alkylthio group, and a cyclic alkylthio group (cycloalkylthio group). Unless otherwise specified, without including the number of carbon atoms of the substituent, the number of carbon atoms of the alkoxy group is preferably 1 to 20, more preferably 1 to 15, and still more preferably 1 to 12 in the linear alkylthio group and the branched alkylthio group; without including the number of carbon atoms of the substituent, the number of carbon atoms of the alkoxy group is preferably 3 to 20, more preferably 3 to 15, and still more preferably 3 to 12 in the cyclic alkylthio group. Examples of the alkylthio group include a methylthio group, an ethylthio group, a propylthio group, an isopropylthio group, a butylthio group, an isobutylthio group, a sec-butylthio group, a tert-butylthio group, a pentylthio group, a hexylthio group, a cyclohexylthio group, a heptylthio group, an octylthio group, a 2-ethylhexylthio group, a nonylthio group, a decylthio group, a 3,7-dimethyloctylthio group, and a dodecylthio group.

The “aryl group” is the remaining atomic group in which one hydrogen atom bonded to carbon atoms that form an aromatic ring is removed from an aromatic hydrocarbon. The aryl group may have a substituent, and examples of the aryl group include those having a benzene ring, and those having a condensation ring. Unless otherwise specified, without including the number of carbon atoms of the substituent, the number of carbon atoms of the aryl group is preferably 6 to 60, more preferably 6 to 48, and still more preferably 6 to 30. Examples of the aromatic hydrocarbon include benzene, naphthalene, anthracene, phenanthrene, naphthacene, fluorene, pyrene, and perylene. Examples of the aryl group include a phenyl group, a 1-naphthyl group, a 2-naphthyl group, a 1-anthracenyl group, a 2-anthracenyl group, a 9-anthracenyl group, and 2-fluorenyl group.

The “aryloxy group” is the group represented by —O—Ar¹¹ (Ar¹¹ represents the aryl group), and the aryl group in Ar¹¹ may have a substituent. Unless otherwise specified, without including the number of carbon atoms of the substituent, the number of carbon atoms of the aryloxy group is preferably 6 to 60, more preferably 6 to 48, and still more preferably 6 to 30. Examples of the aryloxy group include a phenoxy group, a 1-naphthyloxy group, a 2-naphthyloxy group, a 1-anthracenyloxy group, a 2-anthracenyloxy group, a 9-anthracenyloxy group, and a 2-fluorenyloxy group.

The “arylthio group” is the group represented by —S—Ar¹² (Ar¹² represents the aryl group), and the aryl group in Ar¹² may have a substituent. Unless otherwise specified, without including the number of carbon atoms of the substituent, the number of carbon atoms of the arylthio group is preferably 6 to 60, more preferably 6 to 48, and still more preferably 6 to 30. Examples of the arylthio group include a phenylthio group, a 1-naphthylthio group, a 2-naphthylthio group, a 1-anthracenylthio group, a 2-anthracenylthio group, a 9-anthracenylthio group, and a 2-fluorenylthio group.

The “alkenyl group” is the remaining atomic group in which one hydrogen atom bonded to sp² carbons in alkene is removed. The alkenyl group may have a substituent, and may be any of a linear alkenyl group, a branched alkenyl group, and a cyclic alkenyl group. Unless otherwise specified, without including the number of carbon atoms of the substituent, the number of carbon atoms of the alkenyl group is preferably 2 to 20, more preferably 2 to 15, and still more preferably 2 to 10 in the linear alkenyl group and the branched alkenyl group; without including the number of carbon atoms of the substituent, the number of carbon atoms of the alkenyl group is preferably 3 to 20, more preferably 4 to 15, and still more preferably 5 to 10 in the cyclic alkenyl group. Examples of the alkenyl group include a vinyl group, a 1-propenyl group, a 2-propenyl group, a 1-butenyl group, a 2-butenyl group, a 1-pentenyl group, a 2-pentenyl group, a 1-hexenyl group, a 2-hexenyl group, and a 1-octenyl group.

The “alkynyl group” is the remaining atomic group in which one hydrogen atom bonded to sp¹ carbons in alkyne is removed. The alkynyl group may have a substituent, and may be any of a linear alkynyl group, a branched alkynyl group, and a cyclic alkynyl group. Unless otherwise specified, without including the number of carbon atoms of the substituent, the number of carbon atoms of the alkynyl group is preferably 2 to 20, more preferably 2 to 15, and still more preferably 2 to 10 in the linear alkynyl group and the branched alkynyl group; without including the number of carbon atoms of the substituent, the number of carbon atoms of the alkynyl group is preferably 5 to 20, more preferably 6 to 15, and still more preferably 7 to 10 in the cyclic alkynyl group. Examples of the alkynyl group include an ethynyl group, a 1-propynyl group, a 2-propynyl group, a 1-butynyl group, a 2-butynyl group, a 1-pentynyl group, a 2-pentynyl group, a 1-hexynyl group, a 2-hexynyl group, and a 1-octynyl group.

The “amino group” may have a substituent, and is preferably an unsubstituted amino group and an amino group substituted with one or two substituents selected from an alkyl group, an aryl group, an arylalkyl group, and a monovalent heterocyclic group (hereinafter, referred to as a “substituted amino group”). The substituent may further have a substituent (hereinafter, a substituent that a substituent having an organic group further has is referred to as a “secondary substituent” in some cases). Without including the number of carbon atoms of the secondary substituent, the number of carbon atoms of the substituted amino group is preferably 1 to 60, more preferably 2 to 48, and still more preferably 2 to 40.

Examples of the substituted amino group include a methylamino group, a dimethylamino group, an ethylamino group, a diethylamino group, a propylamino group, a dipropylamino group, an isopropylamino group, a diisopropylamino group, a butylamino group, an isobutylamino group, a sec-butylamino group, a tert-butylamino group, a pentylamino group, a hexylamino group, a heptylamino group, an octylamino group, a 2-ethylhexylamino group, a nonylamino group, a decylamino group, a 3,7-dimethyloctylamino group, a dodecylamino group, a cyclopentylamino group, a dicyclopentylamino group, a cyclohexylamino group, a dicyclohexylamino group, a ditrifluoromethylamino group, a phenylamino group, a diphenylamino group, a C₁ to C₁₂ alkoxyphenylamino group, a bis(C₁ to C₁₂ alkoxyphenyl)amino group, a C₁ to C₁₂ alkylphenylamino group, a bis(C₁ to C₁₂ alkylphenyl)amino group, a 1-naphthylamino group, a 2-naphthylamino group, a pentafluorophenylamino group, a pyridylamino group, a pyridazinylamino group, a pyrimidinylamino group, a pyrazinylamino group, a triazinylamino group, a phenyl-C₁ to C₁₂ alkylamino group, a C₁ to C₁₂ alkoxyphenyl-C₁ to C₁₂ alkylamino group, a di(C₁ to C₁₂ alkoxyphenyl-C₁ to C₁₂ alkyl)amino group, a C₁ to C₁₂ alkylphenyl-C₁ to C₁₂ alkylamino group, a di(C₁ to C₁₂ alkylphenyl-C₁ to C₁₂ alkyl)amino group, a l-naphthyl-C₁ to C₁₂ alkylamino group, and a 2-naphthyl-C₁ to C₁₂ alkylamino group.

The “silyl group” may have a substituent, and is preferably an unsubstituted silyl group and a silyl group substituted with one to three substituents selected from an alkyl group, an aryl group, an arylalkyl group, and a monovalent heterocyclic group (hereinafter, referred to as a “substituted silyl group”). The substituent may have a secondary substituent. Without including the number of carbon atoms of the secondary substituent, the number of carbon atoms of the substituted silyl group is preferably 1 to 60, more preferably 3 to 48, and still more preferably 3 to 40.

Examples of the substituted silyl group include a trimethylsilyl group, a triethylsilyl group, a tripropylsilyl group, a tri-isopropylsilyl group, a dimethyl-isopropylsilyl group, a diethyl-isopropylsilyl group, a tert-butyldimethylsilyl group, a pentyldimethylsilyl group, a hexyldimethylsilyl group, a heptyldimethylsilyl group, an octyldimethylsilyl group, a 2-ethylhexyl-dimethylsilyl group, a nonyldimethylsilyl group, a decyldimethylsilyl group, a 3,7-dimethyloctyl-dimethylsilyl group, a dodecyldimethylsilyl group, a phenyl-C₁ to C₁₂ alkylsilyl group, a C₁ to C₁₂ alkoxyphenyl-C₁ to C₁₂ alkylsilyl group, a C₁ to C₁₂ alkylphenyl-C₁ to C₁₂ alkylsilyl group, a 1-naphthyl-C₁ to C₁₂ alkylsilyl group, a 2-naphthyl-C₁ to C₁₂ alkylsilyl group, a phenyl-C₁ to C₁₂ alkyldimethylsilyl group, a triphenylsilyl group, a tri-p-xylylsilyl group, a tribenzylsilyl group, a diphenylmethylsilyl group, a tert-butyldiphenylsilyl group, and a dimethylphenylsilyl group.

Examples of the “acyl group” include groups represented by —C(═O)—R⁴⁴ (R⁴⁴ represents the alkyl group, the aryl group, or a monovalent heterocyclic group described later). The alkyl group, the aryl group, and the monovalent heterocyclic group in R⁴⁴ may have a substituent. Unless otherwise specified, without including the number of carbon atoms of the substituent, the number of carbon atoms of the acyl group is preferably 2 to 20, more preferably 2 to 18, and still more preferably 2 to 16. Examples of the acyl group include an acetyl group, a propionyl group, a butyryl group, an isobutyryl group, a pivaloyl group, and a benzoyl group. Examples of the acyl group having a substituent include an acyl group having a halogen atom as a substituent (such as a trifluoroacetyl group and a pentafluorobenzoyl group).

Examples of the “acyloxy group” include groups represented by —O—C(═O)—R⁴⁵ (R⁴⁵ represents the alkyl group, the aryl group, or a monovalent heterocyclic group described later). The alkyl group, the aryl group, and the monovalent heterocyclic group in R⁴⁵ may have a substituent. Unless otherwise specified, without including the number of carbon atoms of the substituent, the number of carbon atoms of the acyloxy group is preferably 2 to 20, more preferably 2 to 18, and still more preferably 2 to 16. Examples of the acyloxy group include an acetoxy group, a propionyloxy group, a butyryloxy group, an isobutyryloxy group, a pivaloyloxy group, and a benzoyloxy group. Examples of the acyloxy group having a substituent include an acyloxy group having a halogen atom as a substituent (such as a trifluoroacetyloxy group and a pentafluorobenzoyloxy group).

Examples of the “oxycarbonyl group” include groups represented by —C(═O)—O—R^{45a} (R^{45a} represents the alkyl group, the aryl group, or a monovalent heterocyclic group described later). The alkyl group, the aryl group, and the monovalent heterocyclic group in R^{45a} may have a substituent. Unless otherwise specified, without including the number of carbon atoms of the substituent, the number of carbon atoms of the oxycarbonyl group is preferably 2 to 20, more preferably 2 to 18, and still more preferably 2 to 16.

The “monovalent heterocyclic group” is the remaining atomic group in which one hydrogen atom is removed from a heterocyclic compound. The heterocyclic group may have a substituent, and examples of the heterocyclic group include a monocyclic group, and a group having a condensation ring. Unless otherwise specified, without including the number of carbon atoms of the substituent, the number of carbon atoms in the monovalent heterocyclic group is preferably 4 to 60, more preferably 4 to 30, and still more preferably 4 to 20.

The heterocyclic compound designates compounds among organic compounds having a cyclic structure and the compounds including not only a carbon atom but also a hetero atom such as an oxygen atom, a sulfur atom, a nitrogen atom, a phosphorus atom, a boron atom, a silicon atom, a selenium atom, a tellurium atom, and an arsenic atom as the element that forms the ring.

As the monovalent heterocyclic group, monovalent aromatic heterocyclic groups are preferable. The monovalent aromatic heterocyclic group is the remaining atomic group in which one hydrogen atom is removed from an aromatic heterocyclic compound. Examples of the aromatic heterocyclic compound include compounds in which a heterocyclic ring itself containing a hetero atom demonstrates aromaticity, such as oxadiazole, thiadiazole, thiazole, oxazole, thiophene, pyrrole, phosphole, furan, pyridine, pyrazine, pyrimidine, triazine, pyridazin, quinoline, isoquinoline, carbazole, dibenzophosphole, dibenzofuran, and dibenzothiophene; and compounds in which a heterocyclic ring itself containing a hetero atom does not demonstrate aromaticity, but an aromatic ring is fused to the heterocycle, such as phenoxazine, phenothiazine, dibenzoborole, dibenzosilole, and benzopyran.

The “heterocycleoxy group” is a group represented by —O—Ar¹³ (Ar¹³ represents the monovalent heterocyclic group), and the heterocyclic group in Ar¹³ may have a substituent. Unless otherwise specified, without including the number of carbon atoms of the substituent, the number of carbon atoms of the heterocycleoxy group is preferably 4 to 60, more preferably 4 to 30, and still more preferably 4 to 20. Examples of the heterocycleoxy group include a pyridyloxy group, a pyridazinyloxy group, a pyrimidinyloxy group, a pyrazinyloxy group, and a triazinyloxy group.

The “heterocyclethio group” is a group represented by —S—Ar¹⁴ (Ar¹⁴ represents the monovalent heterocyclic group), and the heterocyclic group in Ar¹⁴ may have a substituent. Unless otherwise specified, without including the number of carbon atoms of the substituent, the number of carbon atoms of the heterocyclethio group is preferably 4 to 60, more preferably 4 to 30, and still more preferably 4 to 20. Examples of the heterocyclethio group include a pyridylthio group, a pyridazinylthio group, a pyrimidinylthio group, a pyrazinylthio group, and a triazinylthio group.

The “imine residue” means a residue in which a hydrogen atom in the formula is removed from an imine compound having a structure represented by at least one of the formula: H—N═C(R⁴⁶)₂ and the formula: H—C(R⁴⁷)═N—R⁴⁸. In the formulas, R⁴⁶, R⁴⁷, and R⁴⁸ each independently represent the alkyl group, the aryl group, the alkenyl group, the alkynyl group, or the monovalent heterocyclic group. The alkyl group, the aryl group, the alkenyl group, the alkynyl group, and the monovalent heterocyclic group in R⁴⁶, R⁴⁷ and R⁴⁸ may have a substituent. A plurality of R⁴⁶ present may be the same or different from each other, or may be linked to each other to form a cyclic structure. Examples of the imine residue include groups represented by the following structure:

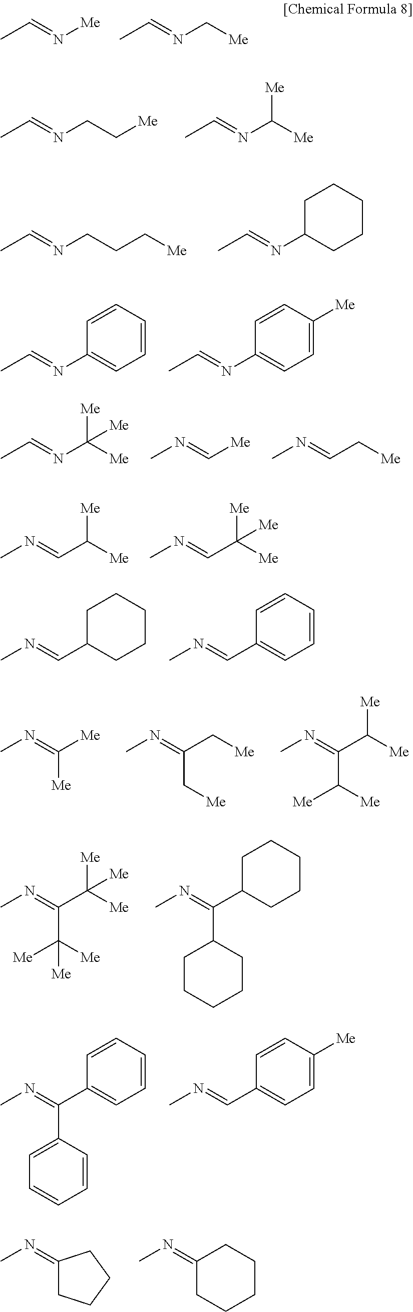

The “amide compound residue” means a residue in which a hydrogen atom in the formula is removed from an amide compound having a structure represented by at least one of the formula: H—N(R⁴⁹)—C(═O)R⁵ and the formula: H—C(═O)—N(R⁵¹)₂. In the formulas, R⁴⁹, R⁵⁰, and R⁵¹ each independently represent the alkyl group, the aryl group, the alkenyl group, the alkynyl group, or the monovalent heterocyclic group. The alkyl group, the aryl group, the alkenyl group, the alkynyl group, and the monovalent heterocyclic group in R⁴⁹, R⁵⁰, and R⁵¹ may have a substituent. A plurality of R⁵¹ present may be the same or different from each other, and may be linked to each other to form a cyclic structure. Examples of the amide compound residue include formamide residues, acetoamide residues, propioamide residues, butyroamide residues, benzamide residues, trifluoroacetoamide residues, pentafluorobenzamide residues, diformamide residues, diacetoamide residues, dipropioamide residues, dibutyroamide residues, dibenzamide residues, ditrifluoroacetoamide residues, and dipentafluorobenzamide residues.

The “acid imide residue” means a residue obtained by removing one hydrogen atom bonded to a nitrogen atom from an acid imide. The number of carbon atoms of the acid imide residue is preferably 4 to 20, more preferably 4 to 18, and still more preferably 4 to 16. Examples of the acid imide residue include groups represented by the following structure:

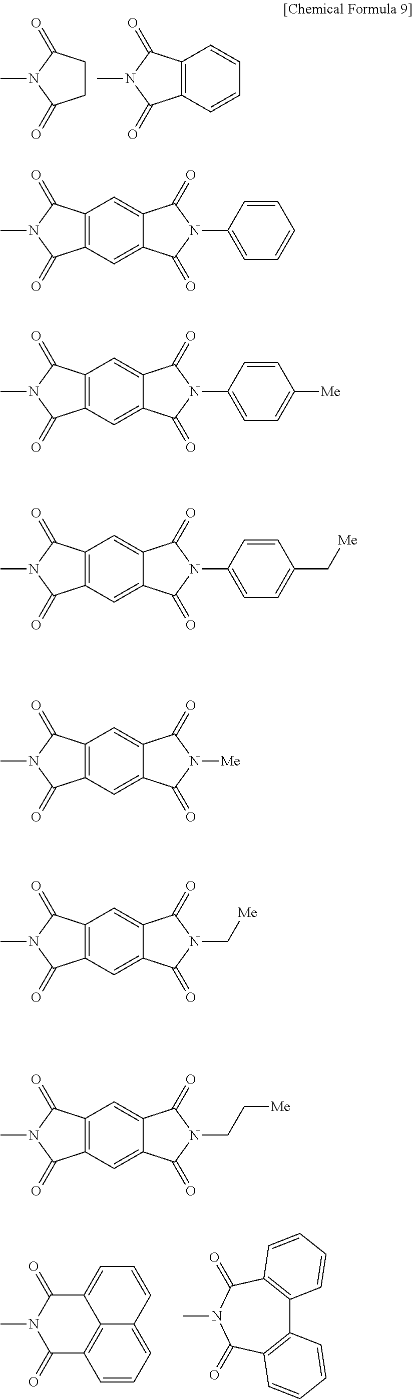

Examples of the “unsubstituted or substituted alkyl group” include unsubstituted alkyl groups and the alkyl groups having substituents above. Here, the substituent that the alkyl group has is preferably a substituent selected from an alkoxy group, an aryl group, an aryloxy group, a monovalent heterocyclic group, a heterocycleoxy group, and a halogen atom, unless otherwise specified.

Examples of the “unsubstituted or substituted alkoxy group” include unsubstituted alkoxy groups and the alkoxy groups having substituents above. Here, the substituent that the alkoxy group has is preferably a substituent selected from an alkoxy group, an aryl group, an aryloxy group, a monovalent heterocyclic group, a heterocycleoxy group, and a halogen atom, unless otherwise specified.

Examples of the “unsubstituted or substituted aryl group” include unsubstituted aryl groups and the aryl groups having the substituents above. Here, the substituent that the aryl group has is preferably a substituent selected from an alkyl group, an alkoxy group, an aryl group, an aryloxy group, a monovalent heterocyclic group, a heterocycleoxy group, and a halogen atom unless otherwise specified.

Examples of the “unsubstituted or substituted aryloxy group” include unsubstituted aryloxy groups and aryloxy groups having the substituents above. Here, the substituent that the aryloxy group has is preferably a substituent selected from an alkyl group, an alkoxy group, an aryl group, an aryloxy group, a monovalent heterocyclic group, a heterocycleoxy group, and a halogen atom unless otherwise specified.

Examples of the “unsubstituted or substituted monovalent heterocyclic group” include unsubstituted monovalent heterocyclic groups and monovalent heterocyclic groups having the substituents above. Here, the substituent that the monovalent heterocyclic group has is preferably a substituent selected from an alkyl group, an alkoxy group, an aryl group, an aryloxy group, a monovalent heterocyclic group, a heterocycleoxy group, and a halogen atom unless otherwise specified.

Examples of the “unsubstituted or substituted arylene group” include unsubstituted arylene groups and arylene groups having the substituents above. Here, the substituent that the arylene group has is preferably a substituent selected from an alkyl group, an alkoxy group, an aryl group, an aryloxy group, a monovalent heterocyclic group, a heterocycleoxy group and a halogen atom unless otherwise specified.

The “arylene group” is the remaining atomic group in which two hydrogen atoms bonded to carbon atoms that form an aromatic ring are removed from an aromatic hydrocarbon. The arylene group may have a substituent, and groups having a benzene ring and groups having a condensation ring are included in the arylene group. Unless otherwise specified, without including the number of carbon atoms of the substituent, the number of carbon atoms of the arylene group is preferably 6 to 60, more preferably 6 to 48, and still more preferably 6 to 30.

Examples of the aromatic hydrocarbon include benzene, naphthalene, anthracene, phenanthrene, naphthacene, fluorene, pyrene, and perylene. Examples of the arylene group include phenylene groups such as a 1,4-phenylene group, a 1,3-phenylene group, and a 1,2-phenylene group; naphthalenediyl groups such as a 1,4-naphthalenediyl group, a 1,5-naphthalenediyl group, 2,6-naphthalenediyl group, and a 2,7-naphthalenediyl group; anthracenediyl groups such as a 1,4-anthracenediyl group, a 1,5-anthracenediyl group, a 2,6-anthracenediyl group, and a 9,10-anthracenediyl group; phenanthrenediyl groups such as a 2,7-phenanthrenediyl group; naphthacenediyl groups such as a 1,7-naphthacenediyl group, a 2,8-naphthacenediyl group, and a 5,12-naphthacenediyl group; fluorenediyl groups such as a 2,7-fluorenediyl group and a 3,6-fluorenediyl group; pyrenediyl groups such as a 1,6-pyrenediyl group, a 1,8-pyrenediyl group, a 2,7-pyrenediyl group, and a 4,9-pyrenediyl group; and perylenediyl groups such as a 3,8-perylenediyl group, a 3,9-perylenediyl group, and a 3,10-perylenediyl group.

Examples of the “unsubstituted or substituted divalent heterocyclic group” include unsubstituted divalent heterocyclic groups and divalent heterocyclic groups having the substituents above. Here, the substituent that the divalent heterocyclic group has is preferably a substituent selected from an alkyl group, an alkoxy group, an aryl group, an aryloxy group, a monovalent heterocyclic group, a heterocycleoxy group, and a halogen atom unless otherwise specified.

The “divalent heterocyclic group” is the remaining atomic group in which two hydrogen atoms are removed from a heterocyclic compound. The divalent heterocyclic group may have a substituent, and monocyclic groups and groups having a condensation ring are included in the divalent heterocyclic group. Unless otherwise specified, without including the number of carbon atoms of the substituent, the number of carbon atoms of the heterocyclic group is preferably 4 to 60, more preferably 4 to 30, and still more preferably 4 to 20.

As the divalent heterocyclic group, divalent aromatic heterocyclic groups are preferable. The divalent aromatic heterocyclic group is the remaining atomic group in which two hydrogen atoms are removed from an aromatic heterocyclic compound.

Examples of the divalent heterocyclic group include pyridinediyl groups such as a 2,5-pyridinediyl group and a 2,6-pyridinediyl group; quinolinediyl groups such as a 2,6-quinolinediyl group; isoquinolinediyl groups such as a 1,4-isoquinolinediyl group and a 1,5-isoquinolinediyl group; quinoxalinediyl groups such as a 5,8-quinoxalinediyl group; 2,1,3-benzothiadiazole groups such as a 2,1,3-benzothiadiazole-4,7-diyl group; benzothiazolediyl groups such as a 4,7-benzothiazolediyl group; dibenzosilolediyl groups such as a 2,7-dibenzosilolediyl group; dibenzofurandiyl groups such as a dibenzofuran-4,7-diyl group and a dibenzofuran-3,8-diyl group; and dibenzothiophenediyl groups such as a dibenzothiophene-4,7-diyl group and a dibenzothiophene-3,8-diyl group.

Examples of the “divalent group in which two or more same or different groups selected from arylene groups and divalent heterocyclic groups are linked” include divalent groups in which two groups selected from arylene groups and divalent heterocyclic groups are linked with a single bond such as a 2,7-biphenylylene group and a 3,6-biphenylylene group. The divalent group may have a substituent, and the substituent that the divalent group has is preferably a substituent selected from an alkyl group, an alkoxy group, an aryl group, an aryloxy group, a monovalent heterocyclic group, a heterocycleoxy group, and a halogen atom unless otherwise specified.

Hereinafter, suitable embodiments of the polymer compound, compound, composition, liquid composition, organic film, light-emitting device, surface light source, and display device according to the present invention will be described in detail.

(Polymer Compound)

The polymer compound according to the present embodiment has a first constitutional unit represented by the following formula (1) and a second constitutional unit represented by the following formula (2). The polymer compound is useful in production of the light-emitting device whose light emission efficiency is excellent because the polymer compound has these constitutional units.

It is preferable that the polymer compound according to the present embodiment be a conjugated polymer compound. The polymer compound according to the present embodiment may further have a third constitutional unit represented by the following formula (4). Such a polymer compound is more useful in production of the light-emitting device whose light emission efficiency is excellent. Here, the “conjugated polymer compound” is a polymer compound in which a conjugated system expands on the main chain skeleton, and examples thereof include polyarylenes having an arylene group such as polyfluorene and polyphenylene as a constitutional unit; polyheteroarylene having a divalent heterocyclic group such as polythiophene and polydibenzofuran as a constitutional unit; polyarylenevinylene such as polyphenylenevinylene; and copolymers having these constitutional units in combination. The “conjugated polymer compound” may be a compound substantially conjugated even if a hetero atom or the like is included in the main chain in the constitutional unit; for example, the “conjugated polymer compound” may include a constitutional unit derived from triarylamine as the constitutional unit.

Hereinafter, the first constitutional unit, the second constitutional unit, and the third constitutional unit each will be described in detail.

(First Constitutional Unit)

The first constitutional unit is the constitutional unit represented by the following formula (1):

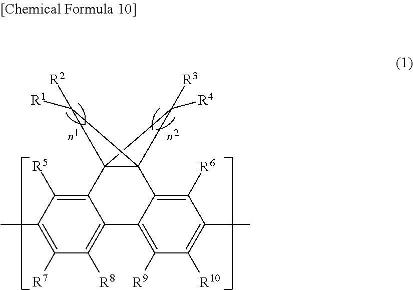

wherein n¹ and n² each independently represent an integer of 1 to 5; R¹, R², R³, R⁴, R⁵, R⁶, R⁷, R⁸, R⁹, and R¹⁰ each independently represent a hydrogen atom, an unsubstituted or substituted alkyl group, an unsubstituted or substituted alkoxy group, an unsubstituted or substituted aryl group, an unsubstituted or substituted aryloxy group, or an unsubstituted or substituted monovalent heterocyclic group.

As R¹, R², R³, and R⁴, the hydrogen atom, the unsubstituted or substituted alkyl group, and the unsubstituted or substituted aryl group are preferable, and the hydrogen atom and the unsubstituted or substituted alkyl group are more preferable because synthesis of the monomer is easy, and the light emission efficiency of the light-emitting device to be obtained is more excellent in the case where the polymer compound is used in production of the light-emitting device.

As R⁵, R⁶, R⁷, and R¹⁰, the hydrogen atom, the unsubstituted or substituted alkyl group, the unsubstituted or substituted aryl group are preferable, and it is more preferable that at least two thereof be a hydrogen atom because synthesis of the monomer is easy, and the light emission efficiency of the light-emitting device to be obtained is more excellent in the case where the polymer compound is used in production of the light-emitting device.

As R⁸ and R⁹, the hydrogen atom, the unsubstituted or substituted alkyl group, and the unsubstituted or substituted aryl group are preferable, and the hydrogen atom and the unsubstituted or substituted alkyl group are more preferable because the light emission efficiency of the light-emitting device to be obtained is more excellent in the case where the polymer compound is used in production of the light-emitting device.

In the formula (1), when n¹ is an integer of 2 to 5, a plurality of R¹ present may be the same or different from each other, and a plurality of R² present may be the same or different from each other. When n² is an integer of 2 to 5, a plurality of R³ present may be the same or different from each other, and a plurality of R⁴ present may be the same or different from each other.

Among R¹, R², R³, and R⁴, adjacent groups may be linked to each other to form a cyclic structure. Among R⁷, R⁸, R⁹, and R¹⁰, adjacent groups may be linked to each other to form a cyclic structure.

It is preferable that the content of the first constitutional unit be 0.5 mol % or more of the total constitutional units, it is more preferable that the content of the first constitutional unit be 0.5 to 80 mol % of the total constitutional units, and it is still more preferable that the content of the first constitutional unit be 5 to 60 mol % of the total constitutional units because the light emission efficiency of the light-emitting device to be obtained is more excellent in the case where the polymer compound is used in production of the light-emitting device.

In the first constitutional unit, stereoisomerism can be produced when n¹ and/or n² is 2 or more and the first constitutional unit has a substituent, when R¹ and R² are different from each other, and when R³ and R⁴ are different from each other. As the first constitutional unit, the polymer compound may have only a constitutional unit having the same stereoisomerism, or may have a plurality of constitutional units having stereoisomerism different from each other. Examples of the stereoisomerism include diastereoisomers and enantiomers.

In the case where the first constitutional unit is represented by the formula (1-A), examples of the stereoisomerism are represented by the following formula (1-a), the formula (1-b), the formula (1-c), and the formula (1-d). In the following formulas, R^{a} and R^{b} each independently represent an alkyl group.

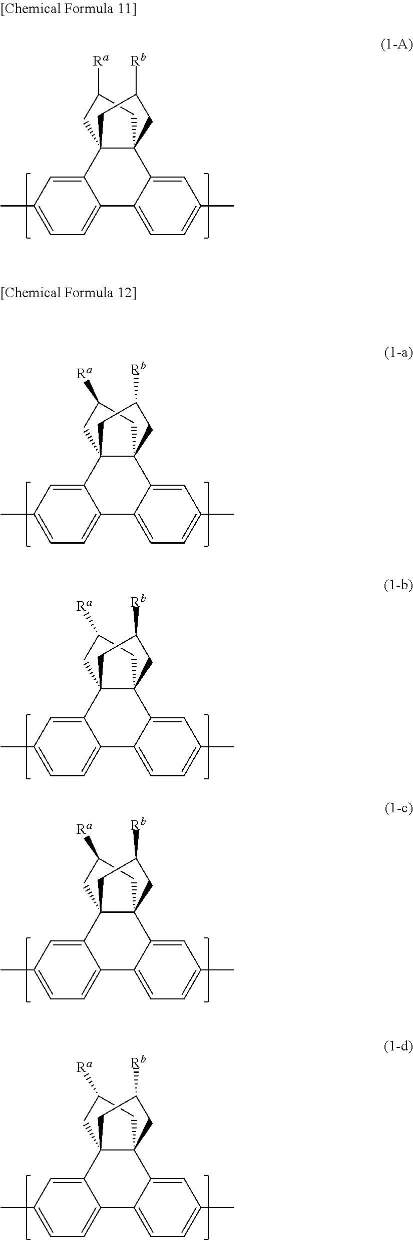

The constitutional unit represented by the formula (1-a), the constitutional unit represented by the formula (1-b), the constitutional unit represented by the formula (1-c) and the constitutional unit represented by the formula (1-d) are in the relationship of diastereoisomers.

In the formula (1), in the case where the group represented by R², R³, R⁴, R⁵, R⁶, R⁷, R⁸, R⁹, and R¹⁰ has a substituent, the substituent is preferably an alkyl group, an alkoxy group, an aryl group, an aryloxy group, an arylalkyl group, an arylalkoxy group, an arylalkenyl group, an arylalkynyl group, an amino group, a substituted amino group, a halogen atom, an acyl group, an acyloxy group, a monovalent heterocyclic group, a carboxyl group, a nitro group, and a cyano group, more preferably an alkyl group, an alkoxy group, an aryl group, an aryloxy group, an arylalkyl group, an arylalkoxy group, a substituted amino group, an acyl group, and a cyano group, and still more preferably an alkyl group, an alkoxy group, and an aryl group.

In the formula (1), R¹, R², R³, and R⁴ can be a hydrogen atom, an unsubstituted or substituted alkyl group, or an unsubstituted or substituted aryl group. Here, as the substituted alkyl group in R¹, R², R³, and R⁴, an arylalkyl group or an alkylarylalkyl group can be selected; as the substituted aryl group in R¹, R², R³, and R⁴, an alkylaryl group can be selected.

In the formula (1), R⁵, R⁶, R⁷, R⁸, R⁹, and R¹⁰ can be a hydrogen atom, an unsubstituted or substituted alkyl group, or an unsubstituted or substituted aryl group. Here, as the substituted alkyl group in R⁵, R⁶, R⁷, R⁸, R⁹, and R¹⁰, an arylalkyl group or an alkylarylalkyl group can be selected; as the substituted aryl group in R⁵, R⁶, R⁷, R⁸, R⁹, and R¹⁰, an alkylaryl group can be selected.

In the formula (1), “among R¹, R², R³, and R⁴, adjacent groups may be linked to each other to form a cyclic structure” means that among R¹, R², R³, and R⁴, groups bonded to the same carbon atom may be linked to each other to form a cyclic structure, or when n¹ and/or n² is 2 or more, groups bonded to carbon atoms in adjacent positions may be linked to each other to form a cyclic structure.

In the formula (1), “among R⁷, R⁸, R⁹, and R¹⁰, adjacent groups may be linked to each other to form a cyclic structure” means that groups bonded to carbon atoms in adjacent positions may be linked to each other to form a cyclic structure, and for example, R⁸ and R⁹ may be linked to form a cyclic structure. Namely, the first constitutional unit can have a structure represented by, for example, the following formula (1-d), (1-e), (1-f), (1-g), (1-h), or (1-i):

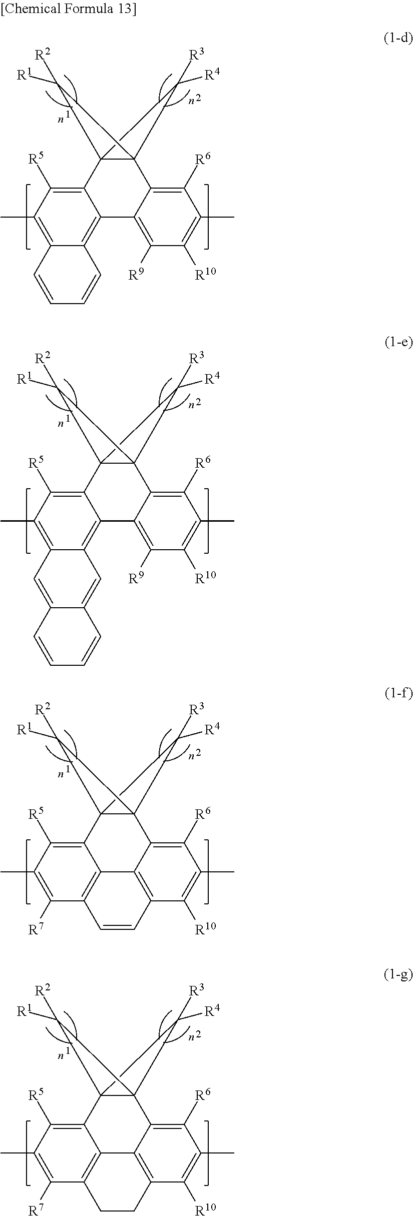

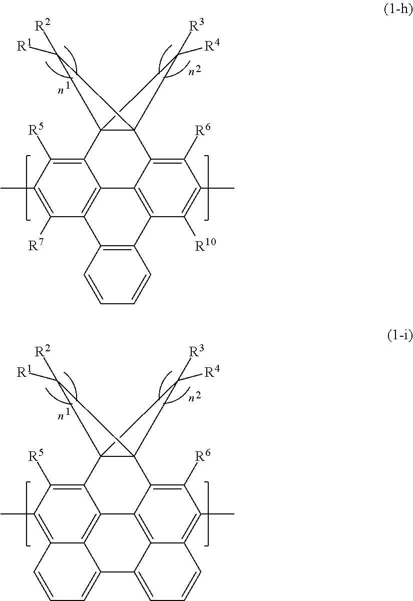

The structure represented by the formula (1-d) and the structure represented by the formula (1-e) are examples in which R⁷ and R⁸ in the formula (1) are linked to each other to form a cyclic structure; the structure represented by the formula (1-f), the structure represented by (1-g), and the structure represented by the formula (1-h) are examples in which R⁸ and R⁹ in the formula (1) are linked to each other to form a cyclic structure; the structure represented by the formula (1-i) is an example in which R⁷, R⁸, R⁹, and R¹⁰ are linked to each other to form a cyclic structure.

The formed cyclic structure may have a substituent; the substituent is preferably an alkyl group, an alkoxy group, an aryl group, an aryloxy group, an arylalkyl group, an arylalkoxy group, an arylalkenyl group, an arylalkynyl group, an amino group, a substituted amino group, a halogen atom, an acyl group, an acyloxy group, a monovalent heterocyclic group, a carboxyl group, a nitro group, and a cyano group, more preferably an alkyl group, an alkoxy group, an aryl group, an aryloxy group, an arylalkyl group, an arylalkoxy group, a substituted amino group, and an acyl group, cyano group, and still more preferably an alkyl group, an alkoxy group, and an aryl group.

In the formula (1), because the light emission efficiency of the light-emitting device using the polymer compound according to the present embodiment is more excellent, it is preferable that n¹ and n² be an integer of 3 to 5, it is more preferable that n¹ and n² be an integer of 3 or 4, and it is still more preferable that n¹ and n² be 3. n¹ and n² may be the same or different from each other; it is preferable that n¹ and n² be the same because production of the monomer is easy.

Examples of the constitutional unit represented by the formula (1) include the constitutional unit represented by the following formula (1A):

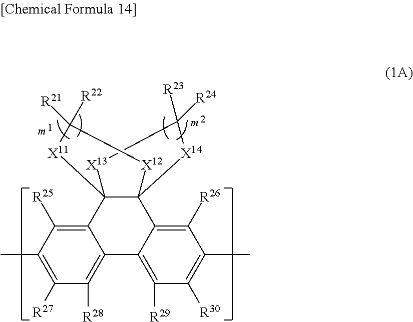

In the formula (1A), m¹ and m² each independently represent 1 or 2. R²¹, R²², R²³, R²⁴, R²⁵, R²⁶, R²⁷, R²⁸, R²⁹, and R³⁰ are the same as R¹ to R¹⁰. When R²¹, R²², R²³, and R²⁴ exist in plural, the plurality of R²¹, R²², R²³, or R²⁴ may be the same or different from each other. Among R²¹, R²², R²³, and R²⁴, adjacent groups may be linked to each other to form a cyclic structure. Among R²⁷, R²⁸, R²⁹, and R³⁰, adjacent groups may be linked to each other to form a cyclic structure. X¹¹, X¹², X¹³, and X¹⁴ each independently represent a group represented by —C(R³¹)₂—. Here, R³¹ is the same as R¹ to R⁴, and a plurality of R³¹ present may be the same or different from each other.

It is preferable that m¹ and m² be the same because synthesis of the monomer is easy, and it is more preferable that m¹ and m² be 1 because synthesis of the monomer is easy, and the light emission efficiency of the light-emitting device to be obtained is more excellent in the case where the polymer compound is used in production of the light-emitting device.

As R²¹, R²², R²³, and R²⁴, a hydrogen atom, an unsubstituted or substituted alkyl group, and an unsubstituted or substituted aryl group are preferable because the light emission efficiency of the light-emitting device to be obtained is more excellent in the case where the polymer compound is used in production of the light-emitting device; it is more preferable that at least one be a group other than a hydrogen atom because the solubility of the polymer compound in a solvent is improved and production of the device is easier, and the light emission efficiency of the light-emitting device to be obtained is more excellent in the case where the polymer compound is used in production of the light-emitting device.

As R³¹, a hydrogen atom and a substituted or unsubstituted alkyl group are preferable because synthesis of the monomer is easy, and the light emission efficiency of the light-emitting device to be obtained is more excellent in the case where the polymer compound is used in production of the light-emitting device. Among a plurality of R³¹ present, it is preferable that at least one be a hydrogen atom, and it is more preferable that all be the hydrogen atom.

As R²⁵, R²⁶, R²⁷, R²⁸, R²⁹, and R³⁰, a hydrogen atom, an unsubstituted or substituted alkyl group, and an unsubstituted or substituted aryl group are preferable, and it is more preferable that at least two be a hydrogen atom because synthesis of the monomer is easy, and the light emission efficiency of the light-emitting device to be obtained is more excellent in the case where the polymer compound is used in production of the light-emitting device.

Among the constitutional units represented by the formula (IA), the constitutional unit in which among R²¹, R²², R²³ and R²⁴, at least one is a hydrogen atom can be easily derived from a compound represented by the formula (6) described later.

Examples of the first constitutional unit include constitutional units represented by the following formulas (1-1) to (1-28). Among the constitutional units represented by the formulas (1-1) to (1-28), the constitutional units represented by the formulas (1-2), (1-3), (1-4), (1-6), (1-7), (1-8), (1-9), (1-10), (1-11), (1-12), (1-13), (1-14), (1-15), (1-16), (1-18), (1-19), (1-20), (1-22), (1-23), (1-25), (1-26), and (1-27) are preferable, the constitutional units represented by the formulas (1-2), (1-3), (1-4), (1-6), (1-7), (1-8), (1-9), (1-10), (1-11), (1-12), (1-13), (1-14), (1-15), (1-18), (1-19), (1-20), (1-23), (1-25), (1-26), and (1-27) are more preferable, and the constitutional units represented by the formulas (1-4), (1-8), (1-9), (1-10), (1-12), (1-14), (1-15), (1-25), and (1-26) are still more preferable because synthesis of the monomer is easy, and the light emission efficiency of the light-emitting device to be obtained is more excellent in the case where the polymer compound is used in production of the light-emitting device.

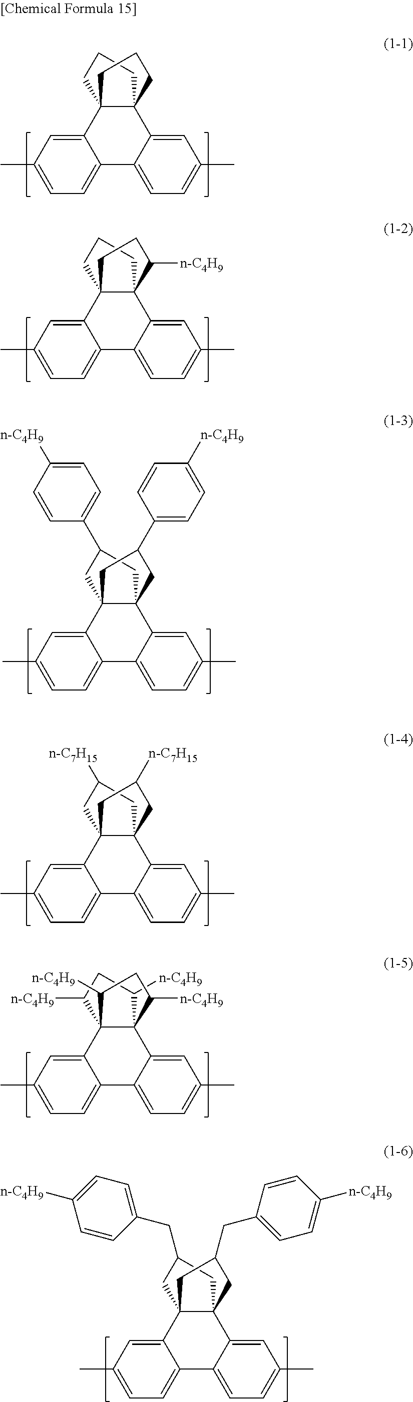

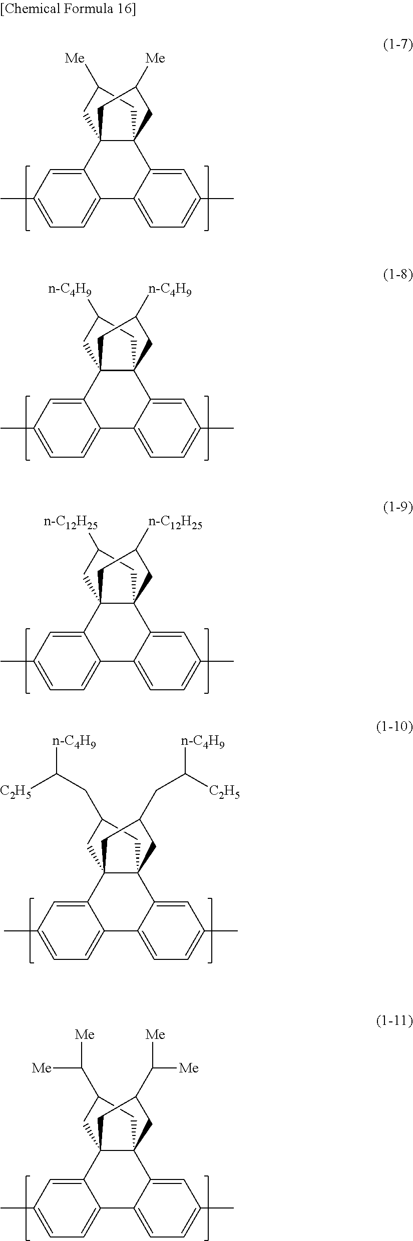

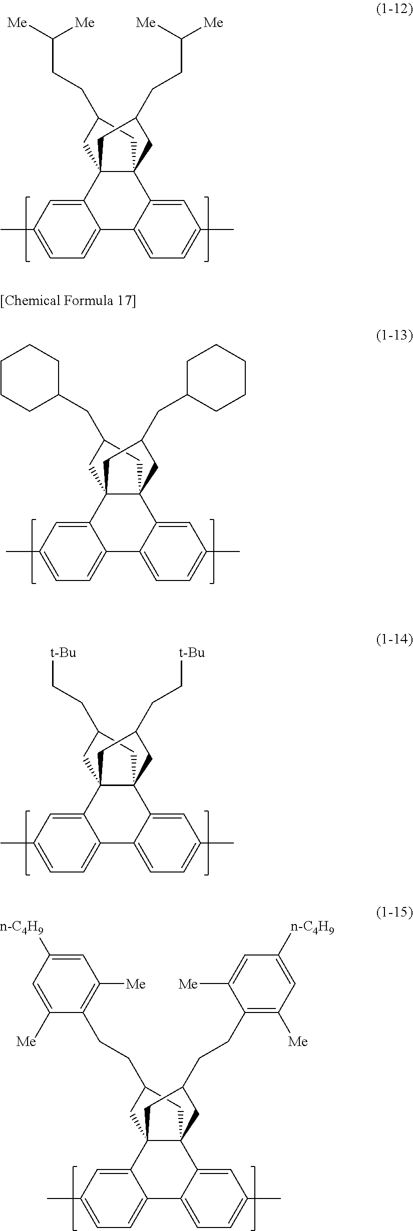

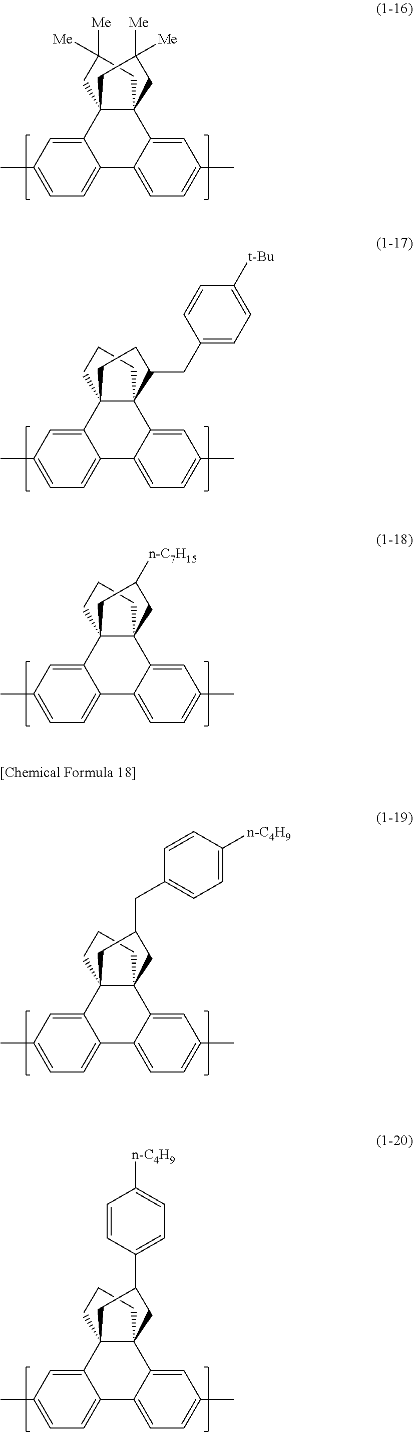

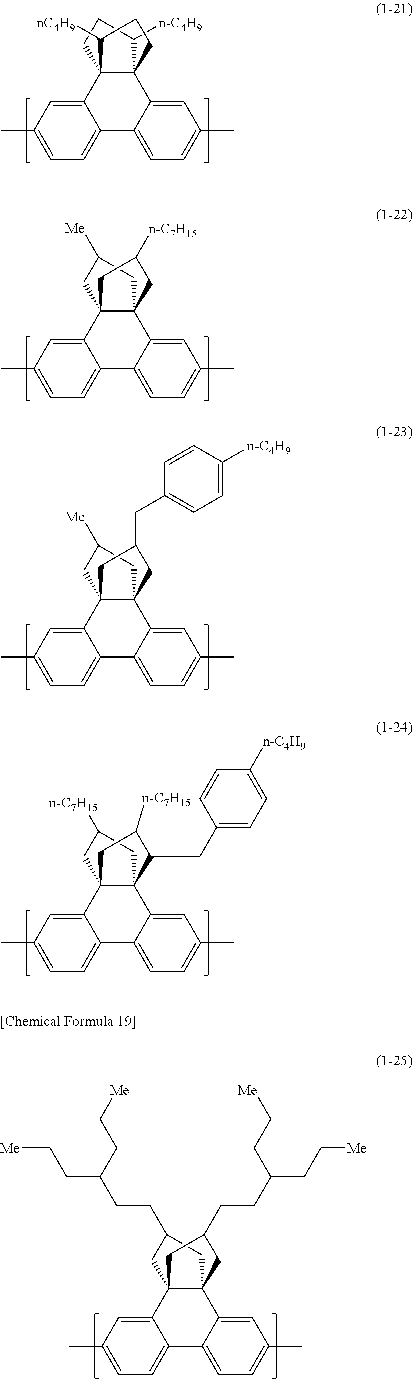

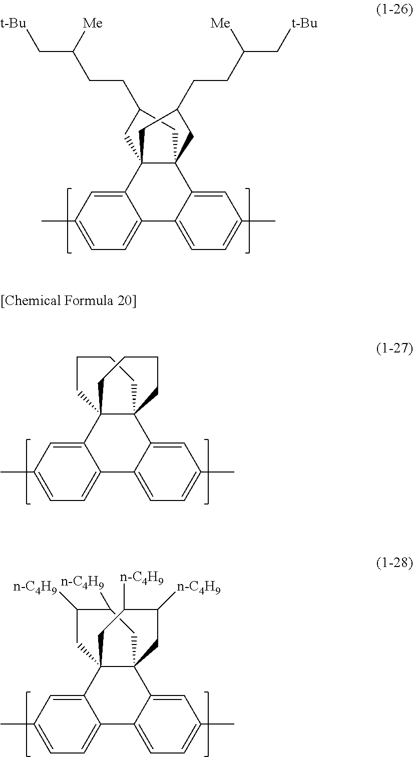

As the first constitutional unit, the polymer compound may have only one constitutional unit above, or may have a plurality of different constitutional units among the constitutional units above.

(Second Constitutional Unit)

The second constitutional unit is a constitutional unit represented by the following formula (2):

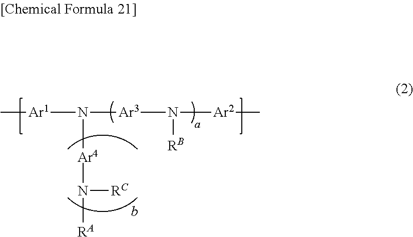

In the formula (2), a and b each independently represent 0 or 1. Ar¹, Ar², Ar³, and Ar⁴ each independently represent an “unsubstituted or substituted arylene group,” an “unsubstituted or substituted divalent heterocyclic group,” or a “divalent group in which two or more same or different groups selected from arylene groups and divalent heterocyclic groups are linked (the group may have a substituent).” R^{A}, R^{B}, and R^{C} each independently represent a hydrogen atom, an unsubstituted or substituted alkyl group, an unsubstituted or substituted aryl group, or an unsubstituted or substituted monovalent heterocyclic group. Ar¹, Ar², Ar³, and Ar⁴ each may be bonded to a group other than the group, which is bonded to a nitrogen atom to which the group is bonded, to form a cyclic structure.

In the formula (2), it is preferable that a be 1 because the light emission efficiency of the light-emitting device using the polymer compound according to the present embodiment is more excellent.

In the formula (2), it is preferable that b be 0 because synthesis of the monomer is easy, and the light emission efficiency of the light-emitting device using the polymer compound according to the present embodiment is more excellent.

In the formula (2), it is preferable that R^{A}, R^{B}, and R^{C} be a substituted alkyl group, an unsubstituted or substituted aryl group, or an unsubstituted or substituted monovalent heterocyclic group, and it is more preferable that R^{A}, R^{B}, and R^{C} be an unsubstituted or substituted aryl group because the stability of the polymer compound according to the present embodiment is good, and the light emission efficiency of the light-emitting device using the polymer compound is more excellent.

In the formula (2), in the case where a group represented by Ar¹, Ar², Ar³, and Ar⁴ has a substituent, examples of the substituent include an alkyl group, an alkoxy group, an aryl group, an aryloxy group, an arylalkyl group, an arylalkoxy group, an arylalkenyl group, an arylalkynyl group, an amino group, a substituted amino group, a halogen atom, an acyl group, an acyloxy group, a monovalent heterocyclic group, a carboxyl group, a nitro group, and a cyano group; it is preferable that the substituent be an alkyl group, an alkoxy group, an aryl group, an aryloxy group, an arylalkyl group, an arylalkoxy group, a substituted amino group, an acyl group, and a cyano group, and it is more preferable that the substituent be an alkyl group, an alkoxy group, and an aryl group.

In the formula (2), it is preferable that the group represented by Ar¹, Ar², Ar³, and Ar⁴ be an unsubstituted or substituted arylene group, or an unsubstituted or substituted divalent heterocyclic group, and particularly an unsubstituted or substituted arylene group because the stability of the polymer compound according to the present embodiment is good, and the light emission efficiency of the light-emitting device using the polymer compound is more excellent.

In the formula (2), examples of the arylene group in Ar¹, Ar², Ar³, and Ar⁴ include a 1,2-phenylene group, a 1,3-phenylene group, a 1,4-phenylene group, a 1,4-naphthalenediyl group, a 2,6-naphthalenediyl group, a 2,7-naphthalenediyl group, a 2,6-anthracenediyl group, a 9,10-anthracenediyl group, a 2,7-phenanthrenediyl group, a 5,12-naphthacenediyl group, 2,7-fluorenediyl, a 3,6-fluorenediyl group, a 1,6-pyrenediyl group, a 2,7-pyrenediyl group, and a 3,8-perylenediyl group; the 1,4-phenylene group, 2,7-fluorenediyl, the 2,6-anthracenediyl group, the 9,10-anthracenediyl group, the 2,7-phenanthrenediyl group, and the 1,6-pyrenediyl group are preferable, and may have the substituent above.

In the formula (2), examples of the divalent heterocyclic group in Ar¹, Ar², Ar³, and Ar⁴ include a 2,5-pyrrolediyl group, a dibenzofurandiyl group, a dibenzothiophenediyl group, and a 2,1,3-benzothiadiazole-4,7-diyl group, and may have the substituent above. In the divalent heterocyclic group in Ar¹, Ar², Ar³, and Ar⁴, the group represented by the formula (3) described later is not included.

In the formula (2), as the divalent group in which two or more same or different groups selected from arylene groups and divalent heterocyclic groups are linked in Ar¹, Ar², Ar³, and Ar⁴, a group represented by the following formula (2a-1), (2a-2), (2a-3), (2a-4), (2a-5), (2a-6), or (2a-7) can be selected; the group represented by the following formula (2a-1) is preferable, and may have the substituent above.

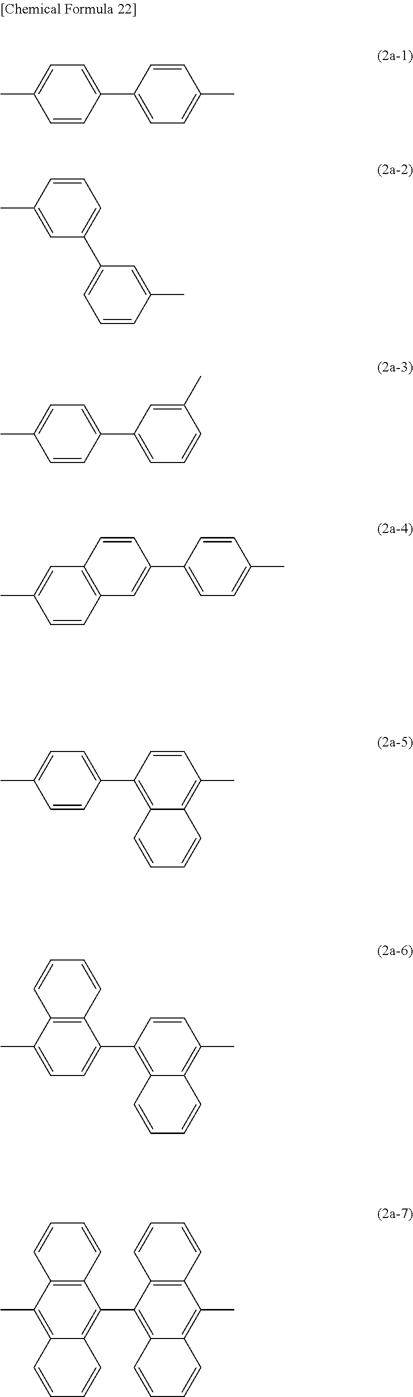

In the formula (2), in the case where the group represented by R^{A}, R^{B}, and R^{C} has a substituent, the substituent is preferably an alkyl group, an alkoxy group, an aryl group, an aryloxy group, an arylalkyl group, an arylalkoxy group, an arylalkenyl group, an arylalkynyl group, an amino group, a substituted amino group, a halogen atom, an acyl group, an acyloxy group, a monovalent heterocyclic group, a carboxyl group, a nitro group, a cyano group, and more preferably an alkyl group, an alkoxy group, an aryl group, an aryloxy group, an arylalkyl group, an arylalkoxy group, a substituted amino group, an acyl group, and a cyano group, and still more preferably an alkyl group, an alkoxy group, and an aryl group.

In the formula (2), examples of the alkyl group in R^{A}, R^{B}, and R^{C} include C₁ to C₂₀ alkyl groups. The alkyl group may have the substituent above.

In the formula (2), examples of the aryl group in R^{A}, R^{B}, and R^{C} include a phenyl group, a 1-naphthyl group, a 2-naphthyl group, a 1-anthracenyl group, a 2-anthracenyl group, a 9-anthracenyl group, and a 2-fluorenyl group, and may have the substituent above.

In the formula (2), examples of the monovalent heterocyclic group in R^{A}, R^{B}, and R^{C} include a pyridyl group, a pyrimidyl group, a triazyl group, and a quinolyl group, and may have the substituent above.

It is preferable that the content of the second constitutional unit be 0.1 mol % or more of the total constitutional units, it is more preferable that the content of the second constitutional unit be 0.1 to 50 mol % of the total constitutional units, and it is still more preferable that the content of the second constitutional unit be 0.1 to 40 mol % of the total constitutional units because the light emission efficiency of the light-emitting device to be obtained is more excellent in the case where the polymer compound is used in production of the light-emitting device.

Examples of the second constitutional unit include constitutional units represented by the following formulas (2-a), (2-b), (2-c), and (2-d); the constitutional units represented by the formulas (2-b), (2-c), and (2-d) are preferable, and the constitutional unit represented by the formula (2-c) is more preferable because the light emission efficiency of the light-emitting device using the polymer compound according to the present embodiment is more excellent.

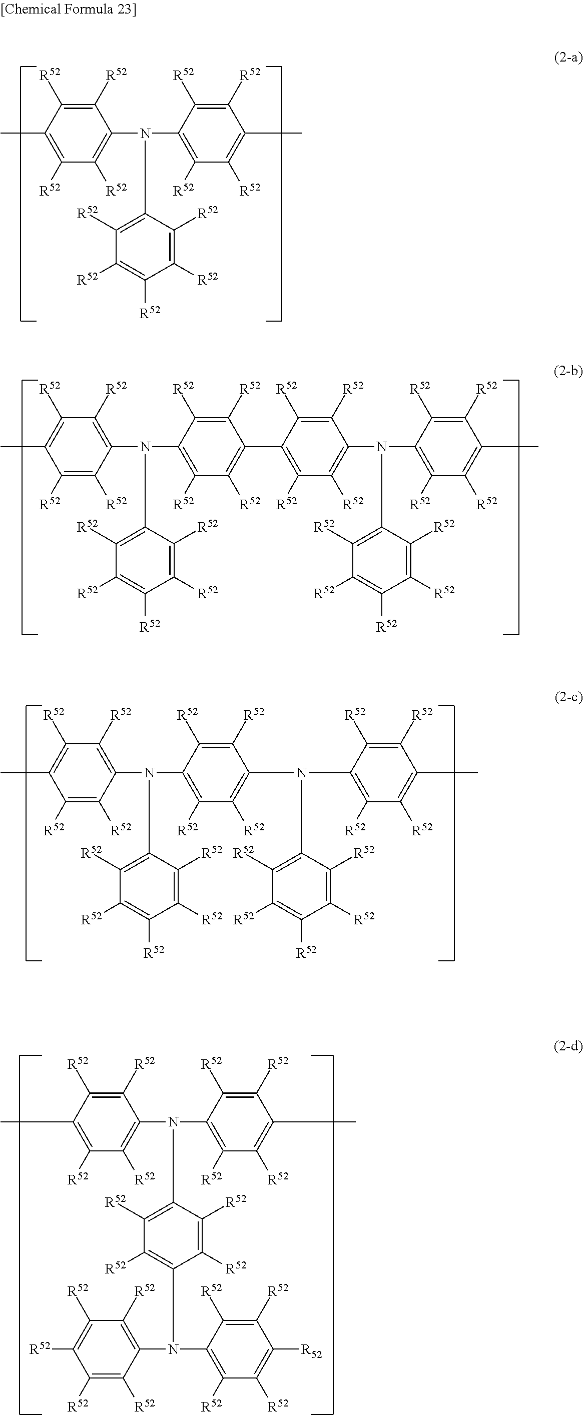

In the formulas (2-a) to (2-d), R⁵² represents a hydrogen atom, an alkyl group, an alkoxy group, an aryl group, an aryloxy group, an arylalkyl group, an arylalkoxy group, an arylalkenyl group, an arylalkynyl group, an amino group, a substituted amino group, a halogen atom, an acyl group, an acyloxy group, a monovalent heterocyclic group, a carboxyl group, a nitro group, or a cyano group. R⁵² is preferably an alkyl group, an alkoxy group, an aryl group, an aryloxy group, an arylalkyl group, an arylalkoxy group, a substituted amino group, and an acyl group, a cyano group, and more preferably an alkyl group, an alkoxy group, and an aryl group. A plurality of R⁵² present may be the same or different from each other. Among the plurality of R⁵² present, adjacent groups may be linked to each other to form a cyclic structure.

As the second constitutional unit, the constitutional unit represented by the following formula (2A) is also preferable.

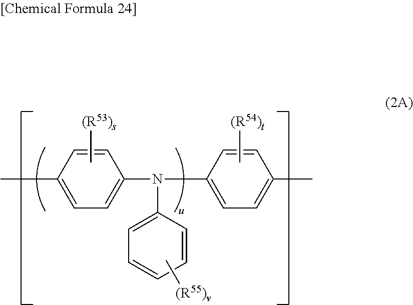

wherein s and t each independently represent an integer of 0 to 4; u is 1 or 2; v is an integer of 0 to 5; R⁵³, R⁵⁴, and R⁵⁵ each independently represent alkyl group, an alkoxy group, an aryl group, an aryloxy group, an arylalkyl group, an arylalkoxy group, an arylalkenyl group, an arylalkynyl group, an amino group, a substituted amino group, a halogen atom, an acyl group, an acyloxy group, a monovalent heterocyclic group, a carboxyl group, a nitro group, or a cyano group. When R⁵³, R⁵⁴, and R⁵⁵ exist in plural, the plurality of groups present may be the same or different from each other. Among the plurality of R⁵³ present, adjacent groups may be linked to each other to form a cyclic structure. Among the plurality of R⁵⁴ present, adjacent groups may be linked to each other to form a cyclic structure.

In the formula (2A), it is preferable that s and t be 0 to 2, u be 2, and v be 1 to 5 because the light emission efficiency of the light-emitting device using the polymer compound according to the present embodiment is more excellent. v is more preferably 1 to 3.

In the formula (2A), it is preferable that R⁵³, R⁵⁴ and R⁵⁵ be an alkyl group, an alkoxy group, or an aryl group because the light emission efficiency of the light-emitting device using the polymer compound according to the present embodiment is more excellent.

The second constitutional unit may be the constitutional unit represented by the following formula (3).

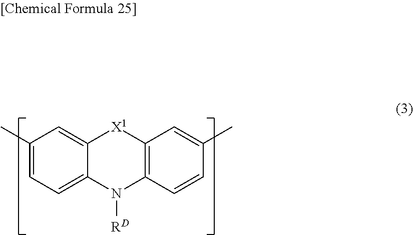

wherein R^{D} represents a hydrogen atom, an unsubstituted or substituted alkyl group, an unsubstituted or substituted aryl group, or an unsubstituted or substituted monovalent heterocyclic group; X¹ represents a single bond, an oxygen atom, a sulfur atom, or a group represented by —C(R¹¹)₂—; R¹¹ represents an unsubstituted or substituted alkyl group or an unsubstituted or substituted aryl group; and a plurality of R¹¹ present may be the same or different from each other.

It is preferable that R^{D} be an unsubstituted or substituted alkyl group, an unsubstituted or substituted aryl group, or an unsubstituted or substituted monovalent heterocyclic group, it is more preferable that R^{D} be an unsubstituted or substituted alkyl group, or an unsubstituted or substituted aryl group, and it is still more preferable that R^{D} be an unsubstituted or substituted aryl group because the stability of the polymer compound according to the present embodiment is good, and the light emission efficiency of the light-emitting device using the polymer compound is more excellent.

It is preferable that X¹ be a single bond or an oxygen atom, and it is more preferable that X¹ be an oxygen atom because the light emission efficiency of the light-emitting device to be obtained is more excellent in the case where the polymer compound is used in production of the light-emitting device.

In the case where the group represented by R^{D} in the formula (3) has a substituent, the substituent is preferably an alkyl group, an alkoxy group, an aryl group, an aryloxy group, an arylalkyl group, an arylalkoxy group, an arylalkenyl group, an arylalkynyl group, an amino group, a substituted amino group, a halogen atom, an acyl group, an acyloxy group, monovalent heterocyclic group, a carboxyl group, a nitro group, and a cyano group, more preferably an alkyl group, an alkoxy group, an aryl group, an aryloxy group, an arylalkyl group, an arylalkoxy group, a substituted amino group, an acyl group, a cyano group, and still more preferably an alkyl group, an alkoxy group, and an aryl group.

In the formula (3), examples of the alkyl group in R^{D} include C₁ to C₂₀ alkyl groups, and the alkyl group may have the substituent above.

In the formula (3), examples of the aryl group in R^{D} include a phenyl group, a 1-naphthyl group, a 2-naphthyl group, a 1-anthracenyl group, a 2-anthracenyl group, a 9-anthracenyl group, and a 2-fluorenyl group, and the aryl group may have the substituent above.

In the formula (3), examples of the heterocyclic group in R^{D} include a pyridyl group, a pyrimidyl group, a triazyl group, and a quinolyl group, and the heterocyclic group may have the substituent above.

In the case where the group represented by R¹¹ in the formula (3) has a substituent, the substituent is preferably an alkyl group, an alkoxy group, an aryl group, an aryloxy group, an arylalkyl group, an arylalkoxy group, an arylalkenyl group, an arylalkynyl group, an amino group, a substituted amino group, a halogen atom, an acyl group, an acyloxy group, a heterocyclic group, a carboxyl group, a nitro group, and a cyano group, more preferably an alkyl group, an alkoxy group, an aryl group, an aryloxy group, an arylalkyl group, an arylalkoxy group, a substituted amino group, an acyl group, a cyano group, and still more preferably an alkyl group, an alkoxy group, and an aryl group.

In the formula (3), examples of the alkyl group in R¹¹ include C₁ to C₂₀ alkyl groups, and the alkyl group may have the substituent above.

In the formula (3), examples of the aryl group in R^{H} include a phenyl group, a 1-naphthyl group, a 2-naphthyl group, a 1-anthracenyl group, a 2-anthracenyl group, a 9-anthracenyl group, and a 2-fluorenyl group, and the aryl group may have the substituent above.

Examples of the second constitutional unit include constitutional units represented by the following formulas (2-1) to (2-12). Among the constitutional units represented by the formulas (2-1) to (2-12), the constitutional unit represented by the formulas (2-1), (2-2), (2-3), (2-4), (2-5), (2-6), (2-7), (2-8), (2-9), (2-10), and (2-12) are preferable, the constitutional units represented by the formulas (2-1), (2-2), (2-4), (2-5), (2-6), (2-7), (2-8), (2-9), and (2-10) are more preferable, and the constitutional units represented by the formulas (2-2), (2-4), (2-8), and (2-9) are still more preferable because the light emission efficiency of the light-emitting device to be obtained is more excellent in the case where the polymer compound is used in production of the light-emitting device.

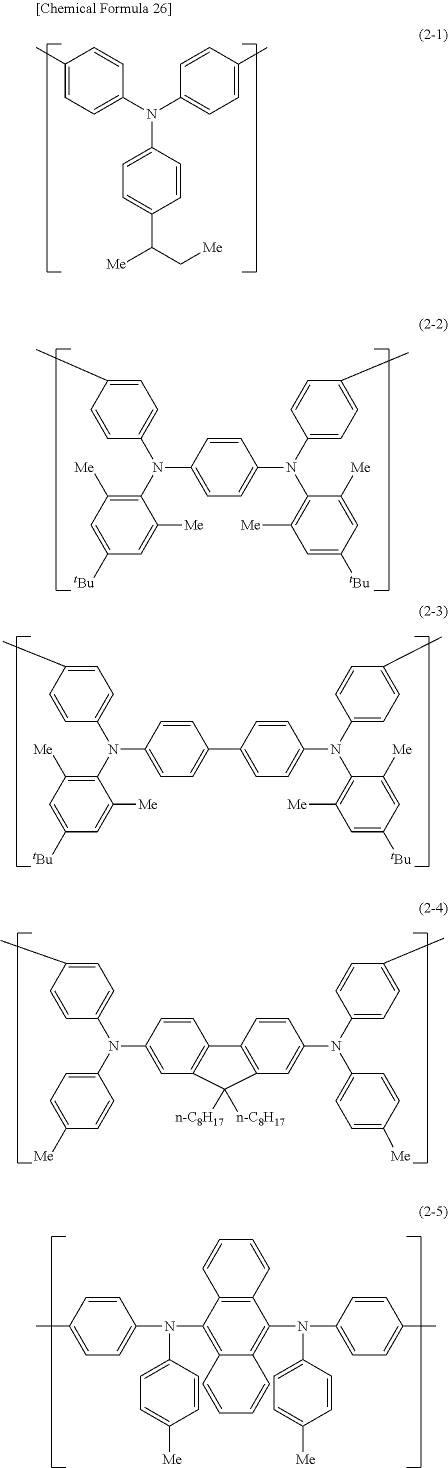

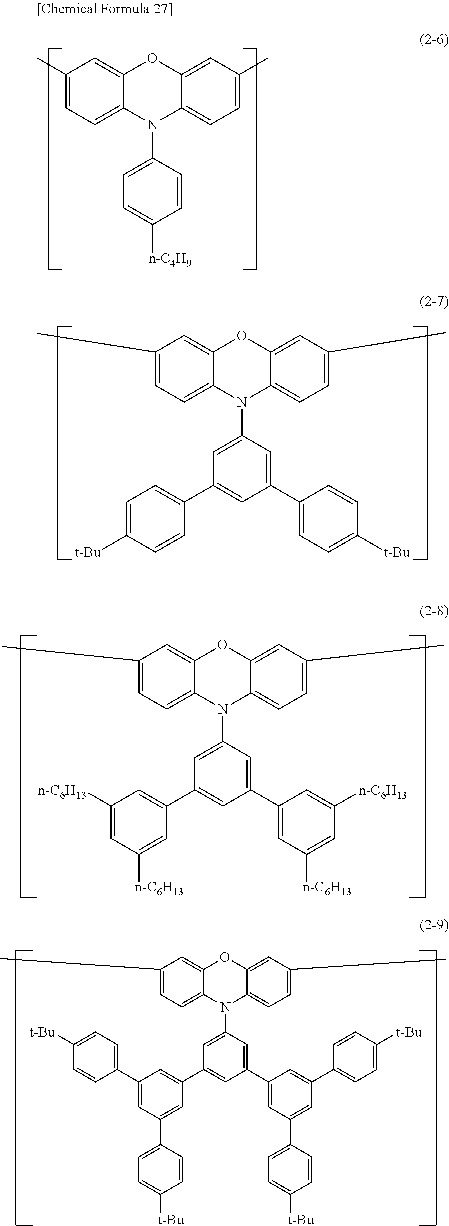

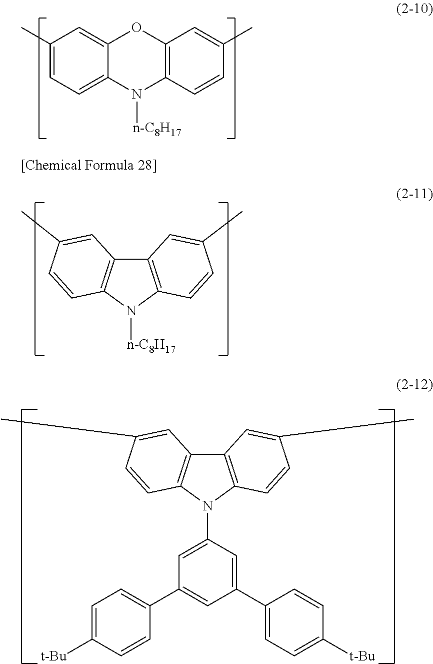

As the second constitutional unit, the polymer compound may have only one constitutional unit above, or may have a plurality of different constitutional units among the constitutional units above.

(Third Constitutional Unit)

The third constitutional unit is a constitutional unit represented by the following formula (4):

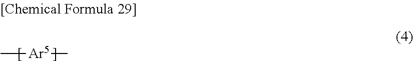

In the formula (4), Ar⁵ represents an unsubstituted or substituted arylene group, an unsubstituted or substituted divalent heterocyclic group, or a divalent group in which two or more same or different groups selected from arylene groups and divalent heterocyclic groups are linked (the group may have a substituent), and it is preferable that Ar⁵ be an unsubstituted or substituted arylene group or an unsubstituted or substituted divalent heterocyclic group. The constitutional unit represented by the formula (4) is different from the constitutional unit represented by the above formula (3).

In the case where the group represented by Ar⁵ in the formula (4) has a substituent, the substituent is preferably an alkyl group, an alkoxy group, an aryl group, an aryloxy group, an arylalkyl group, an arylalkoxy group, an arylalkenyl group, an arylalkynyl group, an amino group, a substituted amino group, a halogen atom, an acyl group, an acyloxy group, a monovalent heterocyclic group, a carboxyl group, a nitro group, and a cyano group, more preferably an alkyl group, an alkoxy group, an aryl group, an aryloxy group, an arylalkyl group, an arylalkoxy group, a substituted amino group, an acyl group, a cyano group, and still more preferably an alkyl group, an alkoxy group, and an aryl group.

In the formula (4), examples of the arylene group in Ar⁵ include a 1,2-phenylene group, a 1,3-phenylene group, a 1,4-phenylene group, a 1,4-naphthalenediyl group, a 2,6-naphthalenediyl group, a 2,7-naphthalenediyl group, a 2,6-anthracenediyl group, a 9,10-anthracenediyl group, a 2,7-phenanthrenediyl group, 5,12-naphthacenediyl group, a 2,7-fluorenediyl group, a 3,6-fluorenediyl group, and a 3,8-perylenediyl group, and the arylene group may have the substituent above.

As Ar⁵ in the formula (4), a 1,3-phenylene group, a 1,4-phenylene group, a 2,6-naphthalenediyl group, a 2,7-naphthalenediyl group, a 2,6-anthracenediyl group, a 9,10-anthracenediyl group, a 2,7-phenanthrenediyl group, a 2,7-fluorenediyl group, and a 3,6-fluorenediyl group are preferable because the light emission efficiency of the light-emitting device to be obtained is more excellent in the case where the polymer compound according to the present embodiment is used in production of the light-emitting device.

In the formula (4), examples of the divalent heterocyclic group in Ar⁵ include a 2,5-pyrrolediyl group, a 2,1,3-benzothiadiazole-4,7-diyl group, a dibenzofurandiyl group, and a dibenzothiophenediyl group, and the divalent heterocyclic group may have the substituent above.

In the formula (4), examples of the divalent group in which two or more same or different groups selected from arylene groups and divalent heterocyclic groups are linked in Ar^{y} include a group represented by the above formula (2a-1), (2a-2), (2a-3), (2a-4), (2a-5), (2a-6), or (2a-7), and the divalent group may have the substituent above.

Examples of the third constitutional unit include the constitutional units represented by the following formulas (3-1) to (3-35). Among the constitutional units represented by the formulas (3-1) to (3-36), the constitutional units represented by the formulas (3-1), (3-2), (3-3), (3-4), (3-5), (3-6), (3-7), (3-8), (3-9), (3-10), (3-11), (3-12), (3-13), (3-14), (3-21), (3-22), (3-23), (3-25), (3-27), (3-28), (3-30), (3-32), (3-33), (3-35), and (3-36) are preferable, the constitutional units represented by the formulas (3-1), (3-2), (3-3), (3-4), (3-5), (3-6), (3-7), (3-8), (3-9), (3-10), (3-11), (3-12), (3-13), (3-14), (3-28), and (3-30) are more preferable, and the constitutional units represented by the formulas (3-1), (3-2), (3-4), (3-5), (3-12), (3-13), (3-14), and (3-30) are still more preferable because the light emission efficiency of the light-emitting device to be obtained is more excellent in the case where the polymer compound is used in production of the light-emitting device.

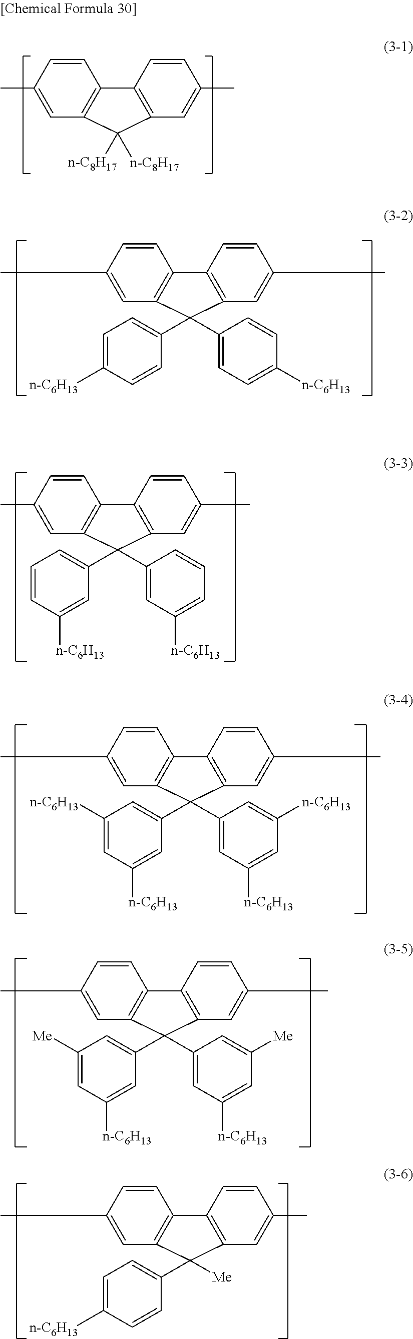

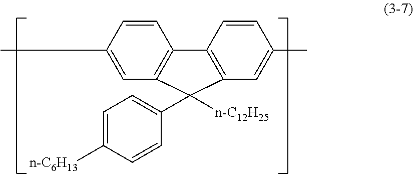

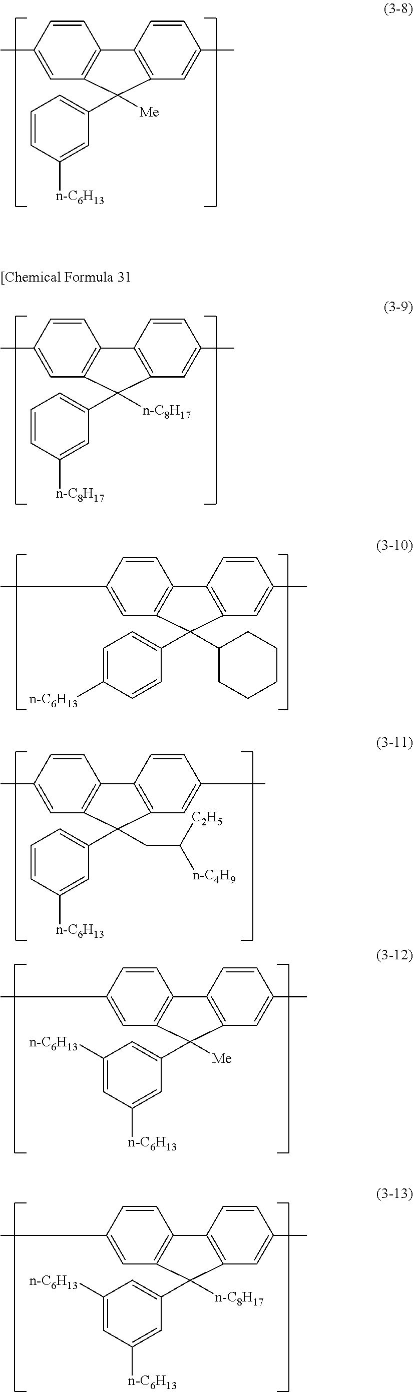

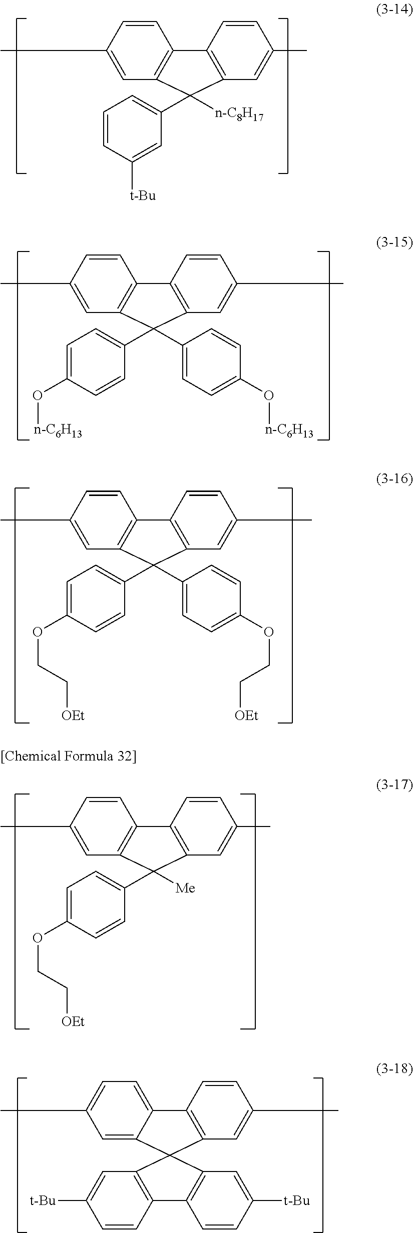

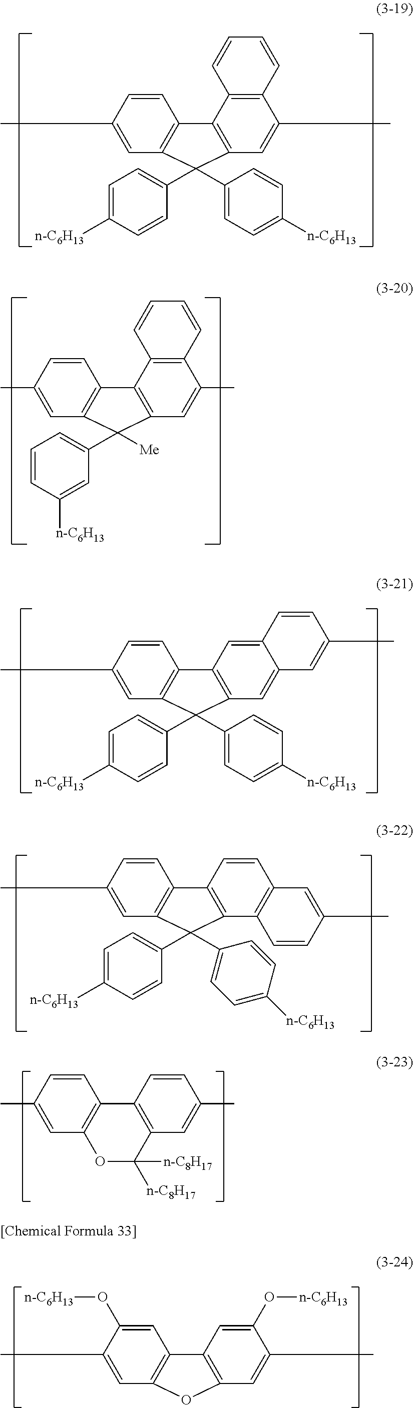

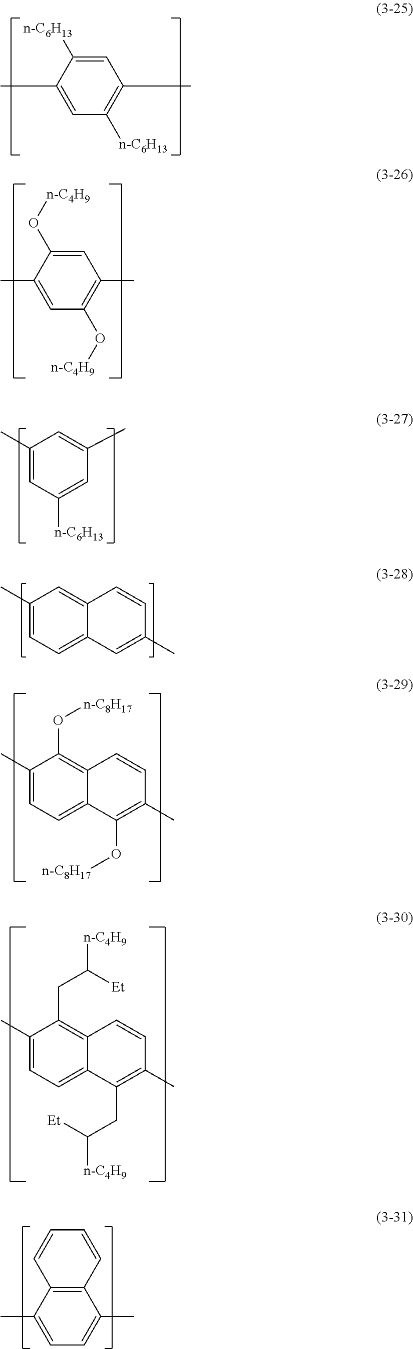

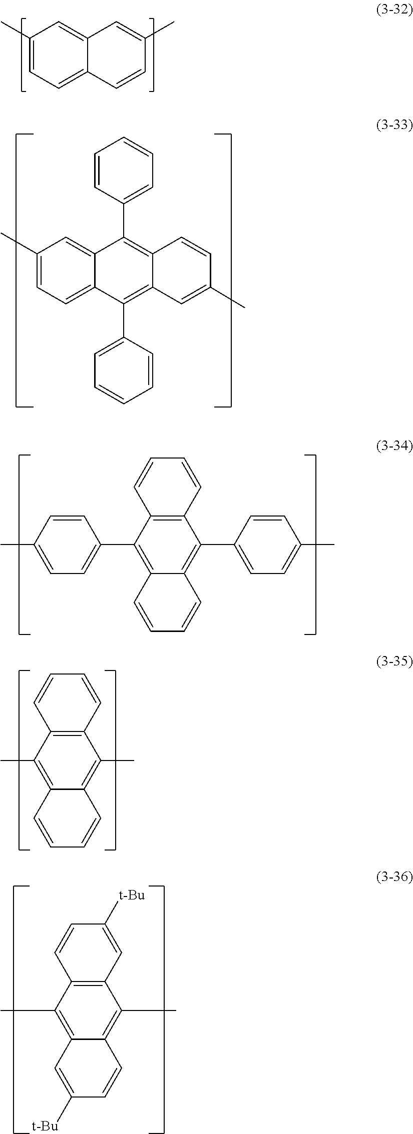

As the third constitutional unit, the constitutional unit represented by the following formula (5) (constitutional unit consisting of the group represented by the following formula (5′)) can be selected:

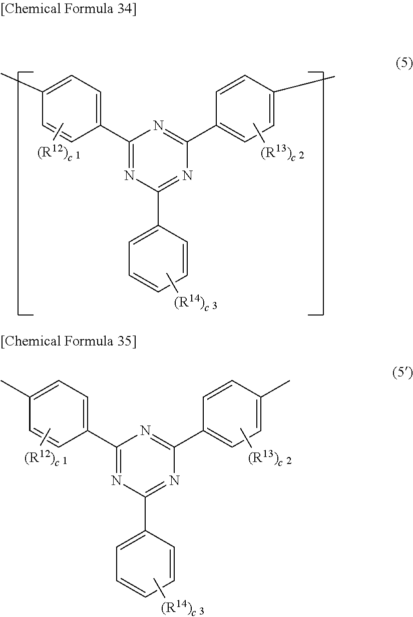

In the formula (5) and the formula (5′), c¹ and c² each independently represent an integer of 0 to 4; c³ represents an integer of 0 to 5. R¹², R¹³, and R¹⁴ each independently represent an unsubstituted or substituted alkyl group, an unsubstituted or substituted alkoxy group, an unsubstituted or substituted aryl group, an unsubstituted or substituted aryloxy group, an unsubstituted or substituted monovalent heterocyclic group, an unsubstituted or substituted alkoxycarbonyl group, an unsubstituted or substituted silyl group, a halogen atom, a carboxyl group, or a cyano group. When R¹², R¹³, and R¹⁴ exist in plural, the plurality of R¹², R¹³, or R¹⁴ may be the same or different from each other.

In the formula (5) and the formula (5′), it is preferable that c¹ and c² be an integer of 0 to 2, and c³ be an integer of 1 to 3 because the light emission efficiency of the light-emitting device using the polymer compound according to the present embodiment is more excellent.

In the formula (5) and the formula (5′), in the case where the group represented by R¹², R¹³, and R¹⁴ has a substituent, the substituent is preferably an alkyl group, an alkoxy group, an aryl group, an aryloxy group, an arylalkyl group, an arylalkoxy group, an arylalkenyl group, an arylalkynyl group, an amino group, a substituted amino group, a halogen atom, an acyl group, an acyloxy group, a heterocyclic group, a carboxyl group, a nitro group, and a cyano group, more preferably an alkyl group, an alkoxy group, an aryl group, an aryloxy group, an arylalkyl group, an arylalkoxy group, a substituted amino group, an acyl group, a cyano group, and still more preferably an alkyl group, an alkoxy group, and an aryl group.

In the formula (5) and the formula (5′), R¹², R¹³, and R¹⁴ can be, for example, a hydrogen atom, an unsubstituted or substituted alkyl group, an unsubstituted or substituted alkoxy group, or an unsubstituted or substituted aryl group. Here, examples of the substituted alkyl group in R¹², R¹³, and R¹⁴ include an arylalkyl group or an alkylarylalkyl group; examples of the substituted alkoxy group in R¹², R¹³, and R¹⁴ include an alkoxy group substituted by an arylalkoxy group or an alkoxy group; examples of the substituted aryl group in R¹², R¹³, and R¹⁴ include an alkylaryl group.

It is preferable that R¹², R¹³, and R¹⁴ be a hydrogen atom, an unsubstituted or substituted alkyl group, or an unsubstituted or substituted aryl group, and it is more preferable that R¹², R¹³, and R¹⁴ be an unsubstituted or substituted alkyl group or an unsubstituted or substituted aryl group because the light emission efficiency of the light-emitting device using the polymer compound according to the present embodiment is more excellent.

It is preferable that as the third constitutional unit, the polymer compound have a constitutional unit consisting of an unsubstituted or substituted fluorenediyl group, and it is more preferable that as the third constitutional unit, the polymer compound have a constitutional unit consisting of an unsubstituted or substituted 2,7-fluorenediyl group.

It is preferable that as the third constitutional unit, the polymer compound have a constitutional unit consisting of the group selected from the group consisting of an unsubstituted or substituted phenylene group, an unsubstituted or substituted naphthalenediyl group, an unsubstituted or substituted anthracenediyl group, and the group represented by the above formula (5′).

As the third constitutional unit, the polymer compound may have only one constitutional unit above, or may have a plurality of different constitutional units among the constitutional units above. The polymer compound may have the first constitutional unit, the second constitutional unit, the constitutional unit consisting of an unsubstituted or substituted fluorenediyl group, and the constitutional unit consisting of an unsubstituted or substituted phenylene group.

The polymer compound may have the first constitutional unit, the second constitutional unit, the constitutional unit consisting of an unsubstituted or substituted fluorenediyl group, and the constitutional unit consisting of an unsubstituted or substituted naphthalenediyl group.

The polymer compound may have the first constitutional unit, the second constitutional unit, the constitutional unit consisting of an unsubstituted or substituted fluorenediyl group, and the constitutional unit consisting of an unsubstituted or substituted anthracenediyl group.

The polymer compound may have the first constitutional unit, the second constitutional unit, the constitutional unit consisting of an unsubstituted or substituted fluorenediyl group, and the constitutional unit represented by the above formula (5).

It is preferable that the content (total content) of the third constitutional unit be 0.1 to 99.9 mol % of the total constitutional units, it is more preferable that the content (total content) of the third constitutional unit be 30 to 99.9 mol % of the total constitutional units, and it is still more preferable that the content (total content) of the third constitutional unit be 50 to 99.9 mol % of the total constitutional units because the light emission efficiency of the light-emitting device to be obtained is more excellent in the case where the polymer compound is used in production of the light-emitting device.

Examples of a combination of the constitutional units in the polymer compound are shown below:

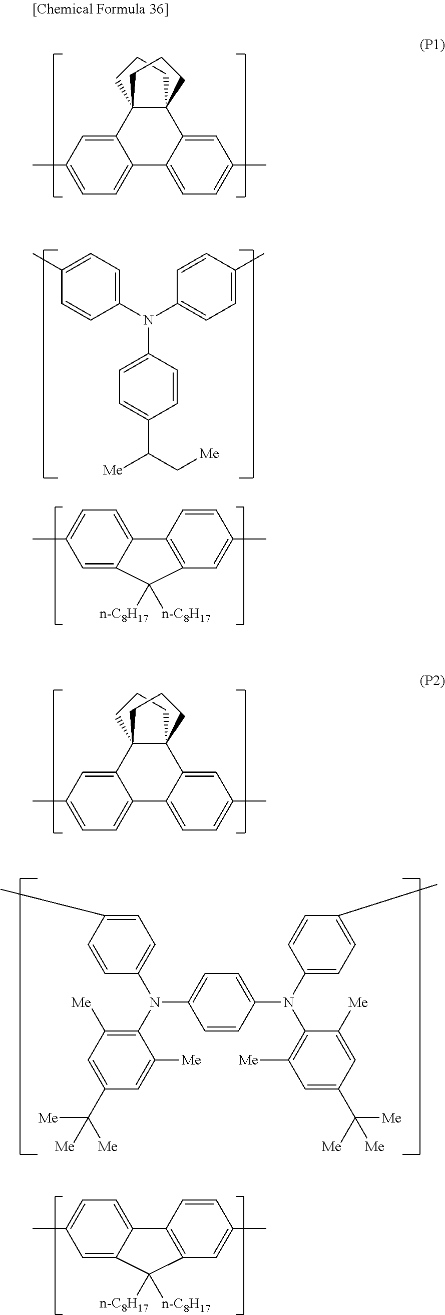

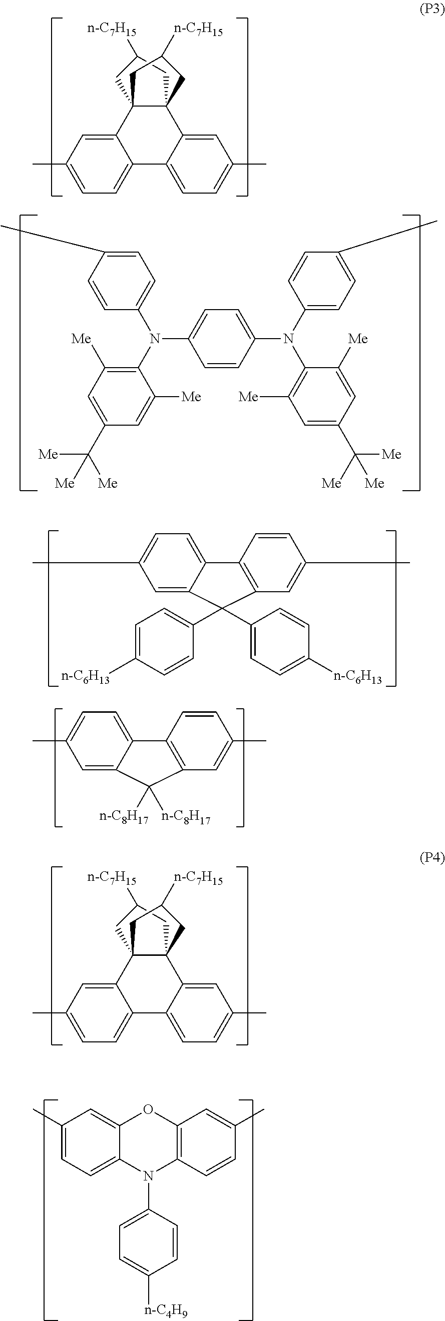

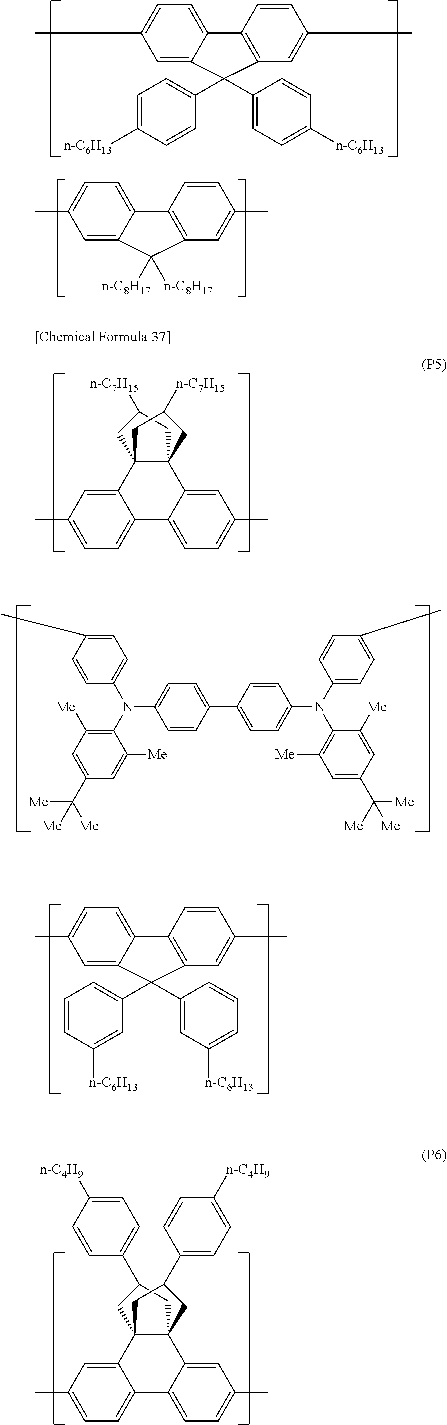

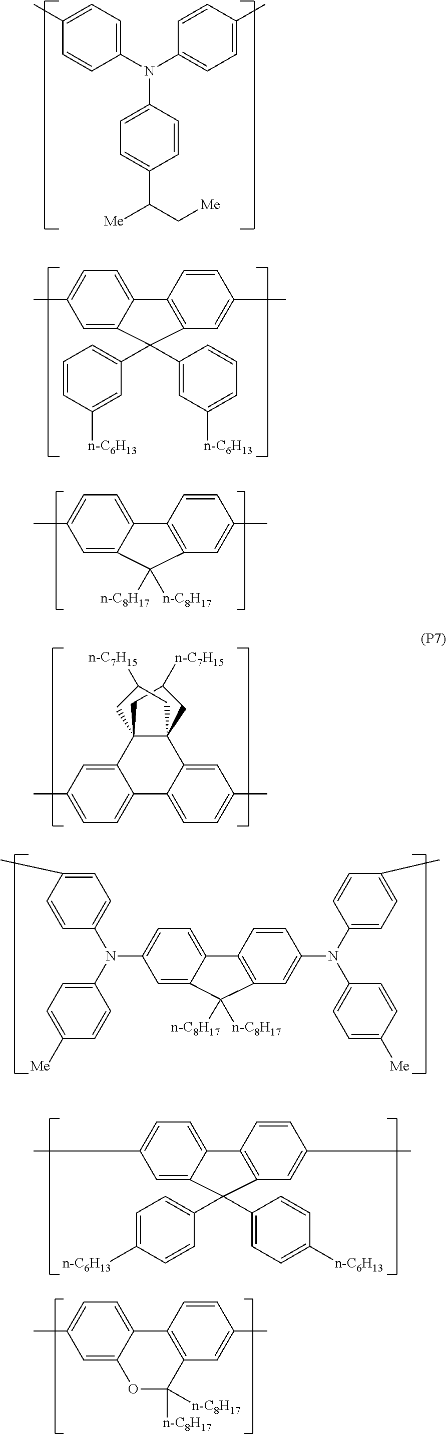

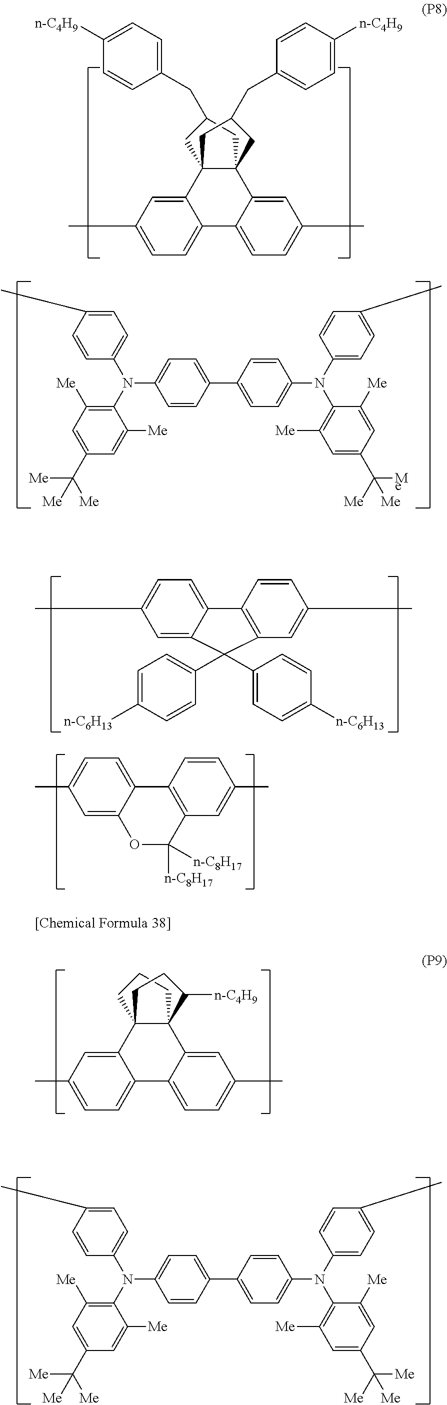

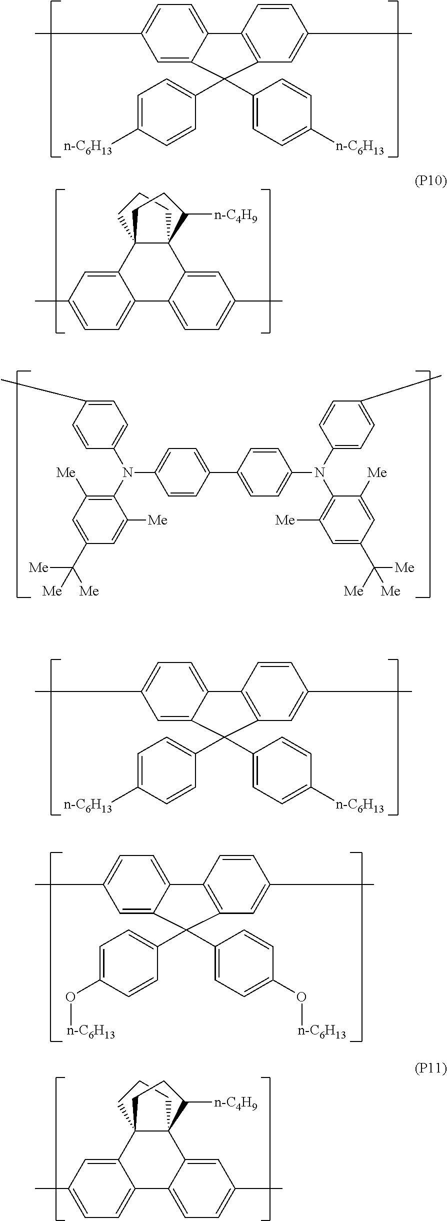

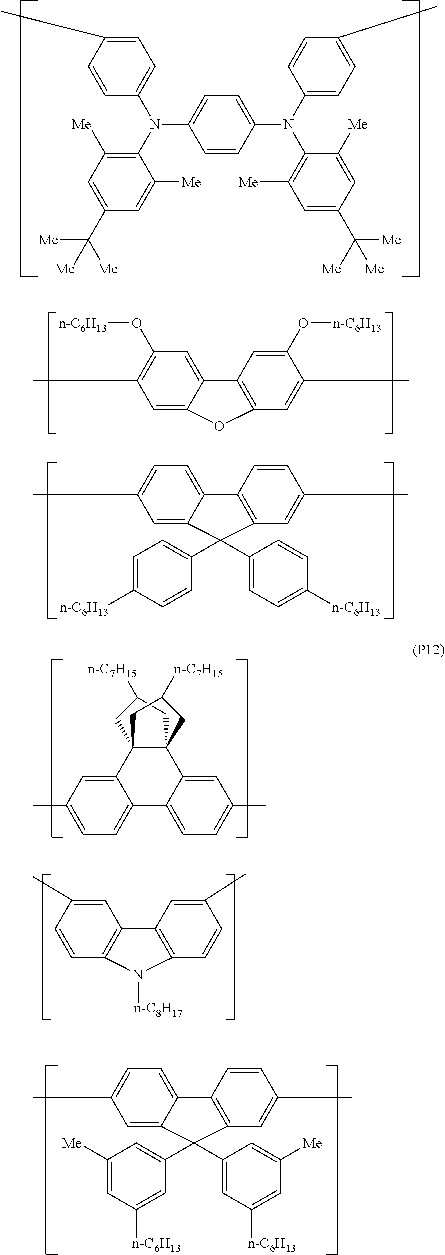

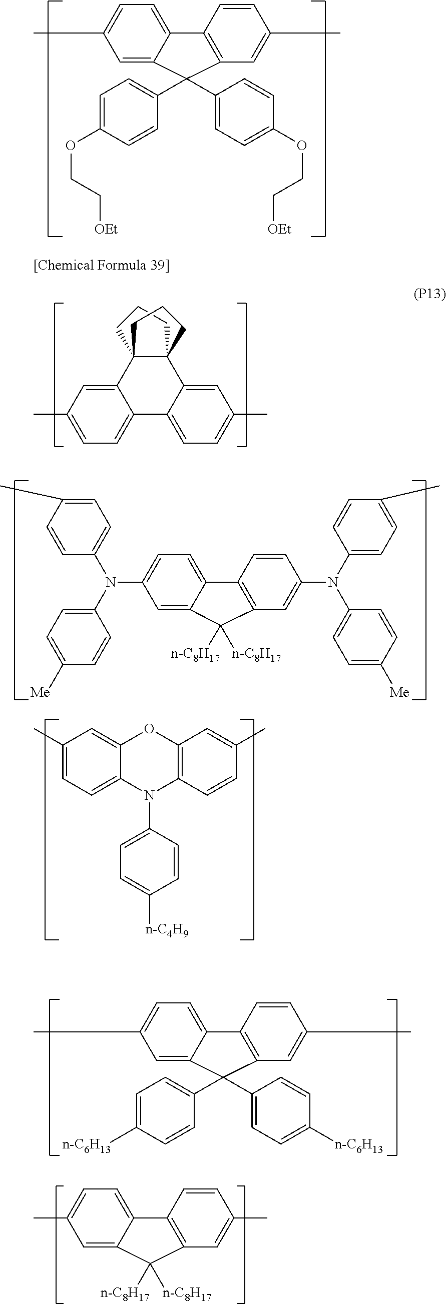

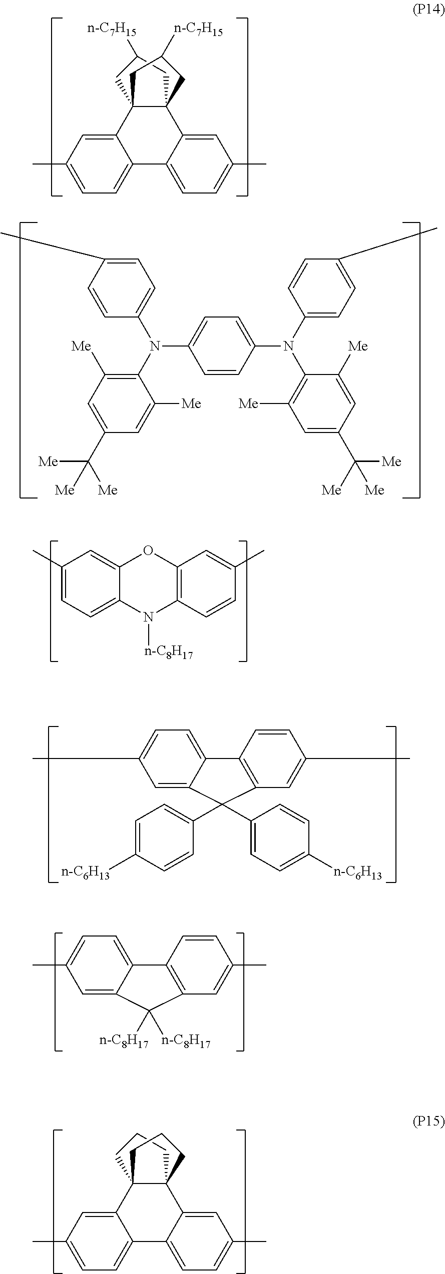

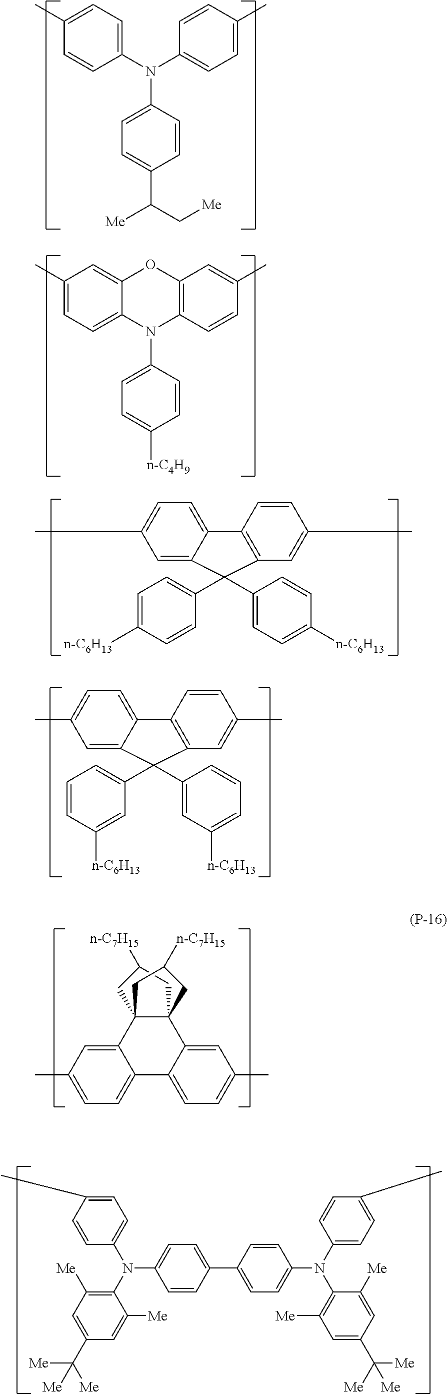

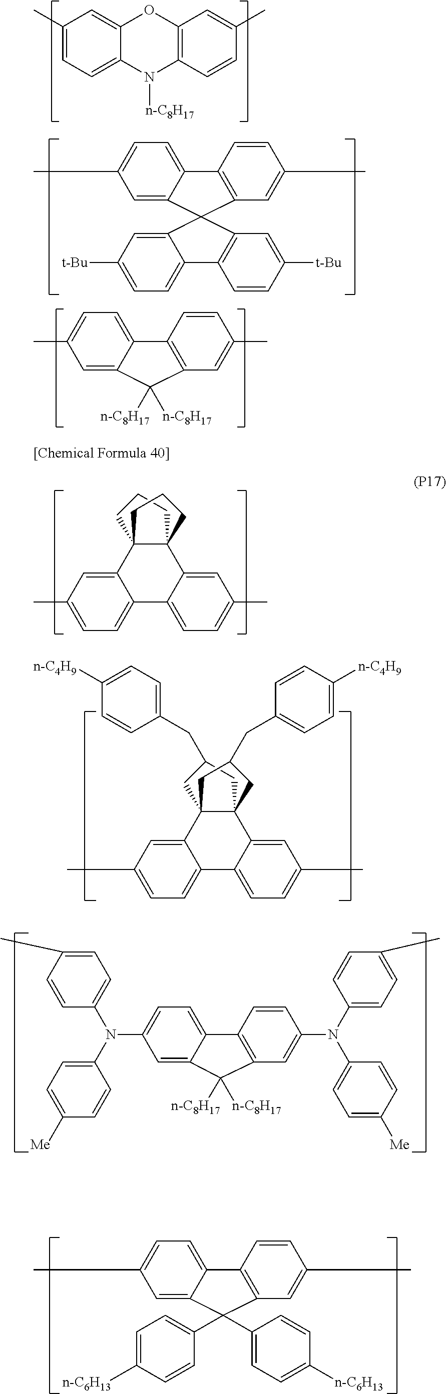

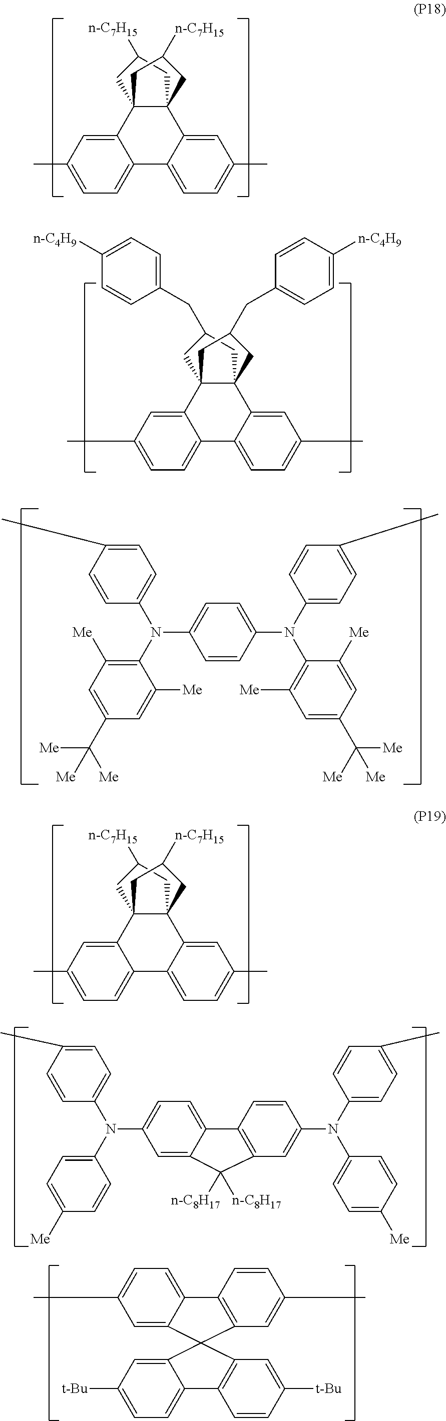

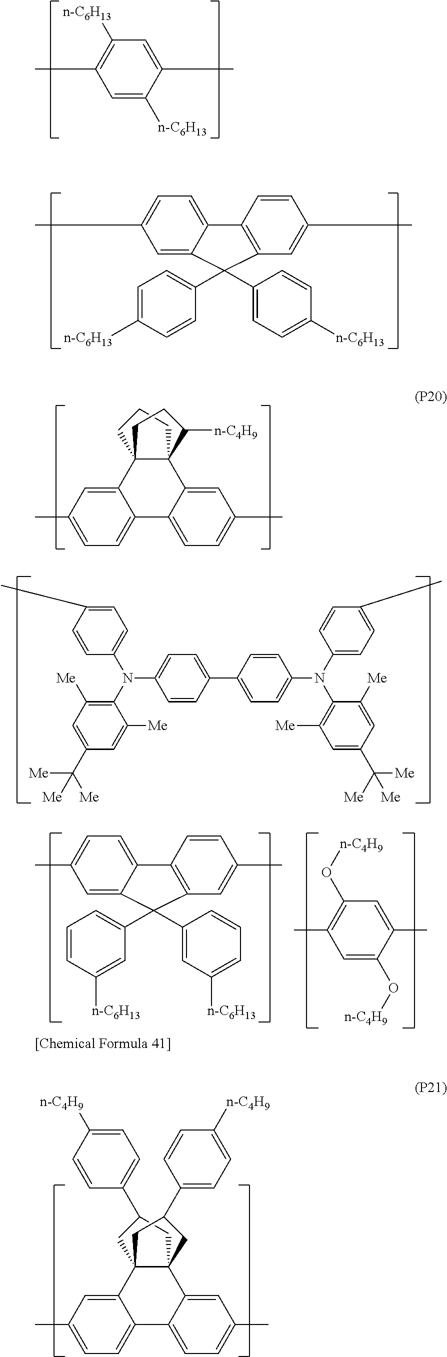

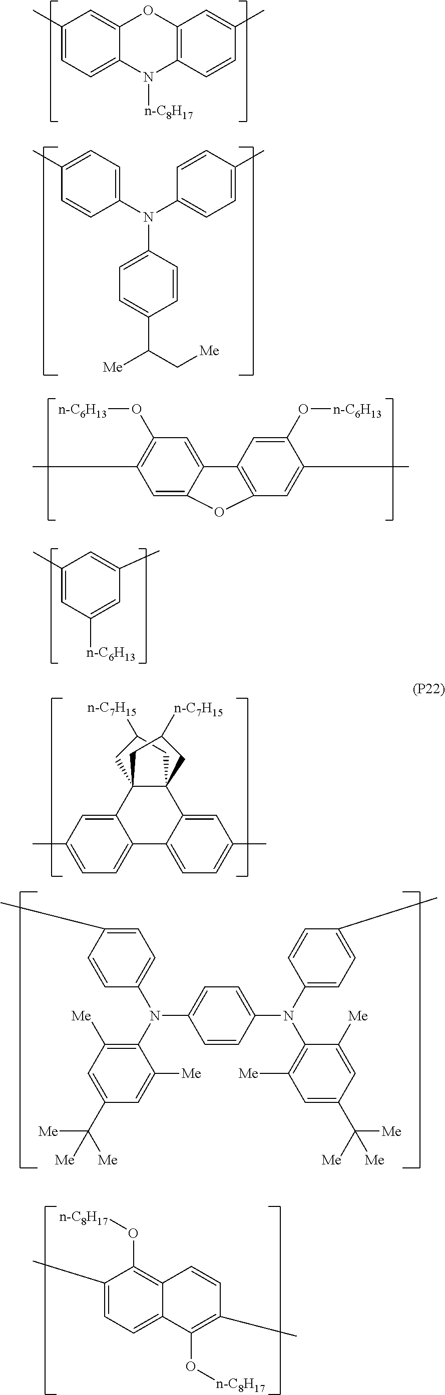

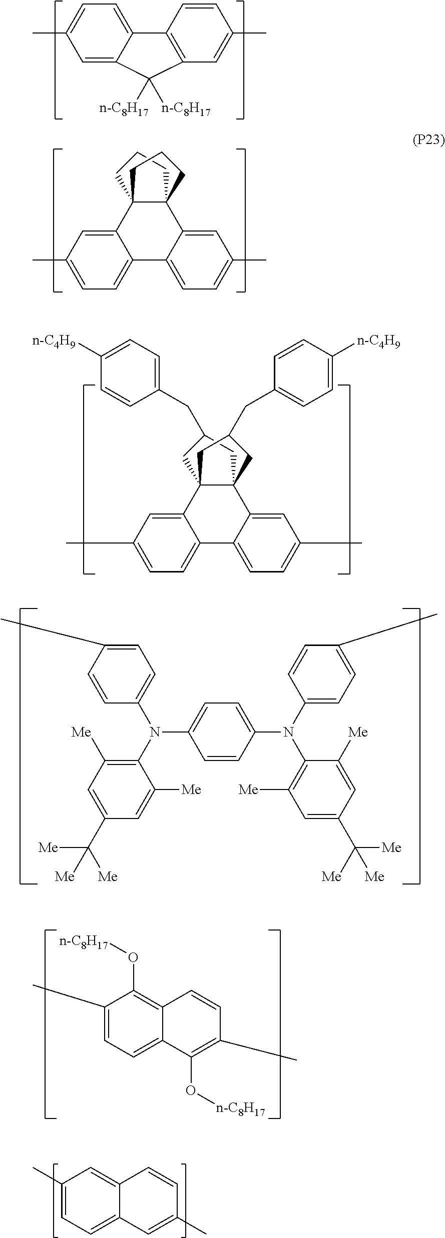

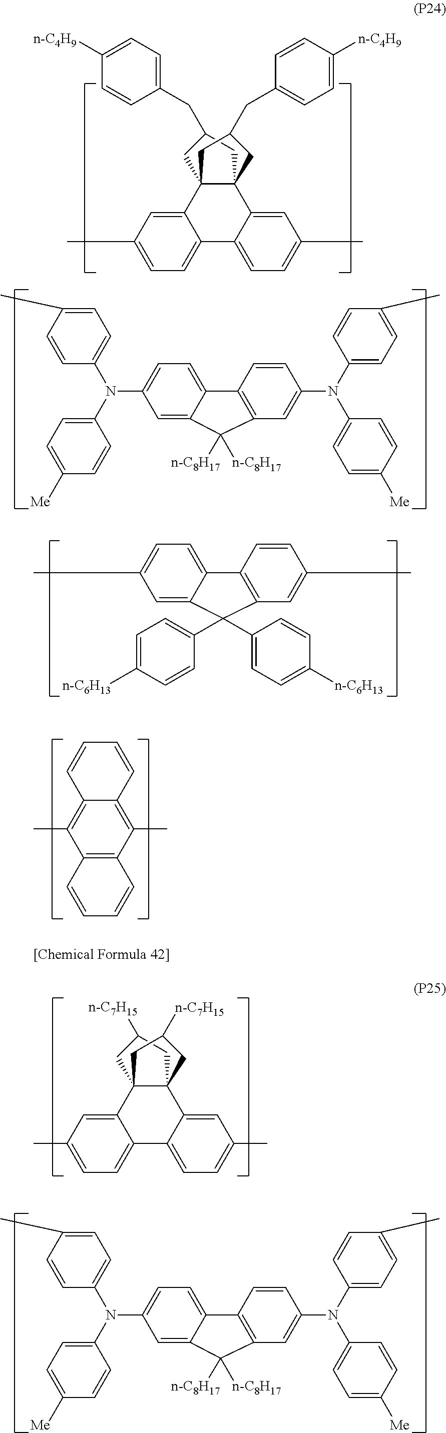

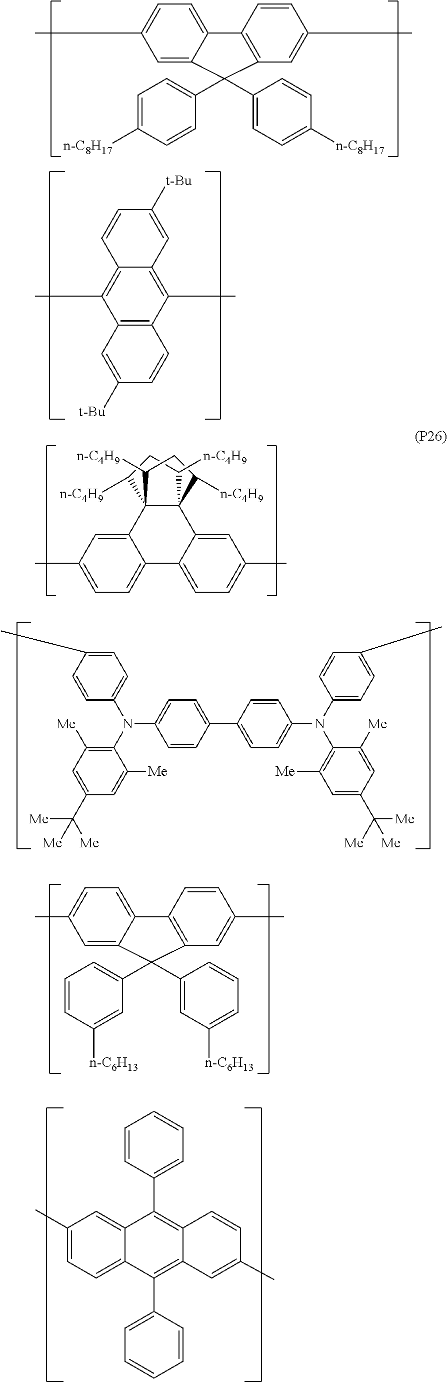

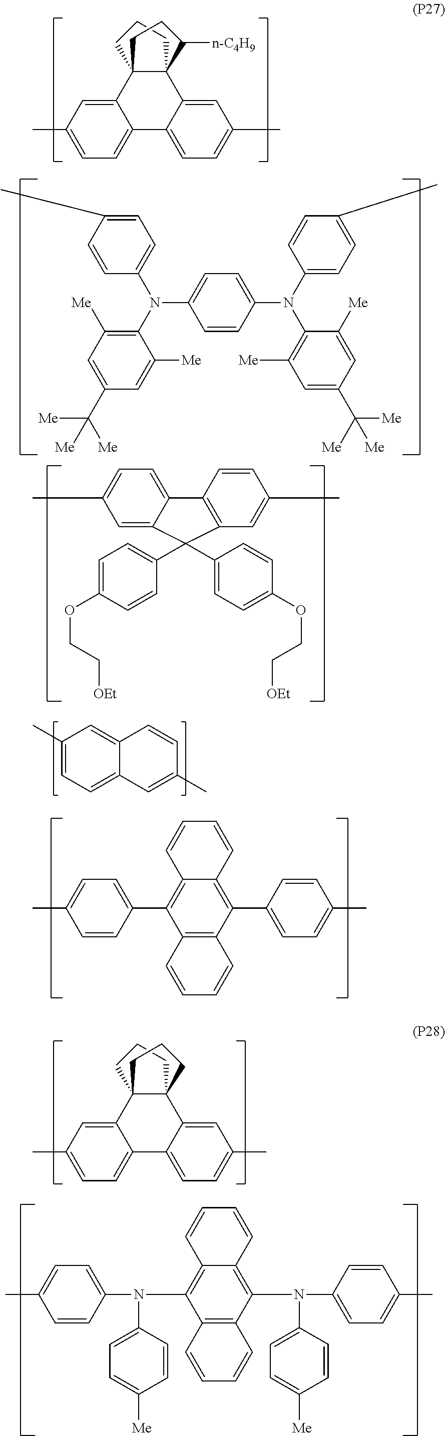

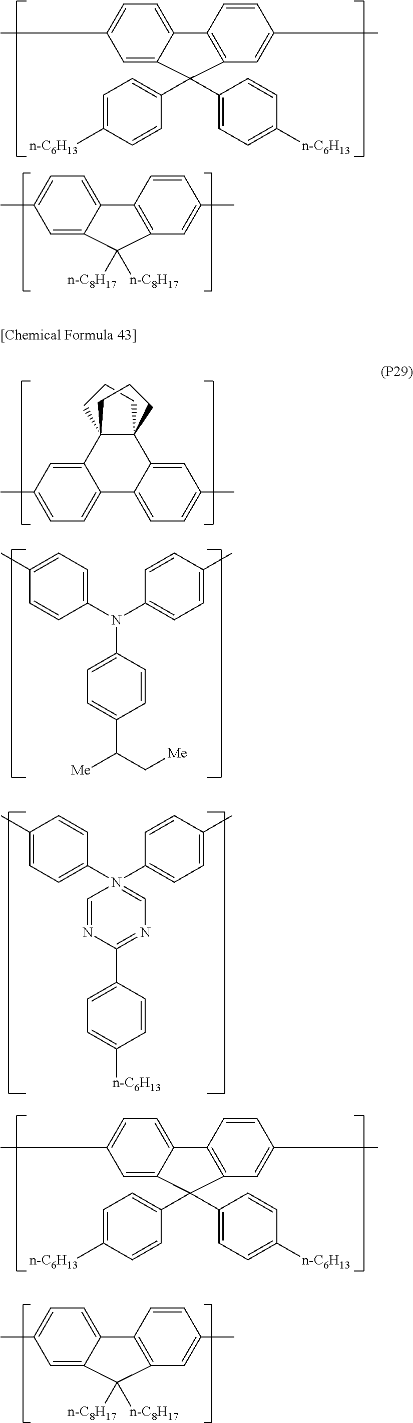

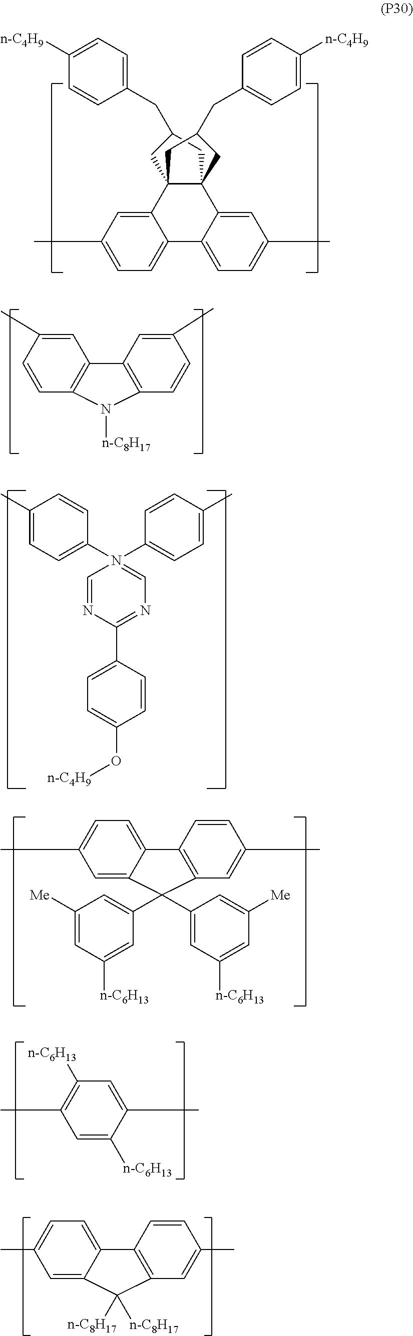

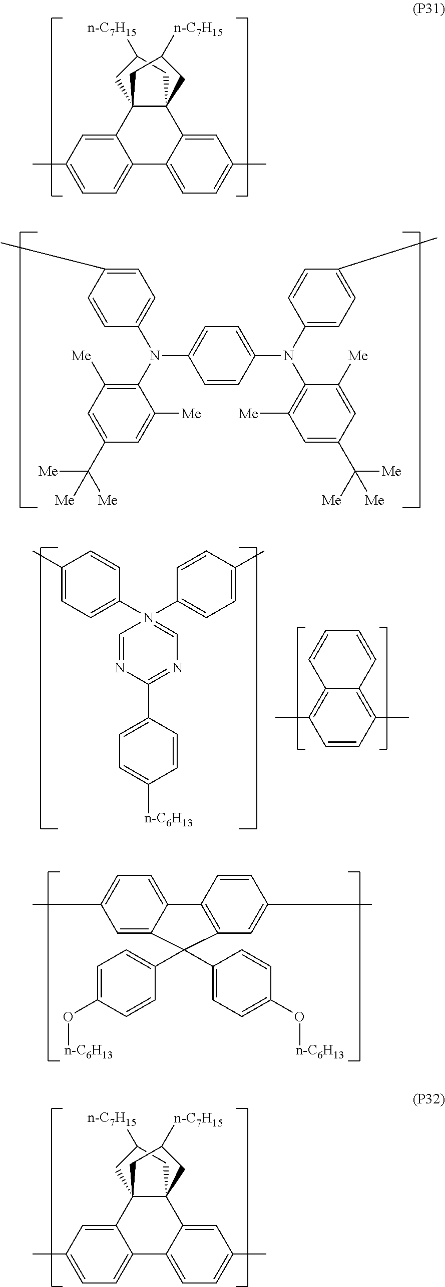

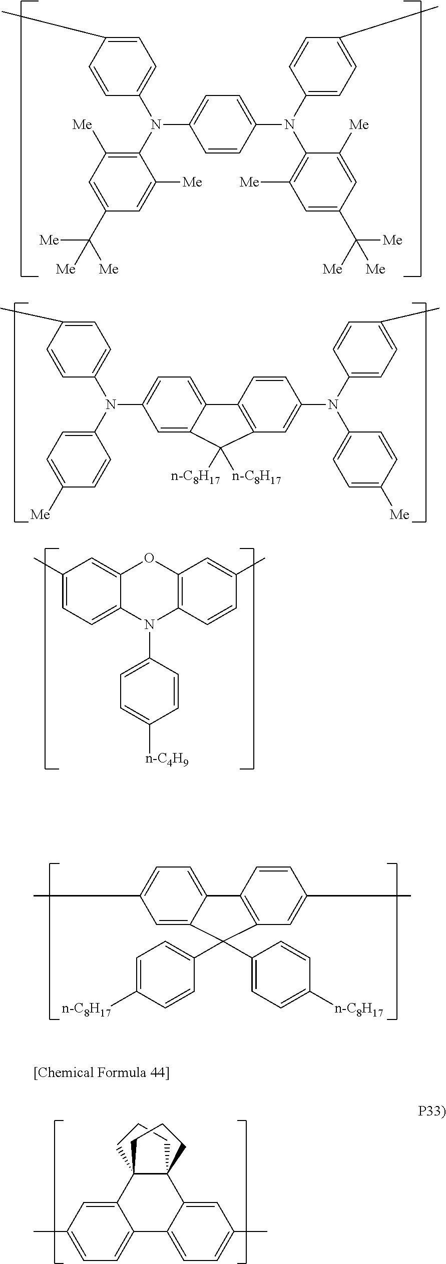

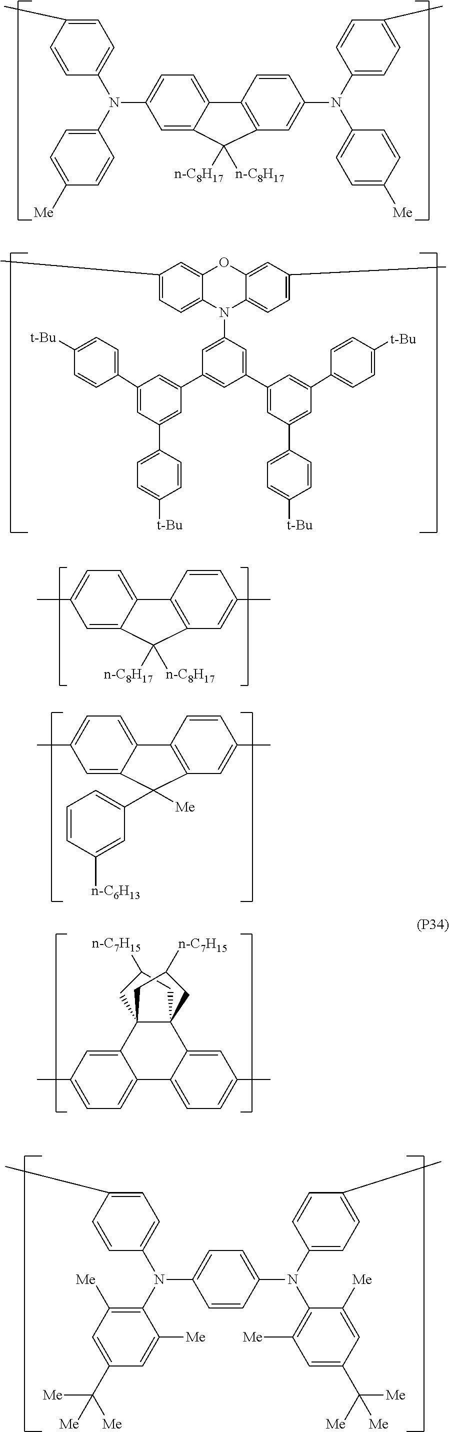

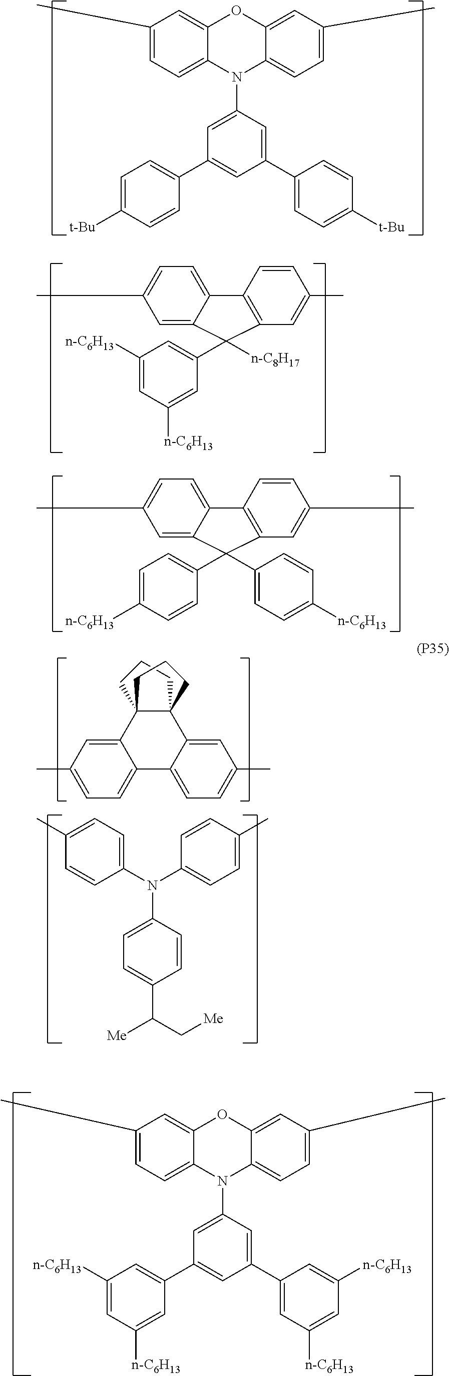

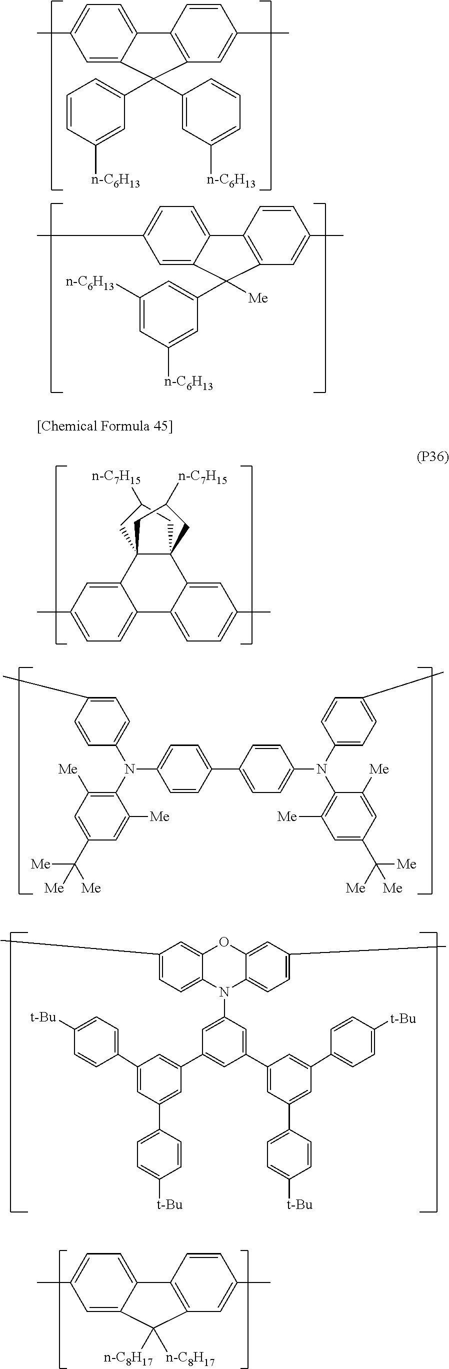

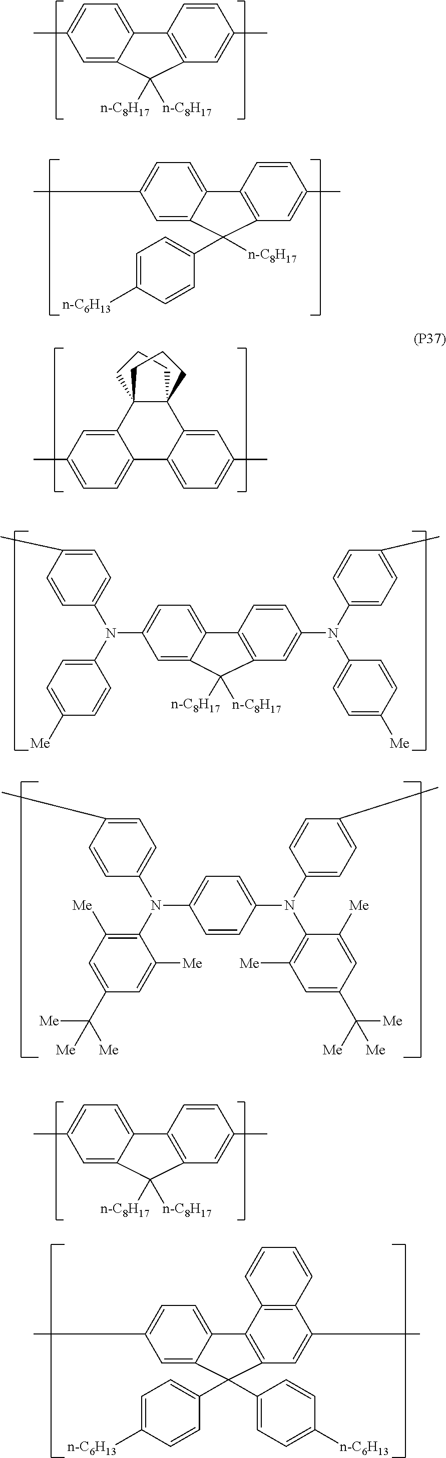

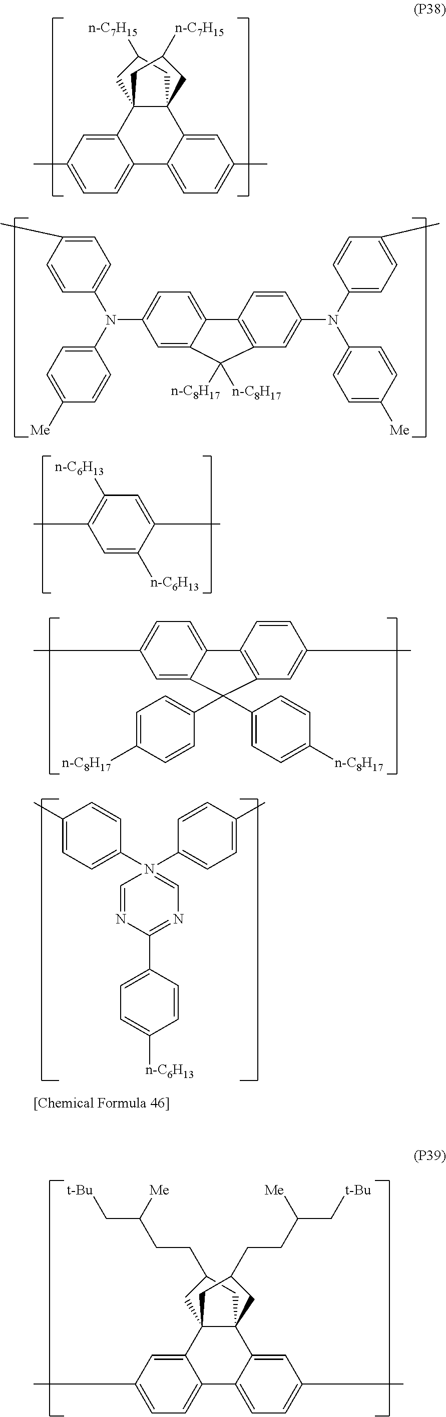

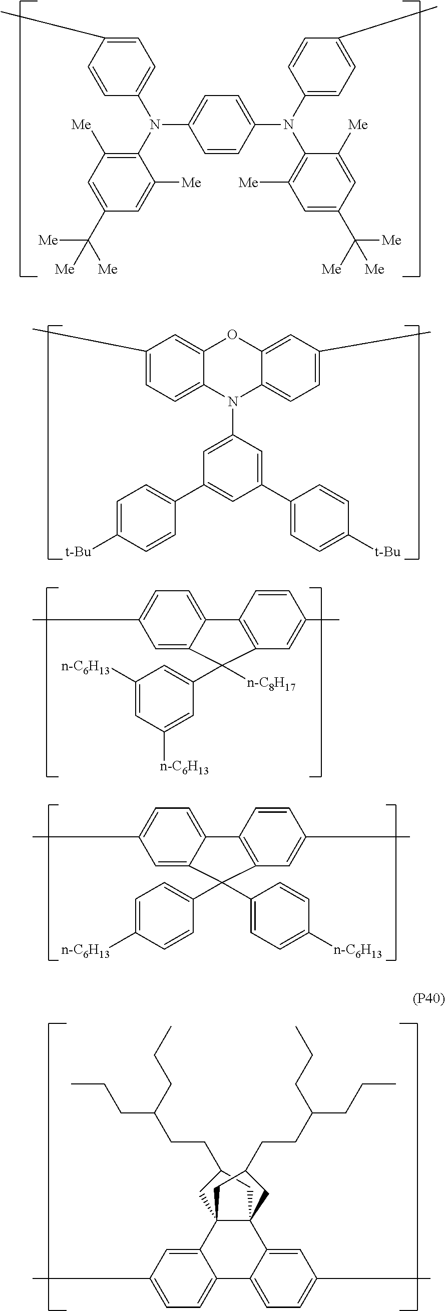

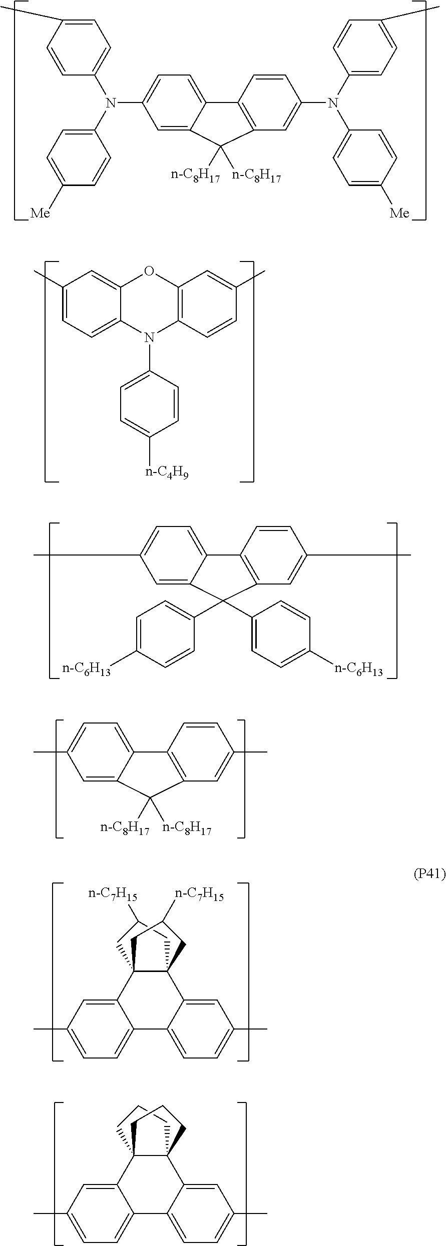

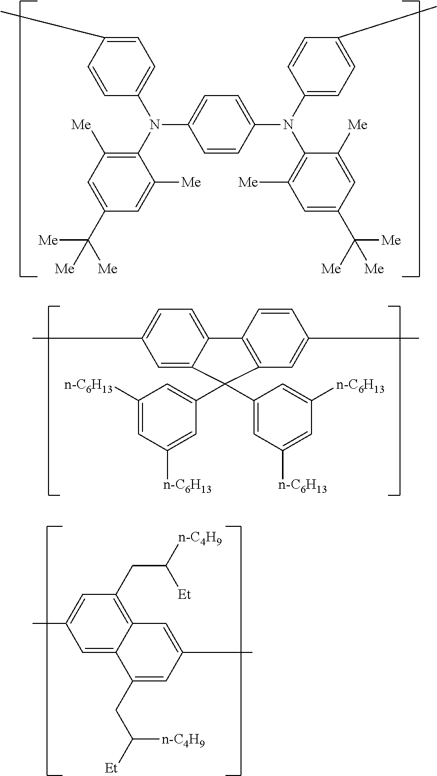

If a polymerizable group remains as it is in the terminal group, the polymer compound according to the present embodiment has a possibility of reducing the light emission properties and life of the light-emitting device produced using the polymer compound. For this reason, it is preferable that the terminal group be a stable group (such as an aryl group and a monovalent heterocyclic group (particularly, a monovalent aromatic heterocyclic group)).

The polymer compound according to the present embodiment may be any copolymer; for example, the polymer compound according to the present embodiment may be any of block copolymers, random copolymers, alternating copolymers, and graft copolymers.

The polymer compound according to the present embodiment is useful as light-emitting materials, charge transport materials, and the like, and may be used in combination with other compound as a composition described later.

The polystyrene-equivalent number-average molecular weight of the polymer compound according to the present embodiment measured by gel permeation chromatography (hereinafter, referred to as “GPC”) is preferably 1×10³ to 1×10⁷, and more preferably 1×10⁴ to 5×10⁶. The polystyrene-equivalent weight-average molecular weight of the polymer compound according to the present embodiment is preferably 1×10⁴ to 5×10⁷, and more preferably 5×10⁴ to 1×10⁷.

It is preferable that the glass transition temperature of the polymer compound according to the present embodiment be 70° C. or more because durability against various processes for producing the light-emitting device is high and the heat resistance of the light-emitting device is good.

The light-emitting device using the polymer compound is a high performance light-emitting device that can be derived with excellent light emission efficiency. Accordingly, the light-emitting device is useful for backlights of liquid crystal displays, curved or flat light sources for lighting, segment display devices, dot matrix display devices, and the like. Further, the polymer compound according to the present embodiment can also be used as a dye for a laser, a material for an organic solar cell, an organic semiconductor for an organic transistor, a material for a conductive film such as conductive films and organic semiconductor films, and a light-emittable film material that emits fluorescence or phosphorescence.

(Method for Producing Polymer Compound)

The polymer compound can be produced by condensation polymerizing the compound represented by the following formula (1M) (hereinafter, referred to as a “compound 1M” depending on cases) with the compound represented by the following formula (2M) (hereinafter, referred to as a “compound 2M” depending on cases). Herein, the compound 1M, the compound 2M, and a compound 4M described later are collectively referred to as the “monomer” in some cases.

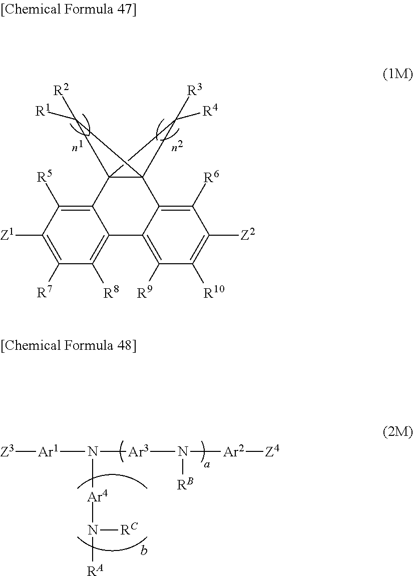

In the formula (1M), n¹, n², R¹, R², R³, R⁴, R⁵, R⁶, R⁷, R⁸, R⁹, and R¹⁰ are the same as above; Z¹ and Z² each independently represent a group selected from the following substituent groups (the following substituent group A or the following substituent group B).

In the formula (2M), a, b, Ar¹, Ar², Ar³, Ar⁴, R^{A}, and R^{B} are the same as above; Z³ and Z⁴ represent a group selected from the following substituent group A or the following substituent group B.

<Substituent Group A>

Groups represented by a chlorine atom, a bromine atom, an iodine atom, and —O—S(═O)₂R⁴¹ (R⁴¹ represents an alkyl group, or an aryl group which may be substituted by alkyl group, alkoxy group, nitro group, fluorine atom, or a cyano group).

<Substituent Group B>

Groups represented by —B(OR⁴²)₂ (R⁴² represents a hydrogen atom or an alkyl group; and a plurality of R⁴² present may be the same or different from each other and may be linked to each other to form a cyclic structure); groups represented by —BF₄Q¹ (Q¹ represents a monovalent cation selected from the group consisting of Li⁺, Na⁺, K⁺, Rb⁺, and Cs⁺); groups represented by —MgY¹ (Y¹ represents a chlorine atom, a bromine atom, or an iodine atom); groups represented by —ZnY² (Y² represents a chlorine atom, a bromine atom, or an iodine atom); and groups represented by —Sn (R⁴³)₃ (R⁴³ represents a hydrogen atom or an alkyl group; and a plurality of R⁴³ present may be the same or different from each other and may be linked to each other to form a cyclic structure).

It is known that the compound having the group selected from the substituent group A and the compound having the group selected from the substituent group B are condensation polymerized by a known coupling reaction, and carbon atoms bonded to the groups are bonded. For this reason, if the compound A having two groups selected from the substituent group A and the compound B having two groups selected from the substituent group B are fed to the known coupling reaction, a condensation polymer of the compound A and the compound B can be obtained by condensation polymerization.

A condensation polymer can also be obtained, for example, by a method for polymerizing the compound having two groups selected from the substituent group A with an Ni(0) catalyst (Yamamoto polymerization) (Progress in Polymer Science, Vol. 17, pp. 1153 to 1205, 1992).

In such a condensation polymerization, the first constitutional unit is derived from the compound 1M, and the second constitutional unit is derived from the compound 2M.

In the method for producing a polymer compound, a compound other than those described above may be fed to the condensation polymerization; for example, the compound represented by the following formula (4M) (hereinafter, referred to as a “compound 4M” depending on cases) can further be fed to the condensation polymerization. By feeding the compound 4M to the condensation polymerization, the third constitutional unit is introduced into the polymer compound to be obtained.

[Chemical Formula 49]

Z⁵—Ar⁵—Z⁶ (4M)

wherein Ar⁵ is the same as above; Z⁵ and Z⁶ represent the group selected from the substituent group A or the substituent group B. Z⁵ and Z⁶ can be selected according to the Z¹ and Z² in the compound 1M and Z³ and Z⁴ in the compound 2M.

Examples of the condensation polymerization method include a method for polymerization using the Suzuki coupling reaction (Chem. Rev.), Vol. 95, pp. 2457-2483 (1995)), a method for polymerization using the Grignard reaction (Bull. Chem. Soc. Jpn., Vol. 51, p. 2091 (1978)), a method for polymerization using an Ni(0) catalyst (Progress in Polymer Science, Vol. 17, pp. 1153 to 1205, 1992), and a method using the Stille coupling reaction (European Polymer Journal), Vol. 41, pp. 2923-2933 (2005)). Among these, from the viewpoint of easy synthesis of raw materials and simple operation of the polymerization reaction, the method for polymerization using the Suzuki coupling reaction and the method for polymerization using an Ni(0) catalyst are preferable; considering easy control of the structure of the polymer compound, a method for polymerization using an aryl-aryl cross coupling reaction such as the Suzuki coupling reaction, the Grignard reaction, and the Stille coupling reaction is more preferable; the reaction using the polymerization by the Suzuki coupling reaction is particularly preferable.

Examples of the condensation polymerization method include a method of reacting the compounds above with a proper catalyst or base when necessary. In the case where the method for polymerization using the Suzuki coupling reaction is selected, in order to obtain the polymer compound having a desired molecular weight, the ratio of the total mole number of the group selected from substituent group B that each compound has to the total mole number of the group selected from the substituent group A that each compound has may be adjusted. Usually, it is preferable that the ratio of the latter mole number to the former mole number be 0.95 to 1.05, it is more preferable that the ratio of the latter mole number to the former mole number be 0.98 to 1.02, and it is still more preferable that the ratio of the latter mole number to the former mole number be 0.99 to 1.01.

It is preferable that the amount of the compound 1M to be used in the condensation polymerization be 0.5 mol % or more based on the total molar amount of the compound 1M and other monomer, it is more preferable that the amount of the compound 1M to be used in the condensation polymerization be 0.5 to 80 mol % based on the total molar amount of the compound 1M and other monomer, and it is still more preferable that the amount of the compound 1M to be used in the condensation polymerization be 5 to 60 mol % based on the total molar amount of the compound 1M and other monomer. It is preferable that the amount of the compound 2M to be used in the condensation polymerization be 0.1 mol % or more based on the total molar amount of the compound 2M and other monomer, it is more preferable that the amount of the compound 2M to be used in the condensation polymerization be 0.1 to 50 mol % based on the total molar amount of the compound 2M and other monomer, and it is still more preferable that the amount of the compound 2M to be used in the condensation polymerization be 0.1 to 40 mol % based on the total molar amount of the compound 2M and other monomer. By using the polymer compound obtained by such a condensation polymerization, the light-emitting device can be produced.

The monomer synthesized in advance and separated may be used, or the monomer may be synthesized during the reaction system and used as it is. In the case where the polymer compound to be obtained is used for the light-emitting device, the purity may affect the performance of the light-emitting device. For this reason, it is preferable that these monomers be refined by a method such as distillation, chromatography, sublimation refining, recrystallization or a combination thereof.

In the method of producing the polymer compound according to the present embodiment, it is preferable that the monomers be polymerized in the presence of a catalyst. In the case where polymerization is performed using the Suzuki coupling reaction, examples of the catalyst include transition metal complexes such as palladium complexes such as palladium[tetrakis(triphenylphosphine)], [tris(dibenzylideneacetone)]dipalladium, palladium acetate, and dichlorobistriphenylphosphinepalladium; and complexes in which a ligand such as triphenylphosphine, tri-tert-butylphosphine, and tricyclohexylphosphine is coordinated with these transition metal complexes.

In the case where the polymerization is performed using the Ni(0) catalyst, examples of the Ni(0) catalyst include transition metal complexes such as nickel complexes such as nickel[tetrakis(triphenylpho sphine)], [1,3-bis(diphenylphosphino)propane]dichloronickel, [bis(1,4-cyclooctadiene)]nickel; and complexes in which a ligand such as triphenylphosphine, tri-tert-butylphosphine, tricyclohexylphosphine, diphenylphosphinopropane, a substituted or unsubstituted bupyridyl, and a substituted or unsubstituted phenanthroline is coordinated with these transition metal complexes.

The catalyst synthesized in advance may be used, or the catalyst prepared during the reaction system may be used as it is. These catalysts may be used alone or in combination.

The amount of the catalyst may be an effective amount as the catalyst; for example, the amount in terms of the mole number of the transition metal is usually 0.0001 to 300 mol %, preferably 0.001 to 50 mol %, and more preferably 0.01 to 20 mol % based on 100 mol % of the total of all the monomers in the polymerization reaction.

In the method for polymerization using the Suzuki coupling reaction, it is preferable that a base be used. Examples of the base include inorganic salt groups such as sodium carbonate, potassium carbonate, cesium carbonate, potassium fluoride, cesium fluoride, and tripotassium phosphate; and organic bases such as tetrabutylammonium fluoride, tetrabutylammonium chloride, tetrabutylammonium bromide, tetraethylammonium hydroxide, and tetrabutylammonium hydroxide.

The amount of the base is usually 50 to 2000 mol %, and preferably 100 to 1000 mol % based on 100 mol % of the total of all the monomers in the polymerization reaction.

The polymerization reaction may be performed in the absence of a solvent, or performed in the presence of a solvent; usually, the polymerization reaction is performed in the presence of an organic solvent. Here, examples of the organic solvent include toluene, xylene, mesitylene, tetrahydrofuran, 1,4-dioxane, dimethoxyethane, N,N-dimethylacetoamide, and N,N-dimethylformamide. Usually, in order to suppress a side reaction, it is desired that a solvent subjected to a deoxidation treatment be used. The organic solvents may be used alone or in combination.

It is preferable that the amount of the organic solvent to be used be an amount such that the total concentration of all the monomers in the polymerization reaction is 0.1 to 90% by weight, it is more preferable that the amount of the organic solvent to be used be an amount such that the total concentration of all the monomers in the polymerization reaction is 1 to 50% by weight, and it is still more preferable that the amount of the organic solvent to be used be an amount such that the total concentration of all the monomers in the polymerization reaction is 2 to 30% by weight.

The reaction temperature of the polymerization reaction is preferably −100 to 200° C., more preferably −80 to 150° C., and still more preferably 0 to 120° C. The reaction time is usually 1 hour or more, and preferably 2 to 500 hours.

In the polymerization reaction, to avoid remaining of the polymerizable group (such as Z¹, Z²) at the terminal in the polymer compound according to the present embodiment, a compound represented by the following formula (IT) may be used as a chain-terminating agent. Thereby, a polymer compound whose terminal is the aryl group or monovalent heterocyclic group (particularly, the monovalent aromatic heterocyclic group) can be obtained.

Z^{T}—Ar^{T} (1 T)

wherein Ar^{T} represents an aryl group that may have a substituent or a monovalent heterocyclic group (particularly, a monovalent aromatic heterocyclic group) that may have a substituent; Z^{T} represents the group selected from the substituent group A and the substituent group B above. Examples of the aryl group and the monovalent heterocyclic group (particularly, the monovalent aromatic heterocyclic group) in Ar^{c} can include the aryl groups and monovalent heterocyclic groups (particularly, the monovalent aromatic heterocyclic groups) exemplified as R¹ above.

A post-treatment in the polymerization reaction can be performed by a known method; for example, a method of removing water-soluble impurities by separation of a solution, a method in which a precipitate obtained by adding the reaction solution after the polymerization reaction to a lower alcohol such as methanol is filtered and dried, and the like can be used alone or in combination.

In the case where the purity of the polymer compound according to the present embodiment is low, refining may be performed by the standard method such as recrystallization, reprecipitation, continuous extraction with a Soxhlet extractor, and column chromatography; in the case where the polymer compound according to the present embodiment is used for the light-emitting device, the purity may affect the performance of the light-emitting device such as light emission properties; for this reason, it is preferable that after the condensation polymerization, a purifying treatment such as reprecipitation refining and separation by chromatography be performed.

(Compound)

The compound according to the present embodiment is a compound represented by the following formula (6):

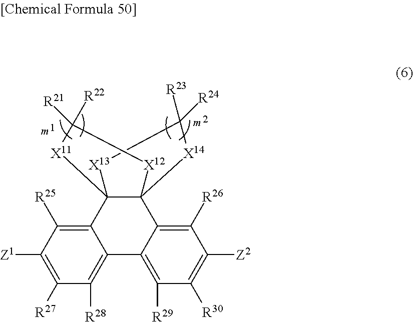

wherein m¹ and m² each independently represent 1 or 2; R²¹, R²², R²³, and R²⁴ are the same as R¹ to R⁴ above; X¹¹, X¹², X¹³, and X¹⁴ each independently represent a group represented by —C(R³¹)₂—. Here, R³¹ is the same as R¹, R², R³ and R⁴ above, and a plurality of R³¹ present may be the same or different from each other. R²⁵, R²⁶, R²⁷, R²⁸, R²⁹, and R³⁰ each are the same as R⁵, R⁶, R⁷, R⁸, R⁹, and R¹⁰; among R²⁷, R²⁸, R²⁹, and R³⁰, adjacent groups may be linked to each other to form a cyclic structure. Z¹ and Z² each independently represent a group selected from the substituent group (the substituent group A and the substituent group B). Among R²¹, R²², R²³, and R²⁴, at least one is a group other than a hydrogen atom.

In the formula (6), when R²¹ and R²² are different from each other or R²³ and R²⁴ are different from each other, a stereoisomer (diastereoisomer and/or enantiomer) may be present in the compound represented by the formula (6). The compound represented by the formula (6) may be a single stereoisomer, or may be a mixture of different stereoisomers.

Hereinafter, a method of producing the compound represented by the formula (6) will be described using an example in which m¹ and m² are 1. The compound represented by the formula (6) can be produced by the methods according to the following Schemes 1 to 5.

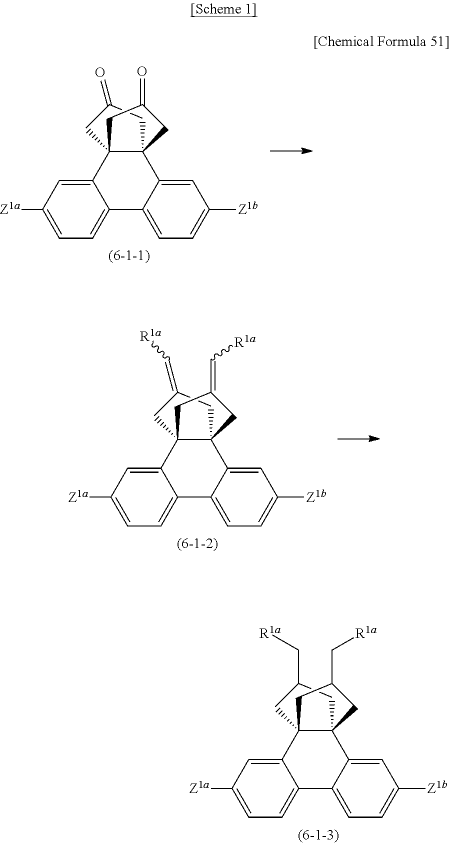

wherein the wavy line indicates that the compound having the wavy line is a geometric isomer mixture.

In Scheme 1, Z^{1a} and Z^{1b} each independently represent hydrogen atom or substituent group (the group selected from the substituent group A); R^{1a} represents an unsubstituted or substituted alkyl group, an unsubstituted or substituted aryl group, or an unsubstituted or substituted monovalent heterocyclic group. A plurality of R^{1a} present may be the same or different from each other.

In Scheme 1, first, by feeding the compound represented by the formula (6-1-1) (hereinafter, referred to as a “compound (6-1-1).”Hereinafter, the same is true of the compound represented by the formula (6-1-2)) to the Wittig reaction, the Horner-Wadsworth-Emmons reaction, or the like, a compound (6-1-2) is obtained. Next, by feeding the compound (6-1-2) to the reduction reaction, a compound (6-1-3) is obtained.

In the case where the Z^{1a} and Z^{1b} in the compound (6-1-3) are a hydrogen atom, by feeding the compound (6-1-3) to a reaction such as a bromination reaction, the hydrogen atom can be converted to the group selected from the substituent group A. In the case where the Z^{1a} and Z^{1b} in the compound (6-1-3) are the group selected from the substituent group A, the group can be converted to the group selected from the substituent group B by a known reaction.

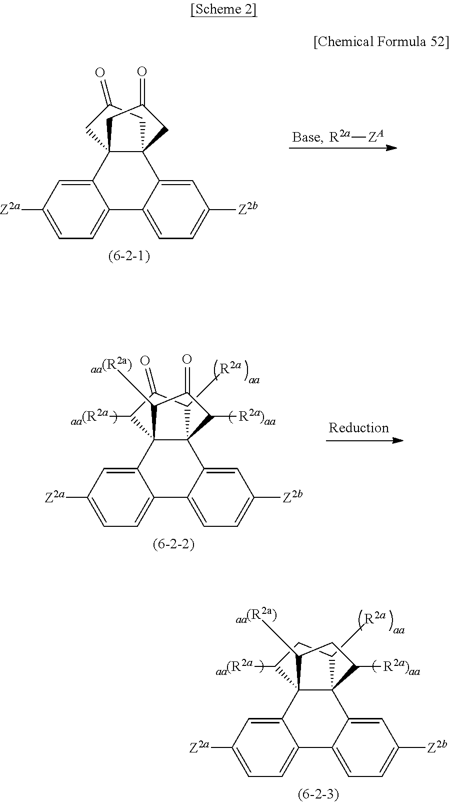

In Scheme 2, aa represents 0 or 1; Z^{2a} and Z^{2b} each independently represent a hydrogen atom or the group selected from the substituent group A; Z^{A} represents the group selected from the substituent group A; R^{2a} represents an unsubstituted or substituted alkyl group, an unsubstituted or substituted aryl group, or an unsubstituted or substituted monovalent heterocyclic group. A plurality of aa present may be the same or different from each other. In the case where a plurality of R^{ea} are present, those may be the same or different.

In Scheme 2, first, in the presence of a base, a compound (6-2-2) is obtained by an addition reaction of the compound (6-2-1) and R^{2a}—Z^{A}. Next, by feeding the compound (6-2-2) to the reduction reaction, a compound (6-2-3) is obtained.

In the case where Z^{2a} and Z^{2b} in the compound (6-2-3) are a hydrogen atom, by feeding the compound (6-2-3) to the reaction such as the bromination reaction, the hydrogen atom can be converted to the group selected from the substituent group A. In the case where Z^{2a} and Z^{2b} in the compound (6-2-3) are the group selected from the substituent group A, the group can be converted to the group selected from the substituent group B by a known reaction.

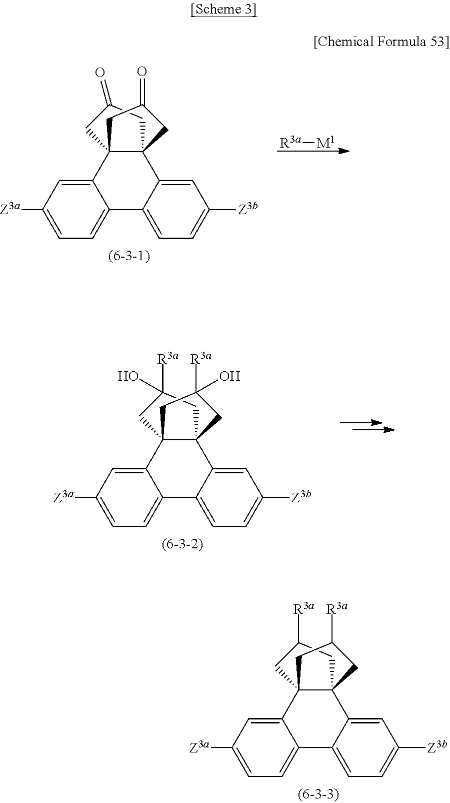

In Scheme 3, Z^{3a} and Z^{3b} each independently represent a hydrogen atom or the group selected from the substituent group A; R^{3a} represents an unsubstituted or substituted alkyl group, an unsubstituted or substituted aryl group, or an unsubstituted or substituted monovalent heterocyclic group. M¹ represents an alkali metal such as lithium and potassium or a group represented by -M^{II}Z^{H}; M^{II} represents Mg or Zn; Z^{H} represents a halogen atom. A plurality of R^{3a} present may be the same or different from each other.

In Scheme 3, first, a compound (6-3-2) is obtained by a reaction of the compound (6-3-1) with R^{3a}-M¹. Next, a compound (6-3-3) is obtained by converting a hydroxyl group to a hydrogen atom in the compound (6-3-2) by a known reaction.

In the case where Z^{3a} and Z^{3b} in the compound (6-3-3) are a hydrogen atom, by feeding the compound (6-3-3) to the reaction such as the bromination reaction, the hydrogen atom can be converted to the group selected from the substituent group A. In the case where Z^{3a} and Z^{3b} in the compound (6-3-3) are the group selected from the substituent group A, the group can be converted to the group selected from the substituent group B by a known reaction.

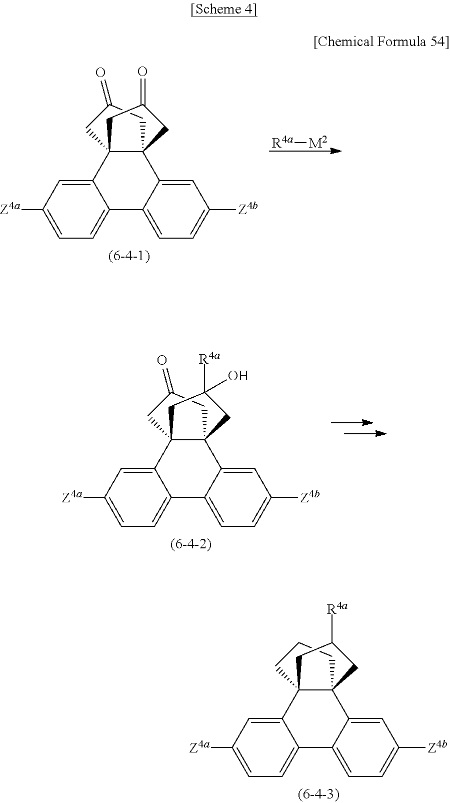

In Scheme 4, Z^{4a} and Z^{4b} each independently represent hydrogen atom or the group selected from the substituent group A; R^{4a} represents an unsubstituted or substituted alkyl group, an unsubstituted or substituted aryl group, or an unsubstituted or substituted monovalent heterocyclic group. M² represents an alkali metal such as lithium and potassium or a group represented by -M^{II}Z^{H}; M^{II} representsMg or Zn; Z^{H} represents a halogen atom.

In Scheme 4, first, a compound (6-4-2) is obtained by a reaction of the compound (6-4-1) with R^{4a}-M². Next, by feeding the compound (6-4-2) to the reduction reaction, a compound (6-4-3) is obtained.

In the case where Z^{4a} and Z^{4b} in the compound (6-4-3) are a hydrogen atom, by feeding the compound (6-4-3) to the reaction such as the bromination reaction, the hydrogen atom can be converted to the group selected from the substituent group A. In the case where Z^{4a} and Z^{4b} in the compound (6-4-3) are the group selected from the substituent group A, the group can be converted to the group selected from the substituent group B by a known reaction.

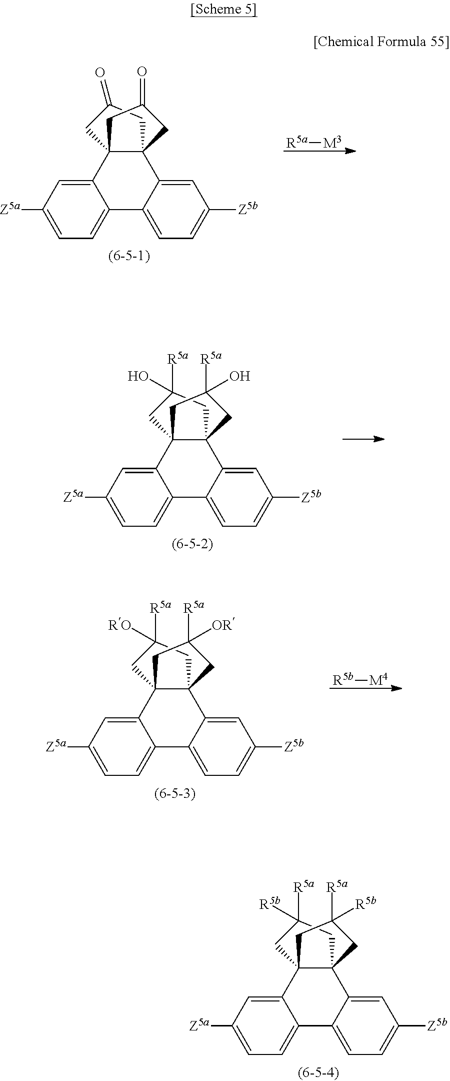

In Scheme 5, Z^{5b} and Z^{5b} each independently represent a hydrogen atom or the group selected from the substituent group A; R^{5a} and R^{5b} each independently represent unsubstituted or substituted alkyl group, an unsubstituted or substituted aryl group or unsubstituted or substituted monovalent heterocyclic group; R′ represents an unsubstituted or substituted alkyl group, an unsubstituted or substituted aryl group, or an unsubstituted or substituted monovalent heterocyclic group; M³ and M⁴ each independently represent alkali metal such as lithium and potassium or a group represented by —MgZ^{H}; Z^{H} represents a halogen atom. A plurality of R^{5a} present may be the same or different, and a plurality of R^{5b} present may be the same or different.

In Scheme 5, first, a compound (6-5-2) is obtained by a reaction of the compound (6-5-1) with R^{5a}-M³. Next, by feeding the compound (6-5-2), for example, to a reaction such as methanesulfonylation, a compound (6-5-3) having a leaving group is obtained. The compound (6-5-3) may be further reacted with R^{5b}-M⁴; by the reaction, a compound (6-5-4) is obtained.

In the case where Z^{5a} and Z^{5b} in the compound (6-5-3) and the compound (6-5-4) are a hydrogen atom, by feeding the compound (6-5-3) to the reaction such as the bromination reaction, the hydrogen atom can be converted to the group selected from the substituent group A. In the case where Z^{5a} and Z^{5b} in the compound (6-5-3) are the group selected from the substituent group A, the group can be converted to the group selected from the substituent group B by a known reaction.

The compound having a stereoisomer can be synthesized by performing a hydrogenation reaction (hydrogenating reaction) stereoselectively in Scheme 1 above as a method for obtaining a specific stereoisomer. Moreover, the specific stereoisomer can be condensed and refined by preferential crystallization. Besides, after the stereoisomer mixture is synthesized, the specific stereoisomer can be separated and refined by chromatography.

The compound (6-1-1), the compound (6-2-1), the compound (6-3-1), the compound (6-4-1), and the compound (6-5-1) can be obtained by the methods described in J. Org. Chem. 2003, 68, 8715-8718; Journal of the Chemical Society; and Perkin Transactions 1: Organic and Bio-Organic Chemistry (1997), (22), 3471-3478.

(Composition)

The composition according to the present embodiment contains at least one selected from the group consisting of the polymer compound, a hole transport material, an electron transport material, and a light-emitting material. The composition can be suitably used in production of the light-emitting device, in the light-emitting device to be obtained, the light emission efficiency is excellent.

Examples of the hole transport material include polyvinylcarbazole and derivatives thereof, polysilane and derivatives thereof, polysiloxane derivatives having an aromatic amine in the side chain or the main chain, pyrazoline derivatives, arylamine derivatives, stilbene derivatives, polyaniline and derivatives thereof, polythiophene and derivatives thereof, polypyrrole and derivatives thereof, poly(p-phenylenevinylene) and derivatives thereof, and poly(2,5-thienylenevinylene) and derivatives thereof. Besides, examples thereof include the hole transport materials described in Japanese Patent Application Laid-Open Nos. 63-70257, 63-175860, 2-135359, 2-135361, 2-209988, 3-37992, and 3-152184.

The content of the hole transport material is preferably 1 to 500 part by weight, more preferably 5 to 200 part by weight based on 100 part by weight of the polymer compound in the composition.

Examples of the electron transport material include oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, metal complexes of 8-hydroxyquinoline and derivatives thereof, polyquinoline and derivatives thereof, polyquinoxaline and derivatives thereof, polyfluorene and derivatives thereof, anthracene and derivatives thereof, and copolymers of anthracene and fluorene. Besides, examples thereof include the electron transport materials described in Japanese Patent Application Laid-Open Nos. 63-70257, 63-175860, 2-135359, 2-135361, 2-209988, 3-37992, and 3-152184. The electron transport material may be a polymer compound having the constitutional unit represented by the formula (1) and not having the constitutional unit represented by the formula (2).

The content of the electron transport material is preferably 1 to 500 part by weight, more preferably 5 to 200 part by weight based on 100 part by weight of the polymer compound in the composition.

Examples of the light-emitting material include low molecular fluorescence light-emitting materials and phosphorescence light-emitting materials. Examples of the light-emitting material include naphthalene derivatives; anthracene and derivatives thereof; copolymers of anthracene and fluorene; perylene and derivatives thereof; dyes such as polymethine dyes, xanthene dyes, coumarin dyes, and cyanine dyes; metal complexes having 8-hydroxyquinoline as a ligand; metal complexes having 8-hydroxyquinoline derivatives as a ligand; other fluorescent metal complexes; aromatic amines; tetraphenylcyclopentadiene and derivatives thereof, tetraphenylbutadiene and derivatives thereof; low molecular compound fluorescent materials such as stilbene low molecular compounds, silicon-containing aromatic low molecular compounds, oxazole low molecular compounds, furoxan low molecular compounds, thiazole low molecular compounds, tetraarylmethane low molecular compounds, thiadiazole low molecular compounds, pyrazole low molecular compounds, metacyclophane low molecular compounds, and acetylene low molecular compounds; metal complexes such as iridium complexes and platinum complexes; and triplet light emitting complexes. Besides, examples thereof include the light-emitting materials described in Japanese Patent Application Laid-Open Nos. 57-51781, 59-194393, and others.

The content of the light-emitting material is preferably 1 to 500 part by weight, more preferably 5 to 200 part by weight based on 100 part by weight of the polymer compound in the composition.

(Liquid Composition)

The polymer compound according to the present embodiment may be dissolved or dispersed in a solvent, preferably in an organic solvent to prepare a liquid composition (solution or dispersion liquid). Such a liquid composition is also referred to as an ink or a varnish. In the case where the liquid composition is used to form an organic film used in the light-emitting device, it is preferable that the liquid composition be a solution.

In addition to the polymer compound according to the present embodiment, the liquid composition may contain at least one selected from the group consisting of the hole transport material, the electron transport material, and the light-emitting material (namely, one embodiment of the composition above). Moreover, other substance may be added to the liquid composition unless the effects of the present invention are prevented. Examples of the other substance include an antioxidant, a viscosity control agent, and a surfactant.

Here, the organic solvent is not particularly limited as long as the polymer compound according to the present embodiment is dissolved or dispersed; examples of the organic solvent include the following organic solvents (hereinafter, referred to as a “solvent groups” in some cases).

Aromatic hydrocarbon solvents: such as toluene, xylene (isomers or a mixture thereof), 1,2,3-trimethylbenzene, 1,2,4-trimethylbenzene, mesitylene (1,3,5-trimethylbenzene), ethylbenzene, propylbenzene, isopropylbenzene, butylbenzene, isobutylbenzene, 2-phenylbutane, tert-butylbenzene, pentylbenzene, neopentylbenzene, isoamylbenzene, hexylbenzene, cyclohexylbenzene, heptylbenzene, octylbenzene, 3-propyltoluene, 4-propyltoluene, 1-methyl-4-propylbenzene, 1,4-diethylbenzene, 1,4-dipropylbenzene, 1,4-di-tert-butylbenzene, indane, and tetralin (1,2,3,4-tetrahydronaphthalene).

Aliphatic hydrocarbon solvents: such as n-pentane, n-hexane, cyclohexane, methylcyclohexane, n-heptane, n-octane, n-nonane, n-decane, and decalin.

Aromatic ether solvents: such as anisole, ethoxybenzene, propoxybenzene, butyloxybenzene, pentyloxybenzene, cyclopentyloxybenzene, hexyloxybenzene, cyclohexyloxybenzene, heptyloxybenzene, octyloxybenzene, 2-methylanisole, 3-methylanisole, 4-methylanisole, 4-ethylanisole, 4-propylanisole, 4-butylanisole, 4-pentylanisole, 4-hexylanisole, diphenylether, 4-methylphenoxybenzene, 4-ethylphenoxybenzene, 4-propylphenoxybenzene, 4-butylphenoxybenzene, 4-pentylphenoxybenzene, 4-hexylphenoxybenzene, 4-phenoxytoluene, 3-phenoxytoluene, 1,3-dimethoxybenzene, 2,6-dimethylanisole, 2,5-dimethylanisole, 2,3-dimethylanisole, and 3,5-dimethylanisole.

Aliphatic ether solvents: such as tetrahydrofuran, dioxane, and dioxolane.

Ketone solvents: such as acetone, methyl ethyl ketone, methyl isobutyl ketone, cyclohexanone, and acetophenone.

Ester solvents: such as ethyl acetate, butyl acetate, methyl benzoate, and ethyl cellosolve acetate.

Chlorinated solvents: such as methylene chloride, chloroform, 1,2-dichloroethane, 1,1,2-trichloroethane, chlorobenzene, and o-dichlorobenzene.

Alcohol solvents: methanol, ethanol, propanol, isopropanol, cyclohexanol, and phenol.

Polyhydric alcohols and derivatives thereof: such as ethylene glycol, ethylene glycol monobutyl ether, ethylene glycol monoethyl ether, ethylene glycol monomethyl ether, dimethoxyethane, propylene glycol, diethoxymethane, triethylene glycol monoethyl ether, glycerol, and 1,2-hexanediol.

Aprotic polar solvents: such as dimethylsulfoxide, N-methyl-2-pyrrolidone, N,N-dimethylformamide, and N,N-dimethylacetoamide.

These organic solvents may be used alone, or two or more thereof may be used as a mixed solvent. In the case where the mixed solvent is used, it is preferable that two or three or more of the solvents in the solvent groups be used in combination; several solvents from the same solvent group described abovemay be used in combination, or one or more solvents from different solvent groups may be used in combination. The composition ratio can be determined considering the physical properties of the solvents, the solubility of the polymer compound, and the like.

Preferable examples in the case where several solvents are selected from the same solvent group and used in combination include several solvents from the aromatic hydrocarbon solvents, and several solvents from the aromatic ether solvents.

Preferable examples in the case where one or more solvents are selected from different solvent groups and used in combination include the following combinations:

the aromatic hydrocarbon solvent and the aliphatic hydrocarbon solvent;
the aromatic hydrocarbon solvent and the aromatic ether solvent;
the aromatic hydrocarbon solvent and the aliphatic ether solvents;
the aromatic hydrocarbon solvent and the aprotic polar solvent; and
the aromatic ether solvent and the aprotic polar solvent.
A single solvent or the mixed solvent can be added to water.

Among these organic solvents, a single solvent or mixed solvent containing one or more organic solvents having a structure including a benzene ring, a melting point of 0° C. or less, and a boiling point of 100° C. or more is preferable; among these, a single solvent or mixed solvent containing one or more of the aromatic hydrocarbon solvents and the aromatic ether solvents are particularly preferable from the viewpoint of viscosity and film forming properties.

These organic solvents can be used alone, or two or more thereof can be used in combination as a mixed solvent; from the viewpoint of the film forming properties, it is preferable that the mixed solvent be used. When necessary, the organic solvent may be refined by a method such as washing, distillation, and contacting with an adsorbent, and used.

According to the liquid composition, the organic film containing the polymer compound according to the present embodiment can be easily produced. Specifically, the liquid composition is applied onto a substrate, and the organic solvent is distilled away by heating, sending air, reducing pressure, or the like; thereby, the organic film containing the polymer compound according to the present embodiment is obtained. In the distillation of the organic solvent, the condition can be changed depending on the organic solvent to be used; examples of the condition include an atmosphere temperature of 50 to 150° C. (heating) or a reduced pressure atmosphere of approximately 10⁻³ Pa.

As the application, an application method such as a spin coating method, a casting method, a microgravure method, a gravure coating method, a bar coating method, a roll coating method, a wire bar coating method, a dip coating method, a slit coating method, a capillary coating method, a spray coating method, a screen printing method, a flexographic printing method, an offset printing method, an inkjet print method, and a nozzle coating method can be used.

A suitable viscosity of the liquid composition varies depending on the printing method; the viscosity at 25° C. is preferably 0.5 to 1000 mPa·s, and more preferably 0.5 to 500 mPa·s. In the case where the liquid composition is passed through an ejecting apparatus as in the inkjet print method, to prevent clogging and curved flight of ink droplets during ejection, the viscosity at 25° C. is preferably 0.5 to 50 mPa·s, and more preferably 0.5 to 20 mPa·s. The concentration of the polymer compound according to the present embodiment in the liquid composition is not particularly limited; it is preferable that the concentration be 0.01 to 10% by weight, and it is more preferable that the concentration be 0.1 to 5% by weight.

(Organic Film)

The organic film according to the present embodiment contains the polymer compound. The organic film according to the present embodiment can be easily produced from the liquid composition as above.

The organic film according to the present embodiment can be suitably used as a light-emitting layer in the light-emitting device described later. The organic film according to the present embodiment can also be suitably used for an organic semiconductor device. Because the organic film according to the present embodiment contains the polymer compound, the light emission efficiency of the light-emitting device is excellent in the case where the organic film is used as the light-emitting layer in the light-emitting device.

(Light-Emitting Device)

The light-emitting device according to the present embodiment has the organic film.

Specifically, the light-emitting device according to the present embodiment has an anode, a cathode, and a layer existing between the anode and the cathode and containing the polymer compound. Here, it is preferable that the layer containing the polymer compound be a layer formed of the organic film, and the layer function as the light-emitting layer. Hereinafter, the case where the layer containing the polymer compound functions as the light-emitting layer will be exemplified as preferable one embodiment.

Examples of the light-emitting device according to the present embodiment include light-emitting devices having the following structures (a) to (d). The symbol “/” designates that the layers before and after the symbol are adjacent and laminated (for example, “anode/light-emitting layer” designates that the anode and the light-emitting layer are adjacent and laminated).

(a) anode/light-emitting layer/cathode
(b) anode/hole transport layer/light-emitting layer/cathode
(c) anode/light-emitting layer/electron transport layer/cathode
(d) anode/hole transport layer/light-emitting layer/electron transport layer/cathode

The light-emitting layer is a layer having a light emission function; the hole transport layer is a layer having a function to transport holes; the electron transport layer is a layer having a function to transport electrons. The hole transport layer and the electron transport layer are collectively referred to as a charge transport layer in some cases. The hole transport layer adjacent to the light-emitting layer is referred to as an interlayer layer in some cases.

Lamination of the layers and film formation can be performed using a solution containing components that form each of the layers. In lamination and film forming from a solution, an application method such as a spin coating method, casting method, a microgravure coating method, a gravure coating method, a bar coating method, a roll coating method, a wire bar coating method, a dip coating method, a slit coating method, a capillary coating method, a spray coating method, a screen printing method, a flexographic printing method, an offset printing method, an inkjet print method, and a nozzle coating method can be used.

The thickness of the light-emitting layer may be selected such that the driving voltage and the light emission efficiency are proper values; the thickness is usually 1 nm to 1 preferably 2 nm to 500 nm, and still more preferably 5 nm to 200 nm.

It is preferable that the hole transport layer contain the hole transport material. Film formation of the hole transport layer may be performed by any method; in the case where the hole transport material is a polymer compound, it is preferable that film formation be performed from the solution containing the hole transport material; in the case where the hole transport material is a low-molecular compound, it is preferable that film formation be performed from a mixed liquid containing a polymer binder and the hole transport material. As the film forming method, the same method as the application method above can be used.

As the polymer binder that can be mixed with the hole transport material, a compound that does not extremely inhibit charge transportation and whose absorption of visible light is not strong is preferable. Examples of the polymer binder include polycarbonate, polyacrylate, polymethylacrylate, polymethylmethacrylate, polystyrene, polyvinyl chloride, and polysiloxane.

The thickness of the hole transport layer may be selected such that the driving voltage and the light emission efficiency are proper values; the thickness is usually 1 nm to 1 μm, preferably 2 nm to 500 nm, and still more preferably 5 nm to 200 nm.

It is preferable that the electron transport layer contain the electron transport material above. The film formation of the electron transport layer may be performed by any method; in the case where the electron transport material is a polymer compound, a method of forming a film from a solution containing the electron transport material, and a method of melting the electron transport material and forming a film are preferable. In the case where the electron transport material is a low-molecular compound, a method of forming a film using a powder of the electron transport material by a vacuum evaporation method, a method of forming a film from a solution containing the electron transport material, and a method of melting the electron transport material and forming a film are preferable. Examples of the method of forming a film from a solution containing the electron transport material can include the same method as the application method above. A polymer binder may be contained in the solution.

As the polymer binder that can be mixed with the electron transport material, a compound that does not extremely inhibit charge transportation and whose absorption of visible light is not strong is preferable. Examples of the polymer binder include poly(N-vinylcarbazole), polyaniline and derivatives thereof, polythiophene and derivatives thereof, poly(para-phenylenevinylene) and derivatives thereof, poly(2,5-thienylenevinylene) and derivatives thereof, polycarbonate, polyacrylate, polymethylacrylate, polymethyl methacrylate, polystyrene, polyvinyl chloride, and polysiloxane.

The thickness of the electron transport layer may be selected such that the driving voltage and the light emission efficiency are proper values; the thickness is usually 1 nm to 1 preferably 2 nm to 500 nm, and still more preferably 5 nm to 200 nm.

Among the charge transport layers provided adjacent to an electrode, a charge transport layer having a function to improve charge injection efficiency from the electrode and an effect of reducing the driving voltage of the device is particularly referred to as a charge injection layer (hole injection layer, electron injection layer) in some cases. In order to improve adhesion of the electrode and injection of charges from the electrode, the charge injection layer or insulating layer may be provided adjacent to the electrode; in order to improve adhesion of the interface and prevention of mixing, a thin buffer layer may be inserted into the interface between the charge transport layer and the light-emitting layer. The order and the number of the layers to be laminated and the thicknesses of the layers may be selected considering the light emission efficiency and luminance life.

Examples of the light-emitting device in which the charge injection layer is provided include light-emitting devices having the following structures (e) to (p):

(e) anode/charge injection layer/light-emitting layer/cathode
(f) anode/light-emitting layer/charge injection layer/cathode
(g) anode/charge injection layer/light-emitting layer/charge injection layer/cathode
(h) anode/charge injection layer/hole transport layer/light-emitting layer/cathode
(i) anode/hole transport layer/light-emitting layer/charge injection layer/cathode
(j) anode/charge injection layer/hole transport layer/light-emitting layer/charge injection layer/cathode
(k) anode/charge injection layer/light-emitting layer/charge transport layer/cathode
(l) anode/light-emitting layer/electron transport layer/charge injection layer/cathode
(m) anode/charge injection layer/light-emitting layer/electron transport layer/charge injection layer/cathode
(n) anode/charge injection layer/hole transport layer/light-emitting layer/charge transport layer/cathode
(o) anode/hole transport layer/light-emitting layer/electron transport layer/charge injection layer/cathode
(p) anode/charge injection layer/hole transport layer/light-emitting layer/electron transport layer/charge injection layer/cathode

Examples of the charge injection layer include (I) a layer containing a conductive polymer, (II) a layer provided between the anode and the hole transport layer and containing a material having an ionization potential of a middle value between the anode material in the anode and the hole transport material in the hole transport layer, and (III) a layer provided between the cathode and the electron transport layer and a layer containing a material having an electron affinity force of a middle value between the cathode material in the cathode and the electron transport material in the electron transport layer.

In the case where the charge injection layer is (I) the layer containing a conductive polymer, it is preferable that the electric conductivity of the conductive polymer be 10⁻⁵ S/cm to 10³ S/cm; in order to reduce the leak current between light-emitting pixels, it is more preferable that the electric conductivity of the conductive polymer be 10⁻⁵ S/cm to 10² S/cm, and it is still more preferable that the electric conductivity of the conductive polymer be 10⁻⁵ S/cm to 10¹ S/cm. In order to satisfy the range, the conductive polymer may be doped with a proper amount of ion.

The kind of ions to be doped with is an anion for a hole injection layer, and a cation for the electron injection layer. Examples of the anion include polystyrenesulfonic acid ion, alkylbenzenesulfonic acid ion, and camphorsulfonic acid ion. Examples of the cation include lithium ion, sodium ion, potassium ion, and tetrabutylammonium ion.

It is preferable that the thickness of the charge injection layer be 1 to 100 nm, and it is more preferable that the thickness of the charge injection layer be 2 to 50 nm.

The conductive polymer may be selected according to the relationship with the electrode and the material of the adjacent layer; examples thereof include conductive polymers such as polyaniline and derivatives thereof, polythiophene and derivatives thereof, polypyrrole and derivatives thereof, polyphenylenevinylene and derivatives thereof, polythienylenevinylene and derivatives thereof, polyquinoline and derivatives thereof, polyquinoxaline and derivatives thereof, and polymers including an aromatic amine structure in the main chain or side chain. Examples of the charge injection layer include metal phthalocyanines (such as copper phthalocyanine) and layers containing carbon or the like.

The insulating layer is a layer having a function to facilitate injection of charges. The thickness of the insulating layer is usually 0.1 to 20 nm, preferably 0.5 to 10 nm, and more preferably 1 to 5 nm. Examples of a material used as the insulating layer include metal fluorides, metal oxides, and organic insulating materials.

Examples of the light-emitting device in which the insulating layer is provided include light-emitting devices having the following structures (q) to (ab):

(q) anode/insulating layer/light-emitting layer/cathode
(r) anode/light-emitting layer/insulating layer/cathode
(s) anode/insulating layer/light-emitting layer/insulating layer/cathode
(t) anode/insulating layer/hole transport layer/light-emitting layer/cathode
(u) anode/hole transport layer/light-emitting layer/insulating layer/cathode
(v) anode/insulating layer/hole transport layer/light-emitting layer/insulating layer/cathode
(w) anode/insulating layer/light-emitting layer/electron transport layer/cathode
(x) anode/light-emitting layer/electron transport layer/insulating layer/cathode
(y) anode/insulating layer/light-emitting layer/electron transport layer/insulating layer/cathode
(z) anode/insulating layer/hole transport layer/light-emitting layer/electron transport layer/cathode
(aa) anode/hole transport layer/light-emitting layer/electron transport layer/insulating layer/cathode
(ab) anode/insulating layer/hole transport layer/light-emitting layer/electron transport layer/insulating layer/cathode

It is preferable that the light-emitting device according to the present embodiment have a substrate adjacent to the anode or the cathode. As the substrate, a substrate whose shape and properties do not change when the electrode and the layers are formed are preferable; examples thereof include substrates made of glass, plastics, polymer films, silicon, and the like. In the case of the non-transparent substrate, it is preferable that an electrode opposite to an electrode that the substrate contacts be transparent or semi-transparent.

In the light-emitting device according to the present embodiment, usually, at least one of the electrodes composed of the anode and the cathode is transparent or semi-transparent; it is preferable that the anode be transparent or semi-transparent.

As the material for the anode, conductive metal oxide films, semi-transparent metal films, and the like are used. Specifically, films produced using a conductive inorganic compound such as composite oxides formed of indium oxide, zinc oxide, tin oxide, and indium tin oxide (ITO) and composite oxides formed of indium zinc oxide, NESA, gold, platinum, silver, copper, and the like are used. As the anode, an organic transparent conductive film formed of polyaniline and derivatives thereof, polythiophene and derivatives thereof, and the like may be used. In order to facilitate injection of charges, a layer formed of a phthalocyanine derivative, a conductive polymer, carbon, or the like, or a layer formed of a metal oxide, a metal fluoride, an organic insulating material, or the like may be provided on the anode.

Examples of the method of producing the anode include a vacuum evaporation method, a sputtering method, an ion plating method, and a plating method.

The thickness of the anode can be selected considering light transmittance and electric conductivity; the thickness is usually 10 nm to 10 μm, preferably 20 nm to 1 μM, and still more preferably 30 nm to 500 nm.

As the material for the cathode, a material whose work function is small is preferable; a metal such as lithium, sodium, potassium, rubidium, cesium, beryllium, magnesium, calcium, strontium, barium, aluminum, scandium, vanadium, zinc, yttrium, indium, cerium, samarium, europium, terbium, and ytterbium, an alloy containing two or more of the metals, an alloy containing one or more of the metals and one or more of gold, silver, platinum, copper, manganese, titanium, cobalt, nickel, tungsten, and tin, graphite or a graphite interlayer compound, and the like are used.

As the method of producing the cathode, a vacuum evaporation method, a sputtering method, a lamination method for thermally pressing a metal film, and the like are used.

The thickness of the cathode can be selected considering electric conductivity and durability; the thickness is usually 10 nm to 10 μm, preferably 20 nm to 1 and still more preferably 50 nm to 500 nm.

A layer formed of a conductive polymer or a layer formed of a metal oxide, a metal fluoride, an organic insulating material, or the like may be provided between the cathode and the light-emitting layer or between the cathode and the electron transport layer; after production of the cathode, a protective layer for protecting the light-emitting device may be attached. In order to use the light-emitting device stably for a long time, it is preferable that a protective layer and/or a protective cover be attached to protect the light-emitting device from the outside.

As the protective layer, resins, metal oxides, metal fluorides, metal borides, and the like can be used. As the protective cover, a glass plate, a plastic plate whose surface is subjected to a low moisture permeation treatment; a method of bonding the protective cover to a device substrate with a thermosetting resin or a photocurable resin is suitably used. If a space is kept using a spacer, the device can be easily prevented from being scratched. If an inert gas such as nitrogen and argon is sealed in the space, oxidation of the cathode can be prevented; further, by providing a desiccant such as barium oxide inside of the space, suppression in moisture adsorbed during the production step damaging the device is easy.

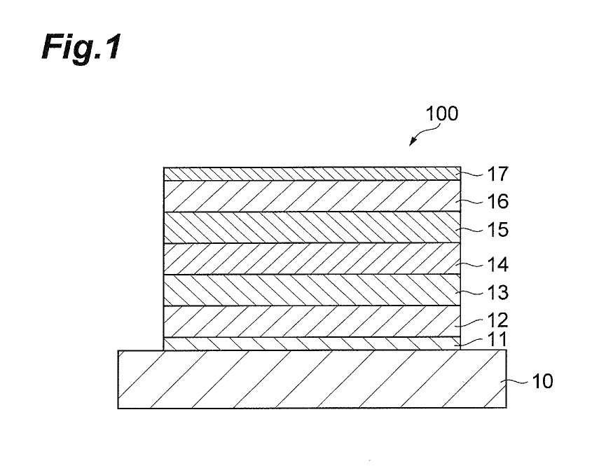

FIG. 1 is a schematic sectional view showing one embodiment of a light-emitting device according to the present invention (light-emitting device having the structure (p)). The light-emitting device 100 shown in FIG. 1 has a substrate 10, an anode 11 formed on the substrate 10, a hole injection layer 12, a hole transport layer 13, a light-emitting layer 14, an electron transport layer 15, an electron injection layer 16, and a cathode 17. The anode 11 is provided on the substrate 10 so as to contact the substrate 10; on a side of the anode 11 opposite to the substrate 10, the hole injection layer 12, the hole transport layer 13, the light-emitting layer 14, the electron transport layer 15, the electron injection layer 16, and the cathode 17 are laminated in this order.

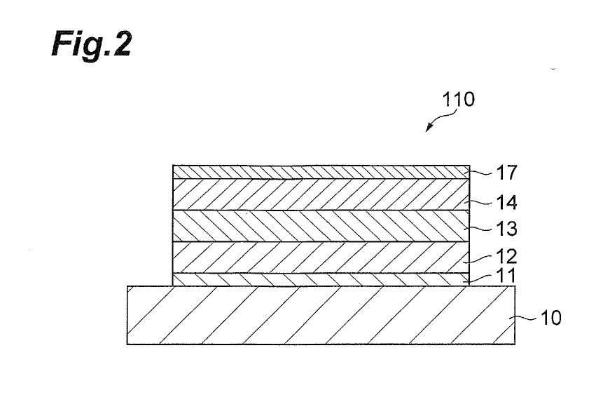

FIG. 2 is a schematic sectional view showing another embodiment of the light-emitting device according to the present invention (light-emitting device having the structure (h)). The light-emitting device 110 shown in FIG. 2 has a substrate 10, an anode 11 formed on the substrate 10, a hole injection layer 12, a hole transport layer 13, a light-emitting layer 14, and a cathode 17. The anode 11 is provided on the substrate 10 so as to contact the substrate; on a side of the anode 11 opposite to the substrate 10, the hole injection layer 12, the hole transport layer 13, the light-emitting layer 14, and the cathode 17 are laminated in this order.

The light-emitting device containing the polymer compound according to the present embodiment is useful for surface light sources such as curved surface light sources and flat surface light sources (such as lighting); and display devices such as segment display devices, dot matrix display devices (such as dot matrix flat displays), and liquid crystal display devices (for example, liquid crystal display devices and backlights of liquid crystal displays), for example. The polymer compound according to the present embodiment is suitable for the material used in production of these; besides, the polymer compound according to the present embodiment is also suitable for dyes for a laser, a material for a conductive film such as materials for an organic solar cell, organic semiconductors for an organic transistor, conductive films, organic semiconductor films, a light-emittable film material that emits fluorescence, a material for polymer field-effect transistors, and the like.

In order to obtain a planar light emission using the light-emitting device according to the present embodiment, a planar anode and cathode may be disposed so as to be layered. In order to obtain a patterned light emission, a method in which a mask in which a patterned window is provided is provided on the surface of the planar light-emitting device, and a method in which one of the anode and the cathode or both of the electrode are formed to be patterned are used. A pattern is formed by any of these methods, and some of electrodes are arranged to be capable of being turned ON/OFF independently; thereby, a segment display device on which numerals, letters, simple symbols, and the like can be displayed is obtained.

To obtain a dot matrix display device, the anode and the cathode both may be formed in a strip form and arranged intersecting perpendicular to each other. Partial color display and multicolor display are enabled by a method for applying polymer compounds of a plurality of different light-emitting colors, or a method using a color filter or a fluorescence conversion filter. The dot matrix display device can be passively driven, or may be actively driven in combination with a TFT or the like. These display devices can be used as display devices for computers, televisions, mobile terminals, mobile phones, car navigation systems, view finders for video cameras, and the like.

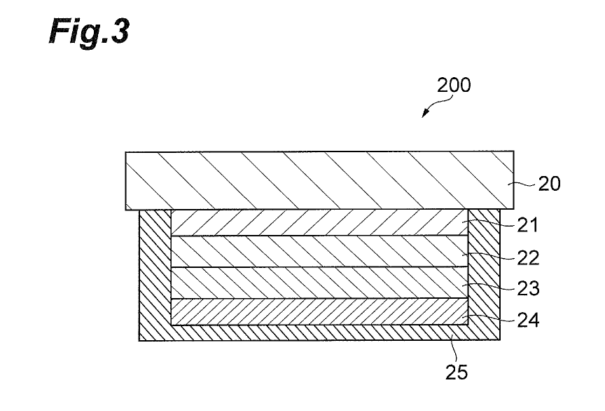

FIG. 3 is a schematic sectional view showing one embodiment of the surface light source according to the present invention. The surface light source 200 shown in FIG. 3 includes a substrate 20, an anode 21, a hole injection layer 22, a light-emitting layer 23, a cathode 24, and a protective layer 25. The anode 21 is provided on the substrate 20 so as to contact the substrate 20; on a side of the anode 21 opposite to the substrate 20, the hole injection layer 22, the light-emitting layer 23, and the cathode 24 are laminated in this order. The protective layer 25 is formed so as to cover all the anode 21, the charge injection layer 22, the light-emitting layer 23, and the cathode 24 formed on the substrate 20 and contact the substrate 20 at the end. The polymer compound is contained in the light-emitting layer 23.

The surface light source 200 shown in FIG. 3 is configured to further have a plurality of light-emitting layers other than the light-emitting layer 23, and can be formed as a color display device by using a red light-emitting material, a blue light-emitting material, and a green light-emitting material for each of the light-emitting layers and controlling drive of the light-emitting layers.

### EXAMPLES

Hereinafter, the present invention will be more specifically described using Examples, but the present invention will not be limited to Examples.

The polystyrene-equivalent number-average molecular weight and weight-average molecular weight of the polymer compound were determined using a gel permeation chromatograph (GPC) (made by SHIMADZU Corporation, trade name: LC-10Avp) on the following measurement condition.

<Measurement Condition>

The polymer compound to be measured was dissolved in tetrahydrofuran such that the concentration was approximately 0.05% by weight, and 10 μL of the solution was injected to the GPC. Tetrahydrofuran was used as a mobile phase for the GPC, and flowed at a flow rate of 2.0 mL/min. As a column, a PLgel MIXED-B (made by Polymer Laboratories Ltd.) was used. As a detector, a differential refractive index detector (made by SHIMADZU Corporation, trade name: RID-10A) was used.

Measurement of NMR was performed by dissolving 5 to 20 mg of a measurement sample in approximately 0.5 mL of an organic solvent and using an NMR (made by Varian, Inc., trade name: INOVA300).

Measurement of LC-MS was performed by the following method. A measurement sample was dissolved in a proper organic solvent (such as chloroform, tetrahydrofuran, ethyl acetate, and toluene) such that the concentration was 1 to 10 mg/mL, measured with an LC-MS (made by Agilent Technologies, Inc., trade name: 1100LCMSD), and analyzed. As a mobile phase for the LC-MS, ion exchange water, acetonitrile, tetrahydrofuran, or a mixed liquid thereof was used, and when necessary acetic acid was added. As a column, an L-column 2 ODS (3 μm) (made by Chemicals Evaluation and Research Institute, Japan, inner diameter: 4.6 mm, length: 250 mm, particle diameter: 3 μm) was used.

### Example 1

### Synthesis of Compound 4 and Compound 5>

### (Synthesis of Compound 2)

First, using Compound 1, Compound 2 was synthesized as follows.

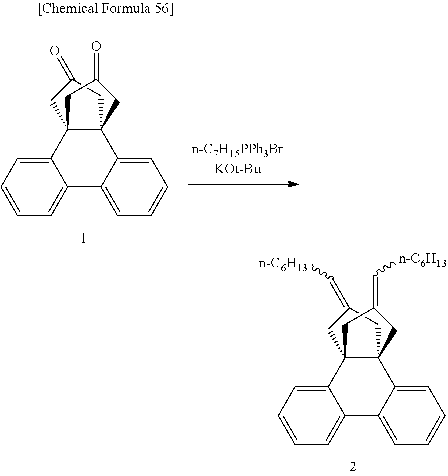

(wherein the wavy line indicates that the compound having the wavy line is a geometric isomer mixture).

Heptyltriphenylphosphonium bromide (115.0 g) was placed in a 1 L four-necked flask including a stirrer, and the gas inside of the flask was replaced with argon. Toluene (375 g) was put into the flask, and cooling was performed to 5° C. or less. Potassium tert-butoxide (29.2 g) was put into the flask, and the temperature was raised to room temperature; then, stirring was performed at room temperature for 3 hours while the temperature was kept. Compound 1 (15.0 g) was added to a red slurry produced during the reaction solution, and stirring was performed at room temperature for 12 hours while the temperature was kept. Acetic acid (10.0 g) was added to the reaction solution, and stirred for 15 minutes; then, the reaction solution was filtered to obtain a filtrate and a residue. Next, the residue was washed with toluene several times to obtain a washing liquid. Here, the filtrate and the washing liquid obtained by washing several times were mixed and condensed, and hexane was added thereto; then, a slurry was produced. The slurry was stirred at 50° C. for 1 hour while the temperature was kept. The obtained mixture was cooled to room temperature, and filtered to obtain a filtrate and a residue. Next, the residue was washed with hexane several times to obtain a washing liquid. Here, the filtrate and the washing liquid obtained by washing several times were mixed and condensed to obtain a crude product. The crude product was refined using a silica gel column (developing solvent of hexane) to obtain 21.7 g of Compound 2 as a colorless transparent liquid.

LC-MS (ESI, positive, KCl added): [M+K]⁺491.

¹H-NMR (CDCl₃, 300 MHz) δ (ppm): 0.87 (6H, t), 1.20-1.36 (16H, m), 1.82-1.97 (4H, m), 2.57-2.81 (8H, m), 5.20 (2H, br), 7.23-7.32 (4H, m), 7.41-7.48 (2H, m), 7.87-7.90 (2H, m).

(Synthesis of Compound 3)

Next, using Compound 2, Compound 3 was synthesized as follows.

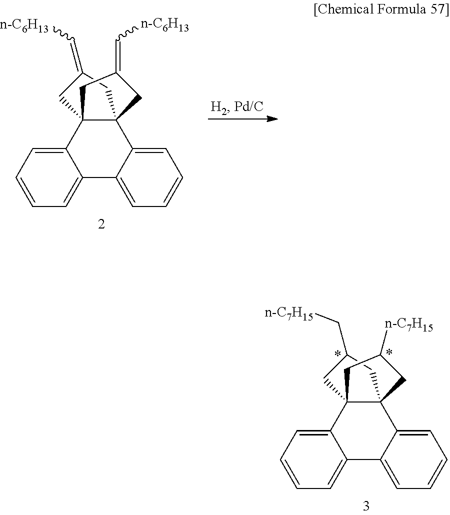

(wherein the wavy line indicates that the compound having the wavy line is a geometric isomer mixture; * indicates that a carbon atom to which * is attached is an asymmetric carbon atom).

Compound 2 (21.7 g) was placed in a 1 L four-necked flask including a stirrer; then, ethyl acetate (152.4 g) and ethanol (151.6 g) were placed in the flask, and the gas inside of the flask was replaced with nitrogen. 5% by weight Pd/C (a product containing approximately 50% by weight of water) (4.3 g) was added thereto; then, the gas inside of the flask was replaced with hydrogen; under a hydrogen atmosphere, stirring was performed at 40° C. for 27 hours while the temperature was kept. The obtained mixture was cooled to room temperature, and filtered with a filter precoated with celite to obtain a filtrate and a residue. Next, the residue was washed with ethyl acetate several times to obtain a washing liquid. Here, the filtrate and the washing liquid obtained by washing several times were mixed and condensed to obtain a crude product. The crude product was refined using a silica gel column (developing solvent of hexane) to obtain 21.7 g of Compound 3 as a colorless transparent liquid.

LC-MS (APPI, positive): [M]⁺456.

¹H-NMR (CDCl₃, 300 MHz) δ (ppm): 0.66-0.98 (6H, m), 1.00-2.22 (34H, m), 7.13-7.50 (6H, m), 7.80-7.98 (2H, m).

(Synthesis of Compound 4)

Next, using Compound 3, Compound 4 was synthesized as follows.

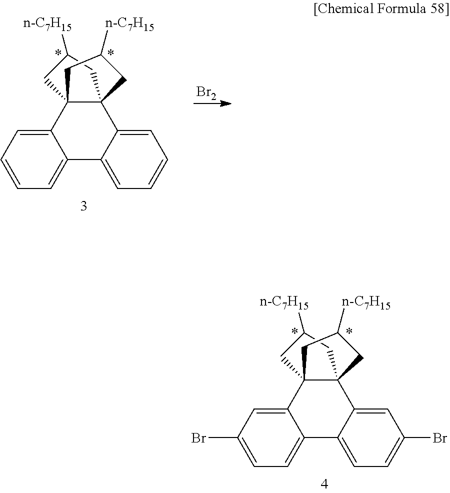

(wherein * indicates that a carbon atom to which * is attached is an asymmetric carbon atom).

Compound 3 (21.7 g), chloroform (261.1 g), and trifluoroacetic acid (44 g) were placed in a 500 mL four-necked flask including a stirrer, and the gas inside of the flask was replaced with argon. The entire four-necked flask was shielded from light, and a mixture of bromine (19.0 g) and chloroform (65.3 g) was dropped into the flask at room temperature over 15 minutes; then, the temperature was raised to 35° C. Stirring was performed at 35° C. for 7 hours while the temperature was kept; then, cooling was performed to 15° C. or less. A 10% by weight sodium sulfite aqueous solution (109 g) was added to the reaction solution, and the temperature was raised to room temperature. An aqueous layer was separated from the reaction solution, and an organic layer was washed with water, a 5% by weight sodium hydrogencarbonate aqueous solution, and water in this order. The obtained organic layer was dried with magnesium sulfate, and filtered; the filtrate was condensed to obtain a crude product. The crude product was recrystallized twice with a mixed liquid of ethanol and hexane. The obtained solid was dissolved in hexane, and refined using a silica gel column (developing solvent of hexane); activated carbon (2.1 g) was added to the obtained hexane solution, and stirring was performed at 45° C. for 1 hour while the temperature was kept. The obtained mixture was cooled to room temperature, and filtered with a filter precoated with celite; the residue was washed with hexane several times; the filtrates obtained by washing several times were added and partially condensed to obtain a hexane solution. Ethanol was added to the hexane solution to perform recrystallization; thereby, 18.8 g of Compound 4 was obtained as a white solid.

LC-MS (ESI, negative, KCl added): [M+Cl]⁻648.

¹H-NMR (CDCl₃, 300 MHz) δ (ppm): 0.66-0.98 (6H, m), 1.00-2.20 (34H, m), 7.22-7.78 (6H, m).

From the ¹H-NMR measurement result, it was found out that Compound 4 is a mixture of isomers with different stereochemistry (4a:4b:4c=51:39:10) (molar ratio).

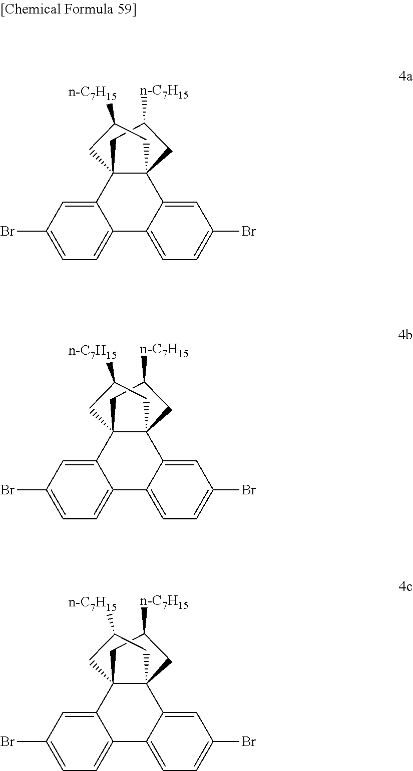

(Synthesis of Compound 5)

Next, using Compound 4, Compound 5 was synthesized as follows.

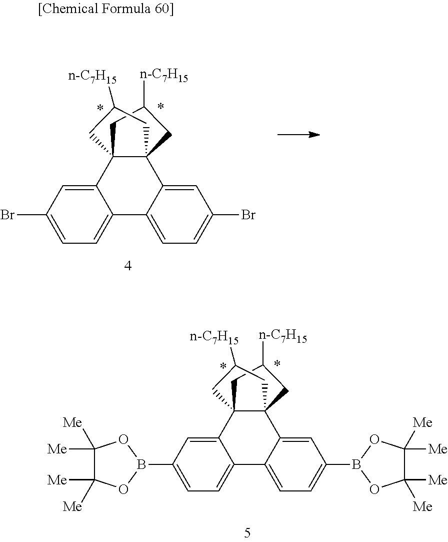

(wherein * indicates that a carbon atom to which * is attached is an asymmetric carbon atom).

Compound 4 (9.70 g), bispinacolatediboron (8.82 g), and potassium acetate (9.25 g) were placed in a 200 mL four-necked flask; then, the gas inside of the flask was replaced with nitrogen. 1,4-dioxane (95 mL), a palladium chloride (diphenylphosphinoferrocene)dichloromethane adduct (PdCl₂ (dppf) (CH₂Cl₂) (0.195 g), and diphenylphosphinoferrocene (dppf) (0.131 g) were added thereto, and stirred at 105° C. for 7 hours. The obtained solution was cooled to room temperature, and filtered a funnel precoated with celite. A condensed product obtained by condensing the filtrate by reducing pressure was dissolved in hexane, and activated carbon was added; stirring was performed while heating was performed at 40° C. for 1 hour. The obtained mixture was cooled to room temperature, and filtered with a funnel precoated with celite. A solid obtained by condensing the filtrate by reducing pressure was recrystallized with a mixed solvent of toluene and acetonitrile to obtain 9.0 g of Compound 5 as a white solid.

LC-MS (ESI, positive, KCl added): [M+K]⁺747.

### Example 2

### Synthesis of Compound 9

(Synthesis of 1-bromo-3,5,5-trimethylhexane)

Next, using 3,5,5-trimethylhexanol, 1-bromo-3,5,5-trimethylhexane was synthesized as follows.

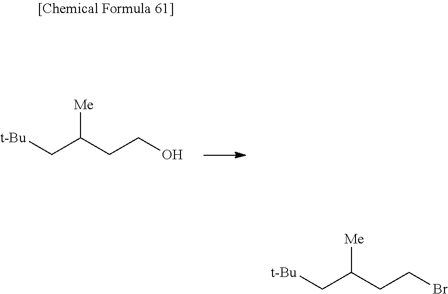 [SMILES: CC(CCO)CC(C)(C)C ; CC(CCBr)CC(C)(C)C]

3,5,5-trimethylhexanol (72.1 g) and triphenylphosphine (157.4 g) were placed in a 500 mL flask including a stirrer, and the gas inside of the flask was replaced with nitrogen. 121 mL of chloroform was placed in the flask; the flask was cooled with an ice bath; N-bromosuccinimide (106.8 g) was divided and dropped. The ice bath was removed, and the reaction solution was stirred at room temperature for 1 hour. A 10% by weight sodium carbonate aqueous solution (200 mL) was added; the aqueous layer was separated, and the organic layer was diluted with hexane. A precipitated solid was filtered, and condensed; then, the obtained condensed residue was refined with a silica gel column (developing solvent: hexane) to obtain 95.3 g of 1-bromo-3,5,5-trimethylhexane.

(Synthesis of Compound 6)

Next, using 1-bromo-3,5,5-trimethylhexane, Compound 6 was synthesized as follows.

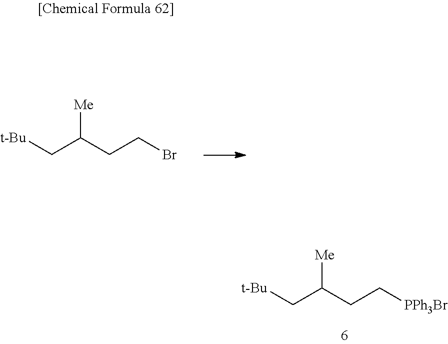

1-bromo-3,5,5-trimethylhexane (104.8 g), 120.6 g of triphenylphosphine, and toluene (139 mL) were placed in a 500 mL flask including a stirrer, and the gas inside of the flask was replaced with nitrogen. The temperature of the obtained mixture was raised to a reflux temperature, and refluxing was performed for 20 hours. The reaction solution was cooled to room temperature, and a precipitated solid was filtered. The obtained solid was washed with hexane three times while stirring was performed, and dried by reducing pressure to obtain 171.7 g of Compound 6.

(Synthesis of Compound 7)

Next, using Compound 6, Compound 7 was synthesized as follows.

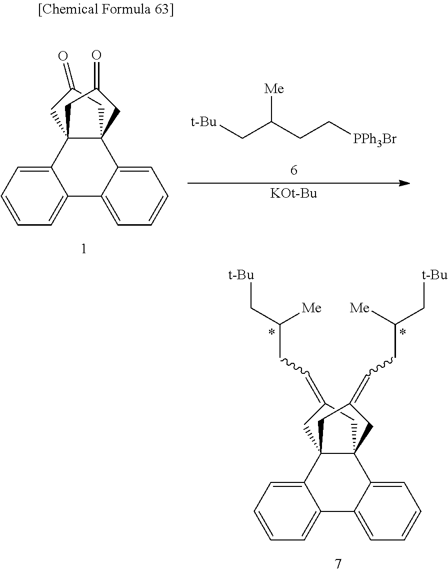

(wherein the wavy line indicates that the compound having the wavy line is a geometric isomer mixture; * indicates that a carbon atom to which * is attached is an asymmetric carbon atom).

Compound 6 (169 g) was placed in a 1 L four-necked flask including a stirrer, and the gas inside of the flask was replaced with nitrogen. Toluene (594 mL) was placed in the flask, and cooling was performed to 5° C. or less. Potassium tert-butoxide (39.2 g) was added, and the temperature was raised to room temperature; then, stirring was performed at room temperature for 3 hours while the temperature was kept. Compound 1 (20.1 g) was added to the red slurry produced during the reaction solution, and stirring was performed at room temperature for 20 hours while the temperature was kept. Acetic acid (13 mL) was added to the reaction solution, stirred for 15 minutes, and filtered to obtain a filtrate and a residue. Next, the residue was washed with toluene several times to obtain a washing liquid. The filtrate and the washing liquid obtained by washing several times were mixed and condensed, and hexane was added; then, a slurry was produced. The slurry was stirred at 50° C. for 1 hour while the temperature was kept. The obtained mixture was cooled to room temperature, and filtered to obtain a filtrate and a residue. Next, the residue was washed with hexane several times to obtain a washing liquid. The filtrate and the washing liquid obtained by washing several times were mixed and condensed; thereby, a crude product was obtained. The crude product was refined using a silica gel column (developing solvent of hexane) to obtain 34.5 g of Compound 7 as a colorless transparent liquid.

(Synthesis of Compound 8)

Next, using Compound 7, Compound 8 was synthesized as follows.

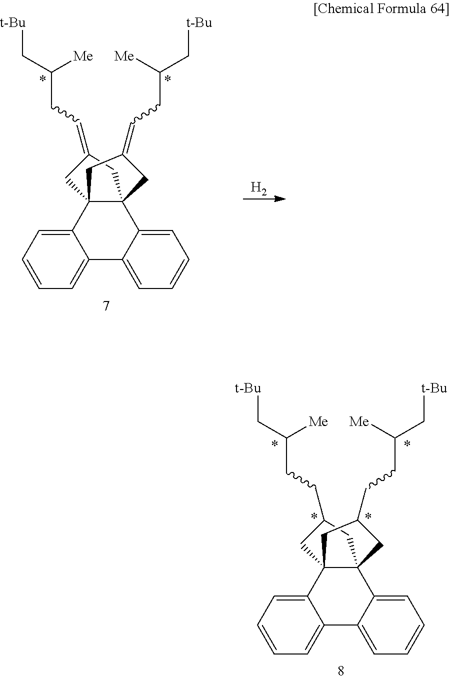

(wherein the wavy line indicates that the compound having the wavy line is a geometric isomer mixture; * indicates that a carbon atom to which * is attached is an asymmetric carbon atom).

Compound 7 (35.8 g) was placed in a 1 L four-necked flask including a stirrer; then, ethyl acetate (278 mL) and ethanol (318 mL) were placed in the flask, and the gas inside of the flask was replaced with nitrogen. 5% by weight Pd/C (a product containing approximately 50% by weight of water) (7.2 g) was placed in the flask; then, the gas inside of the flask was replaced with hydrogen; under a hydrogen atmosphere, stirring was performed at 40° C. for 30 hours while the temperature was kept. The obtained mixture was cooled to room temperature, and filtered with a filter precoated with celite to obtain a filtrate and a residue. Next, the residue was washed with ethyl acetate several times to obtain a washing liquid. The filtrate and the washing liquid obtained by washing several times were mixed and condensed to obtain a crude product. The crude product was refined using a silica gel column (developing solvent of hexane) to obtain 34.0 g of Compound 8 as a colorless transparent liquid.

LC-MS (ESI, negative, KCl added): [M-FC1]^{˜}547.

(Synthesis of Compound 9)

Next, using Compound 8, Compound 9 was synthesized as follows.

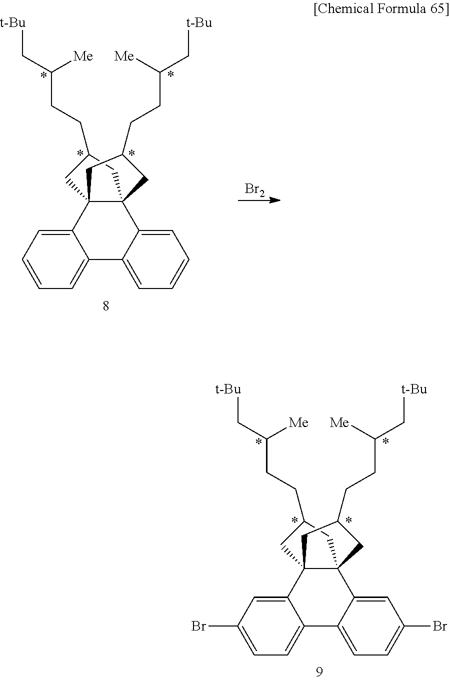

(wherein * indicates that a carbon atom to which * is attached is an asymmetric carbon atom).

Compound 8 (31.5 g), chloroform (298 mL), and trifluoroacetic acid (63 g) were placed in a 500 mL four-necked flask including a stirrer, and the gas inside of the flask was replaced with nitrogen. The entire four-necked flask was shielded from light; a mixture of bromine (24.5 g) and chloroform (21 mL) was dropped into the flask at room temperature over 15 minutes; then, the temperature was raised to 30° C. The obtained mixture was stirred at 30° C. for 7 hours while the temperature was kept; then, cooling was performed to 15° C. or less. A 10% by weight sodium sulfite aqueous solution (46 mL) was added to the reaction solution, and the temperature was raised to room temperature. An aqueous layer was separated from the reaction solution, and an organic layer was washed with water, a 5% by weight sodium hydrogencarbonate aqueous solution, and water in this order. The obtained organic layer was dried with magnesium sulfate, and filtered; the filtrate was condensed to obtain a crude product. The crude product was recrystallized twice with a mixed liquid of ethanol and hexane. The obtained solid was dissolved in hexane, and refined using a silica gel column (developing solvent of hexane); activated carbon (2.1 g) was added to the obtained hexane solution, and stirring was performed at 45° C. for 1 hour while the temperature was kept. The obtained mixture was cooled to room temperature, and filtered with a filter precoated with celite to obtain a filtrate and a residue. Next, the residue was washed with hexane several times to obtain a washing liquid. The filtrate and the washing liquid obtained by washing several times were mixed, and partially condensed to obtain a hexane solution. Ethanol was added to the hexane solution to perform recrystallization; thereby, 24.7 g of Compound 9 was obtained as a white solid.

LC-MS (ESI, negative, KCl added): [M+Cl]⁻705.

¹H-NMR (CDCl₃, 300 MHz) δ (ppm): 0.75-1.36 (38H, m), 1.56-1.82 (5H, m) 2.17-2.24 (5H, m), 7.33-7.68 (6H, m).

### Synthesis Example 1

### Synthesis of Compound 12

### (Synthesis of Compound 10)

Next, using 1,5-naphthyl bis(trifluoromethanesulfonate), Compound 10 was synthesized as follows.

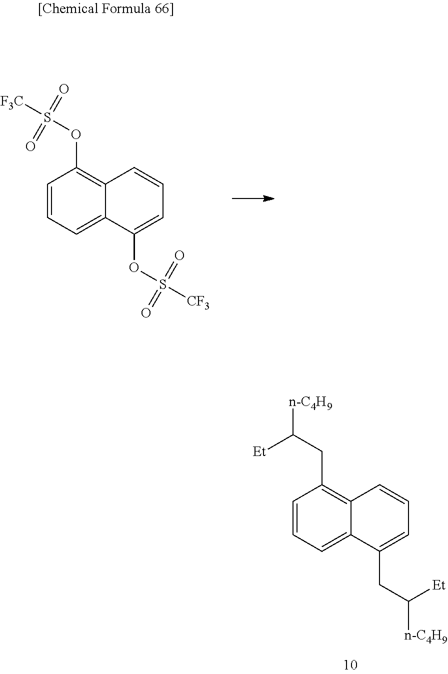

Under a nitrogen atmosphere, 1,5-naphthyl bis(trifluoromethanesulfonate) (25.0 g), a [1,1′-bis(diphenylphosphino)ferrocene]dichloropalladium (II)dichloromethylene adduct (0.24 g), and tert-butylmethylether (410 mL) were prepared, 2-ethylhexylmagnesium bromide (173 mL of a 1 mol/L diethyl ether solution) was dropped at 10° C. or less, and stirring was performed at room temperature for 4 hours. After the reaction was completed, the reaction solution was poured to a mixed liquid of water (500 ml) and 2 N hydrochloric acid (100 ml), and extracted with ethyl acetate; the obtained organic layer was washed with a sodium chloride aqueous solution; the washed organic layer was dried with magnesium sulfate, and the solvent was distilled away under reduced pressure. The residue was refined by silica gel column chromatography (developing solvent of hexane) to obtain 21.3 g of Compound 10 as a light yellow oil product.

LC-MS (ESI, positive): [M⁺]353.

¹H-NMR (300 MHz, CDCl₃) δ (ppm) 0:.75-1.00 (12H, m), 1.10-1.50 (16H, m), 1.69-1.85 (2H, m), 2.90-3.05 (4H, m), 7.24-7.38 (3H, m), 7.35-7.44 (3H, m), 7.90-7.95 (3H, m).

(Synthesis of Compound 11)

Next, using Compound 10, Compound 11 was synthesized as follows.

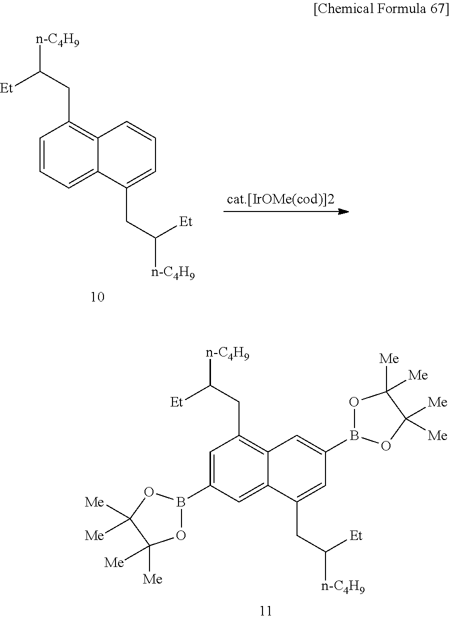

Under a nitrogen atmosphere, a mixture of Compound 10 (21.3 g), bis(pinacolate)diboron (4,4,4%4%5,5,5′,5′-octamethyl-2,2′-bi-1,3,2-dioxaborolane) (46.0 g), bis(1,5-cyclooctadiene)di-μ-methoxydiiridium(I) (0.24 g) (made by Sigma-Aldrich Corporation), 4,4′-ditert-butyl-2,2′-dipyridyl (0.19 g), and dioxane (140 mL) was stirred at 100° C. for 3 hours. The obtained mixture was cooled, and dioxane was distilled away under reduced pressure. Methanol (200 mL) was added to the residue, and a precipitated solid was filtered out, and dried. The obtained solid was dissolved in toluene (250 mL), and activated clay (20 g) was added; the solution was stirred at 60° C. for 30 minutes, and filtered with a filter precoated with silica gel while the solution was hot; the obtained filtrate was condensed under reduced pressure. Methanol (250 mL) was added to the obtained condensed product; a precipitated solid was filtered out, and dried to obtain 28.0 g of Compound 11 as a white powder.

LC-MS (ESI, positive): [M⁴]605.

¹H-NMR (300 MHz, CDCl₃) δ (ppm): 0.85-0.95 (12H, m), 1.24-1.50 (16H, m), 1.66-1.85 (2H, m), 2.90-3.18 (4H, m), 7.60 (2H, s), 8.47 (2H, s).

(Synthesis of Compound 12)

Next, using Compound 11, Compound 12 was synthesized as follows.

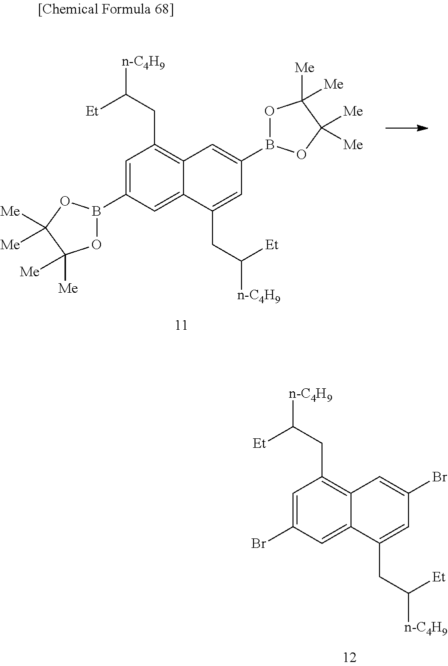

Under a nitrogen atmosphere, copper bromide(II) (62.7 g) was added to a mixed liquid of Compound 11 (28.0 g), dioxane (420 mL), N,N-dimethylformamide (420 mL), and water (210 mL), and stirring was performed at 95° C. for 2 hours. Further, copper bromide(II) (31.4 g) was added at the same temperature, and stirring was performed for 1.5 hours. Then, copper bromide(II) (31.4 g) was further added at the same temperature, and stirring was performed for 1.5 hours. The reaction solution was cooled; hexane (300 mL) was added, and stirring was performed. Then, an organic layer was separated, and dried with magnesium sulfate; the solvent was distilled away under reduced pressure. The residue was refined by silica gel column chromatography (developing solvent of hexane), and condensed to obtain a solid (21.0 g). The obtained solid was dissolved in toluene (150 mL), activated carbon (5 g) was added, and stirring was performed at 60° C. for 30 minutes. Then, the obtained mixture was filtered with a filter precoated with celite while the mixture was hot, and the obtained filtrate was condensed under reduced pressure. The obtained condensed product was recrystallized with a mixed liquid of toluene and methanol to obtain 13.2 g of Compound 12 as a white solid.

LC-MS (ESI, positive) [M]⁺511.

¹H-NMR (300 MHz, CDCl₃) δ (ppm): 0.80-0.98 (12H, m), 1.20-1.44 (16H, m), 1.64-1.80 (2H, m), 2.77-2.95 (4H, m), 7.37 (2H, s), 8.00 (2H, s).

### Example 3

### Synthesis of Polymer Compound A1

Synthesis of a polymer (Polymer Compound A1) having the constitutional unit represented by the following formula (K-1), the constitutional unit represented by the following formula (K-2), the constitutional unit represented by the following formula (K-3), and the constitutional unit represented by the following formula (K-4) at a molar ratio of 20:50:25:5 (a theoretical value based on prepared raw materials) was performed as follows.

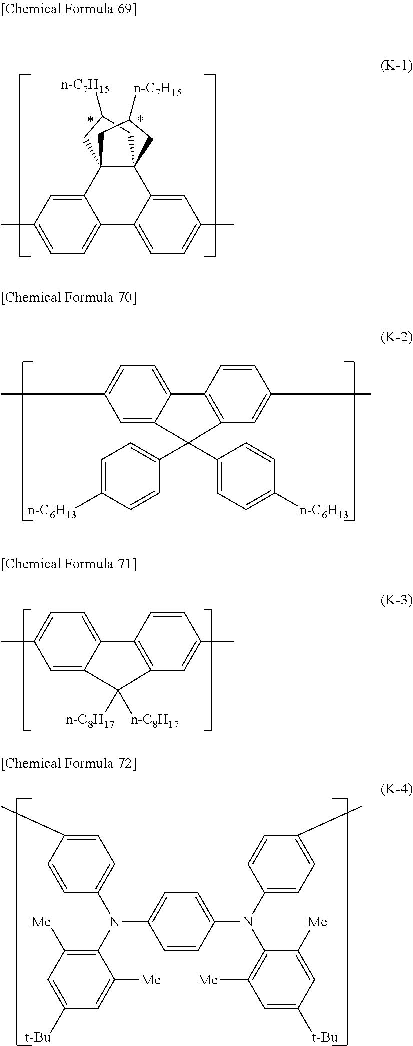

Under an argon atmosphere, Compound 4 synthesized in Example 1 (0.492 g, 0.80 mmol), the compound (1.477 g, 2.00 mmol) represented by the following formula (M-2-E):

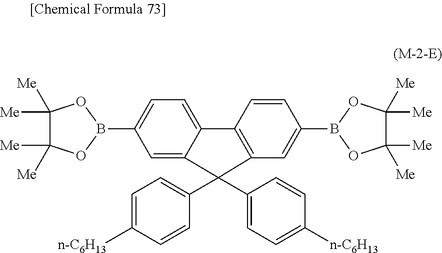

the compound (0.548 g, 1.00 mmol) represented by the following formula (M-3-BR):

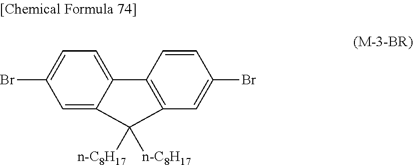

and the compound (0.148 g, 0.20 mmol) represented by the following formula (M-4-BR):

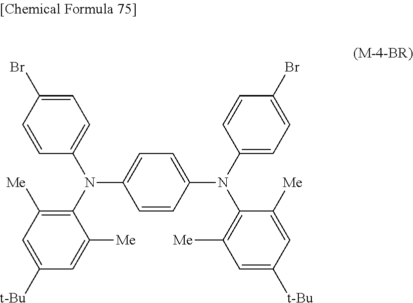 [SMILES: Cc1cc(C(C)(C)C)cc(C)c1N(c1ccc(Br)cc1)c1ccc(N(c2ccc(Br)cc2)c2c(C)cc(C(C)(C)C)cc2C)cc1]

dichlorobis(tris(o-methoxyphenyl))phosphinepalladium (1.77 mg), and toluene (50 mL) were mixed, and heated at 105° C.

A 20% by weight tetraethylammonium hydroxide aqueous solution (6.6 mL) was dropped into the reaction solution, and refluxing was performed for 2 hours 40 minutes. After the reaction, phenylboronic acid (24 mg) and toluene (5 mL) were added to the reaction solution, and refluxing was further performed for 18.5 hours. Next, a sodium diethyldithiocarbamate aqueous solution was added to the reaction solution, and stirring was performed at 80° C. for 2 hours. The obtained mixture was cooled, and an organic layer was washed twice with water, twice with a 3% by weight acetic acid aqueous solution, and twice with water. The obtained solution was dropped into methanol; then, a precipitate was produced, and filtered out to obtain a precipitate. The precipitate was dissolved in toluene, and the solution was passed through an alumina column and a silica gel column sequentially; thereby, the solution was refined. The obtained solution was dropped into methanol, and stirred; the obtained precipitate was filtered out, and dried to obtain 1.25 g of Polymer Compound A1. The polystyrene-equivalent number-average molecular weight of Polymer Compound A1 was 1.30×10⁵, and the polystyrene-equivalent weight-average molecular weight thereof was 3.26×10⁵.

### Example 4

### Synthesis of Polymer Compound A2

Synthesis of a polymer (Polymer Compound A2) having the constitutional unit represented by the following formula (K-5), the constitutional unit represented by the following formula (K-2), the constitutional unit represented by the following formula (K-6), and the constitutional unit represented by the following formula (K-4) at a molar ratio of 20:50:25:5 (a theoretical value based on prepared raw materials) was performed as follows.

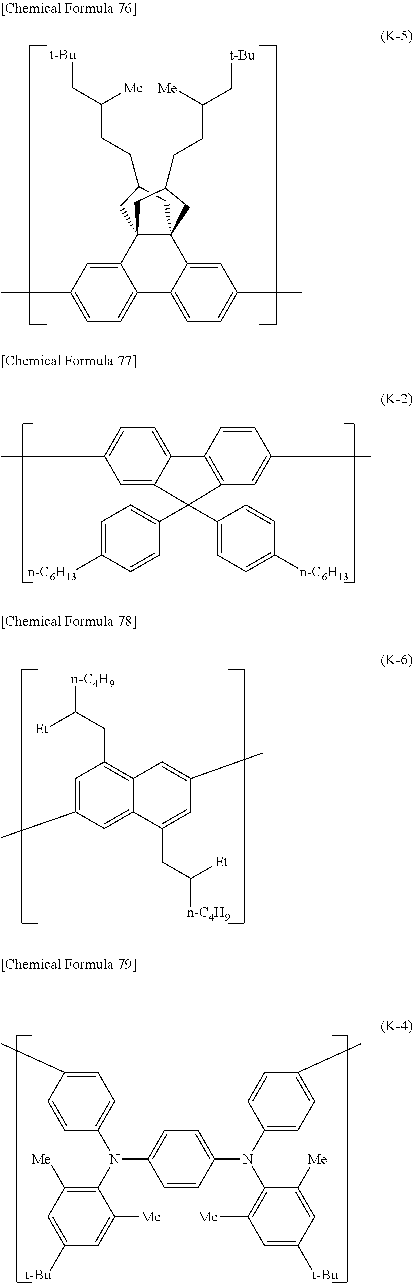

Under an argon atmosphere, Compound 9 synthesized in Example 2 (0.537 g, 0.80 mmol), the compound (1.477 g, 2.00 mmol) represented by the following formula (M-2-E):

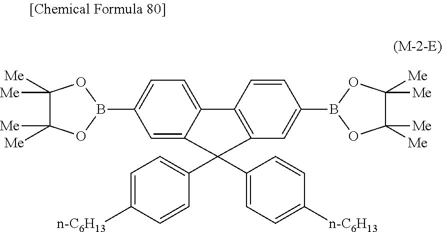

Compound 12 synthesized in Synthesis Example 1 (0.510 g, 1.00 mmol), the compound (0.148 g, 0.20 mmol) represented by the following formula (M-4-BR):

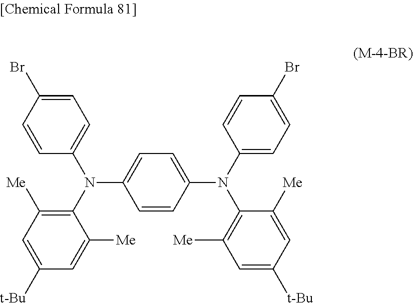 [SMILES: Cc1cc(C(C)(C)C)cc(C)c1N(c1ccc(Br)cc1)c1ccc(N(c2ccc(Br)cc2)c2c(C)cc(C(C)(C)C)cc2C)cc1]

dichlorobis(tris(o-methoxyphenyl))phosphinepalladium (1.77 mg), and toluene (50 mL) were mixed, and heated at 105° C.

A 20% by weight tetraethylammonium hydroxide aqueous solution (6.6 mL) was dropped into a reaction solution, and refluxing was performed for 3 hours. After the reaction, phenylboronic acid (24 mg) and toluene (5 mL) were added to the reaction solution, and refluxing was further performed for 18 hours. Next, a sodium diethyldithiocarbamate aqueous solution was added to the reaction solution, and stirring was performed at 80° C. for 2 hours. The obtained mixture was cooled, and an organic layer was washed twice with water, twice with a 3% by weight acetic acid aqueous solution, and twice with water. The obtained solution was dropped into methanol; then, a precipitate was produced, and filtered out to obtain a precipitate. The precipitate was dissolved in toluene, and the solution was passed through an alumina column and a silica gel column sequentially; thereby, the solution was refined. The obtained solution was dropped into methanol, and stirred; the obtained precipitate was filtered out, and dried to obtain 1.25 g of Polymer Compound A2. The polystyrene-equivalent number-average molecular weight of Polymer Compound A2 was 1.06×10⁵, and the polystyrene-equivalent weight-average molecular weight thereof was 2.53×10⁵.

### Example 5

### Synthesis of Polymer Compound A3

Synthesis of a polymer (Polymer Compound A3) having the constitutional unit represented by the following formula (K-1), the constitutional unit represented by the following formula (K-7), the constitutional unit represented by the following formula (K-4), and the constitutional unit represented by the following formula (K-8) at a molar ratio of 50:45:3:2 (a theoretical value based on prepared raw materials) was performed as follows.

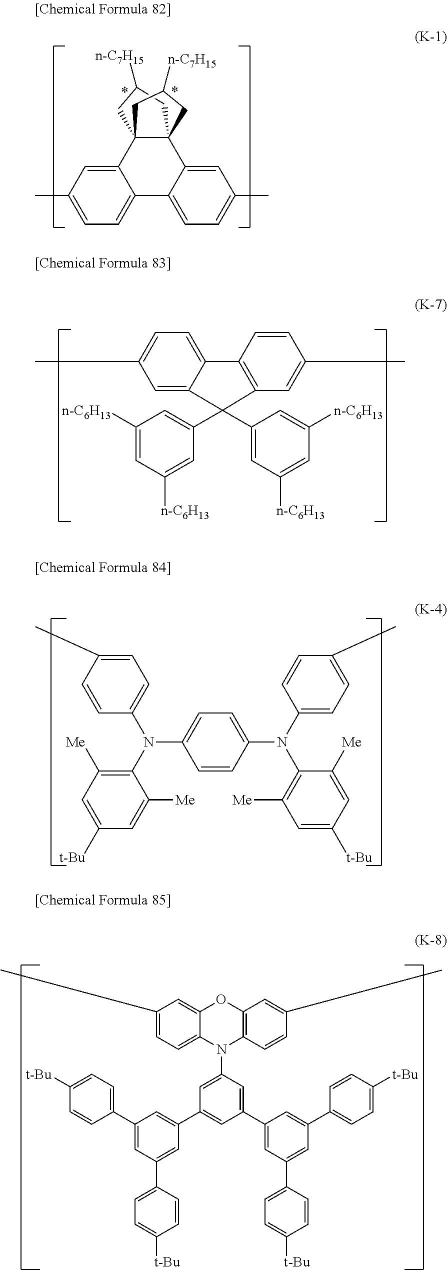

Under an argon atmosphere, Compound 5 synthesized in Example 1 (1.387 g, 2.00 mmol), the compound (1.463 g, 1.80 mmol) represented by the following formula (M-7-B):

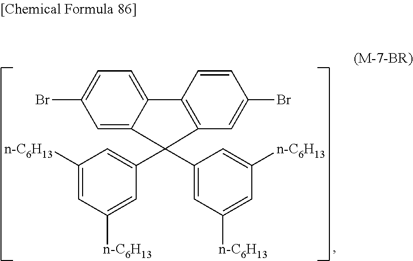

the compound (0.089 g, 0.12 mmol) represented by the following formula (M-4-BR):

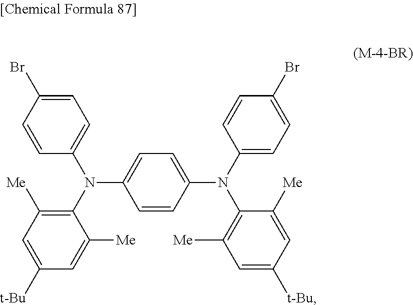 [SMILES: Cc1cc(C(C)(C)C)cc(C)c1N(c1ccc(Br)cc1)c1ccc(N(c2ccc(Br)cc2)c2c(C)cc(C(C)(C)C)cc2C)cc1]

the compound (0.088 g, 0.08 mmol) represented by the following formula (M-8-BR):

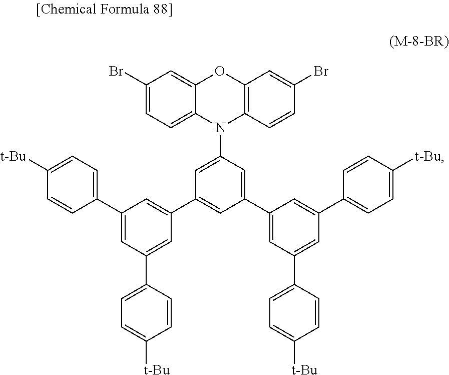 [SMILES: CC(C)(C)c1ccc(-c2cc(-c3ccc(C(C)(C)C)cc3)cc(-c3cc(-c4cc(-c5ccc(C(C)(C)C)cc5)cc(-c5ccc(C(C)(C)C)cc5)c4)cc(N4c5ccc(Br)cc5Oc5cc(Br)ccc54)c3)c2)cc1]

dichlorobis(tris(o-methoxyphenyl))phosphinepalladium (1.77 mg), and toluene (50 mL) were mixed, and heated at 105° C.

A 20% by weight tetraethylammonium hydroxide aqueous solution (6.6 mL) was dropped into a reaction solution, and refluxing was performed for 3 hours. After the reaction, phenylboronic acid (24 mg) and toluene (5 mL) were added to the reaction solution, and refluxing was further performed for 18 hours. Next, a sodium diethyldithiocarbamate aqueous solution was added to the reaction solution, and stirring was performed at 80° C. for 2 hours. The obtained mixture was cooled, and an organic layer was washed twice with water, twice with a 3% by weight acetic acid aqueous solution, and twice with water. The obtained solution was dropped into methanol; then, a precipitate was produced, and filtered out to obtain a precipitate. The precipitate was dissolved in toluene, and the solution was passed through an alumina column and a silica gel column sequentially; thereby, the solution was refined. The obtained solution was dropped into methanol, and stirred; the obtained precipitate was filtered out, and dried to obtain 1.19 g of Polymer Compound A3. The polystyrene-equivalent number-average molecular weight of Polymer Compound A3 was 2.04×10⁵, and the polystyrene-equivalent weight-average molecular weight thereof was 5.39×10⁵.

### Example 6

### Synthesis of Polymer Compound A4

Synthesis of a polymer (Polymer Compound A4) having the constitutional unit represented by the following formula (K-1), the constitutional unit represented by the following formula (K-2), the constitutional unit represented by the following formula (K-3), and the constitutional unit represented by the following formula (K-9) at a molar ratio of 50:25:20:5 (a theoretical value based on prepared raw materials) was performed as follows.

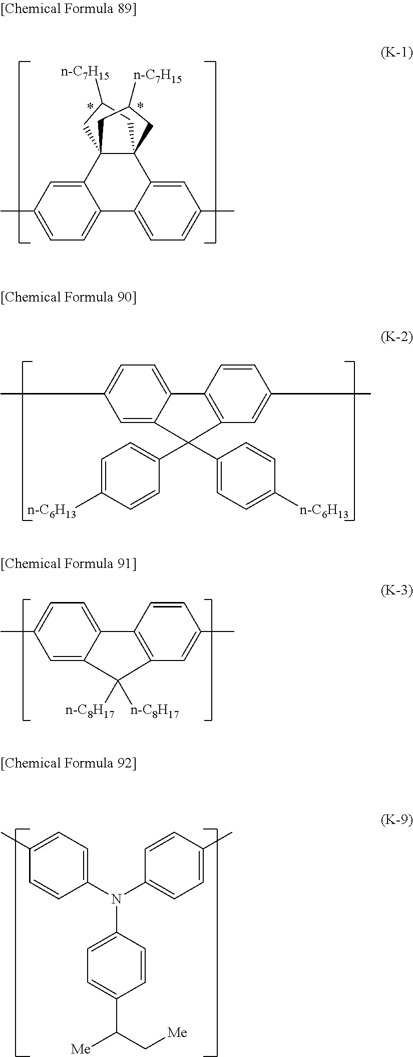

Under an argon atmosphere, Compound 4 synthesized in Example 1 (0.492 g, 0.80 mmol), the compound (1.477 g, 2.00 mmol) represented by the following formula (M-2-E):

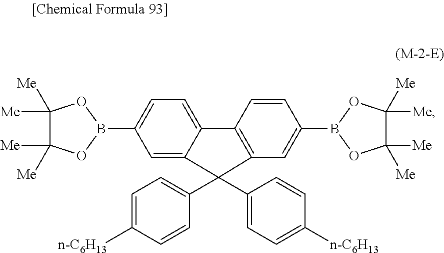

the compound (0.548 g, 1.00 mmol) represented by the following formula (M-3-BR):

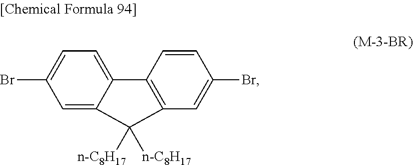

the compound (0.092 g, 0.20 mmol) represented by the following formula (M-9-BR):

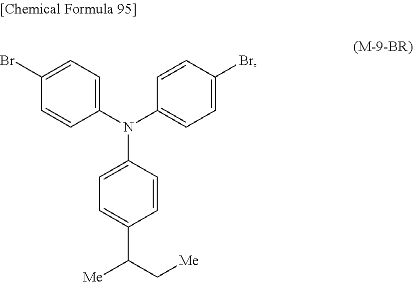 [SMILES: CCC(C)c1ccc(N(c2ccc(Br)cc2)c2ccc(Br)cc2)cc1]

dichlorobis(tris(o-methoxyphenyl))phosphinepalladium (1.77 mg), and toluene (50 mL) were mixed, and heated at 105° C.

A 20% by weight tetraethylammonium hydroxide aqueous solution (6.6 mL) was dropped into a reaction solution, and refluxing was performed for 3 hours. After the reaction, phenylboronic acid (24 mg) and toluene (5 mL) were added to the reaction solution, and refluxing was further performed for 18 hours. Next, a sodium diethyldithiocarbamate aqueous solution was added to the reaction solution, and stirring was performed at 80° C. for 2 hours. The obtained mixture was cooled, and an organic layer was washed twice with water, twice with a 3% by weight acetic acid aqueous solution, and twice with water. The obtained solution was dropped into methanol; then, a precipitate was produced, and filtered out to obtain a precipitate. The precipitate was dissolved in toluene, and the solution was passed through an alumina column and a silica gel column sequentially; thereby, the solution was refined. The obtained solution was dropped into methanol, and stirred; the obtained precipitate was filtered out, and dried to obtain 1.20 g of Polymer Compound A4. The polystyrene-equivalent number-average molecular weight of Polymer Compound A4 was 1.10×10⁵, and the polystyrene-equivalent weight-average molecular weight thereof was 2.89×10⁵.

### Example 7

### Synthesis of Polymer Compound A5

Synthesis of a polymer (Polymer Compound A5) having the constitutional unit represented by the following formula (K-1), the constitutional unit represented by the following formula (K-2), the constitutional unit represented by the following formula (K-10), and the constitutional unit represented by the following formula (K-4) at a molar ratio of 20:60:15:5 (a theoretical value based on prepared raw materials) was performed as follows.

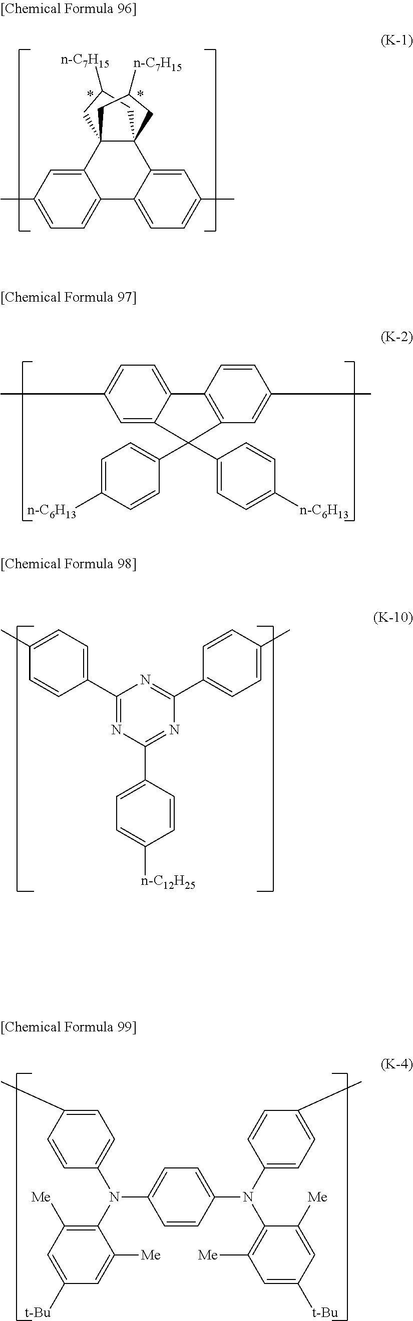

Under an argon atmosphere, Compound 4 synthesized in Example 1 (0.492 g, 0.80 mmol), the compound (1.477 g, 2.00 mmol) represented by the following formula (M-2-E):

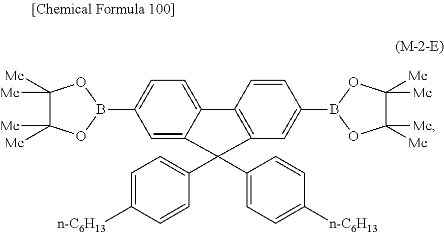

the compound (0.258 g, 0.40 mmol) represented by the following formula (M-2-BR):

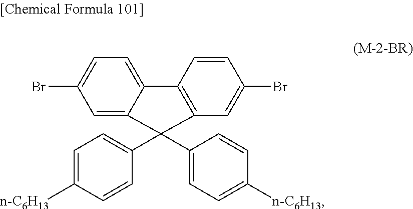

the compound (0.381 g, 0.60 mmol) represented by the following formula (M-10-BR):

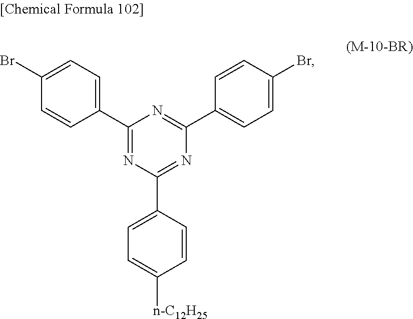

the compound (0.148 g, 0.20 mmol) represented by the following formula (M-4-BR):

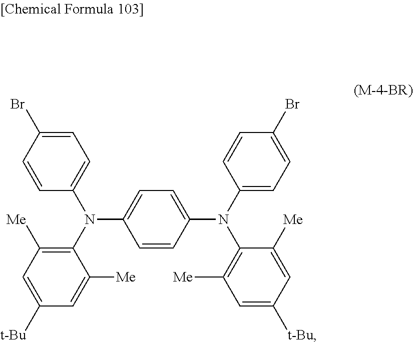 [SMILES: Cc1cc(C(C)(C)C)cc(C)c1N(c1ccc(Br)cc1)c1ccc(N(c2ccc(Br)cc2)c2c(C)cc(C(C)(C)C)cc2C)cc1]

dichlorobis(tris(o-methoxyphenyl))phosphinepalladium (1.77 mg), and toluene (50 mL) were mixed, and heated at 105° C.

A 20% by weight tetraethylammonium hydroxide aqueous solution (6.6 mL) was dropped into a reaction solution, and refluxing was performed for 3 hours. After the reaction, phenylboronic acid (24 mg) and toluene (5 mL) were added to the reaction solution, and refluxing was further performed for 18 hours. Next, a sodium diethyldithiocarbamate aqueous solution was added to the reaction solution, and stirring was performed at 80° C. for 2 hours. The obtained mixture was cooled, and an organic layer was washed twice with water, twice with a 3% by weight acetic acid aqueous solution, and twice with water. The obtained solution was dropped into methanol; then, a precipitate was produced, and filtered out to obtain a precipitate. The precipitate was dissolved in toluene, and the solution was passed through an alumina column and a silica gel column sequentially; thereby, the solution was refined. The obtained solution was dropped into methanol, and stirred; the obtained precipitate was filtered out, and dried to obtain 1.39 g of Polymer Compound A5. The polystyrene-equivalent number-average molecular weight of Polymer Compound A5 was 0.90×10⁵, and the polystyrene-equivalent weight-average molecular weight thereof was 2.29×10⁵.

### Synthesis Example 2

### Synthesis of Polymer Compound AA

Synthesis of a polymer (Polymer Compound AA) having the constitutional unit represented by the following formula (K-9), the constitutional unit represented by the following formula (K-10), and the constitutional unit represented by the following formula (K-3) at a molar ratio of 42:8:50 (a theoretical value based on prepared raw materials) was performed as follows.

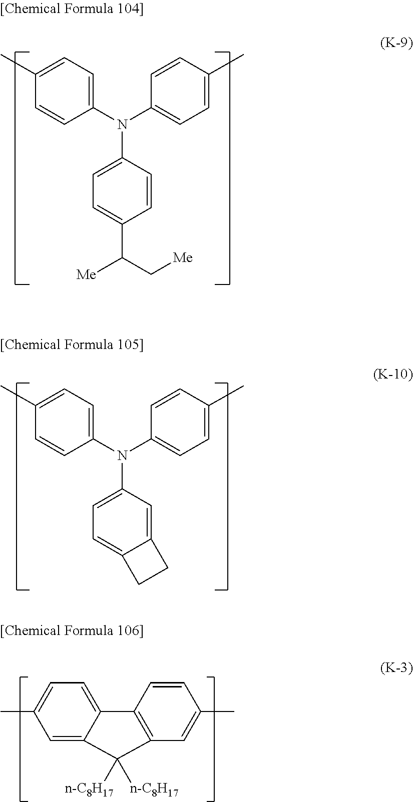

Under an argon atmosphere, the compound (17.57 g, 33.13 mmol) represented by the following formula (M-3-Z):

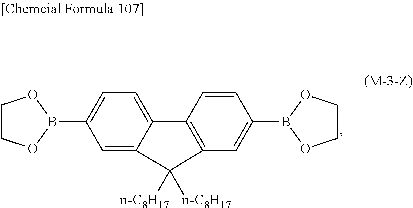

the compound (12.88 g, 28.05 mmol) represented by the following formula (M-9-BR):

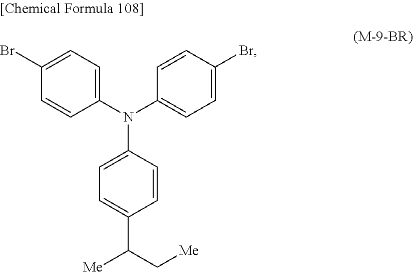 [SMILES: CCC(C)c1ccc(N(c2ccc(Br)cc2)c2ccc(Br)cc2)cc1]

the compound (2.15 mg, 5.01 mmol) represented by the following formula (M-10-BR):

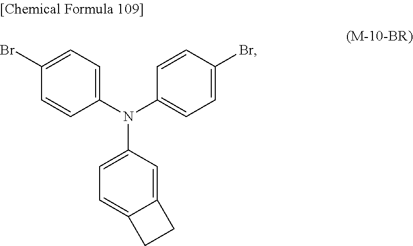 [SMILES: Brc1ccc(N(c2ccc(Br)cc2)c2ccc3c(c2)CC3)cc1]

palladium(II) acetate (7.4 mg), tris(2-methylphenyl)phosphine (70 mg), a 0.74M toluene solution of quaternary ammonium chloride (Aliquat (registered trademark 336, made by Sigma-Aldrich Corporation, 3 g), and toluene (200 g) were mixed.

A 18% by weight sodium carbonate aqueous solution (64 g) was dropped into the mixed liquid; the mixed liquid was heated for 3 hours or more and refluxed. After the reaction, phenylboronic acid (0.4 g) was added to the mixed liquid, and refluxing was further performed for

5 hours or more. Next, the reaction solution was diluted with toluene, and washed with a 3% by weight acetic acid aqueous solution and ion exchange water in this order; then, sodium diethyldithiocarbamate trihydrate (1.5 g) was added to the extracted organic layer, and stirred for 4 hours. The obtained solution was refined by column chromatography using an equivalent mixture of alumina and silica gel as a stationary phase. The obtained toluene solution was dropped into methanol, and stirred; the obtained precipitate was filtered out, and dried to obtain Polymer Compound AA. The polystyrene-equivalent number-average molecular weight of Polymer Compound AA was 8.9×10⁴, and the polystyrene-equivalent weight-average molecular weight thereof was 4.2×10⁵.

### Example 8

### Production and Evaluation of Light-Emitting Device 1

A film having a thickness of 35 nm was formed using a ethylene glycol monobutyl ether/water=3/2 (volume ratio) mixed liquid of polythiophenesulfonic acid (Sigma-Aldrich Corporation, trade name: Plexcore OC 1200) by spin coating on a glass substrate on which an ITO film having a thickness of 45 nm was formed by a sputtering method, and dried on a hot plate at 170° C. for 15 minutes.

Next, Polymer Compound AA was dissolved in xylene to prepare a 0.7% by weight xylene solution. By spin coating using the xylene solution, a film having a thickness of 20 nm was formed. This was heated on the hot plate in a nitrogen gas atmosphere at 180° C. for 60 minutes.

Next, Polymer Compound A1 was dissolved in xylene to prepare a 1.3% by weight xylene solution. By spin coating using the xylene solution, a film having a thickness of 65 nm was formed; the film was dried by heating in the nitrogen atmosphere at 130° C. for 10 hours; then, as the cathode, approximately 3 nm of sodium fluoride, and then approximately 80 nm of aluminum were vapor deposited to produce Light-emitting device 1. The vapor deposition of the metal was started after the degree of vacuum reached 1×10⁴ Pa or less.

Voltage was applied to the obtained Light-emitting device 1; EL light emission having a peak at 455 nm was obtained from the device, and the maximum light emission efficiency was 8.8 cd/A. The results are shown in Table 1.

### Example 9

### Production and Evaluation of Light-Emitting Device 2

Light-emitting device 2 was produced in the same manner as in Example 8 except that Polymer Compound A2 was used instead of Polymer Compound A1 in Example 8. Voltage was applied to the obtained Light-emitting device 2; EL light emission having a peak at 460 nm was obtained from the device, and the maximum light emission efficiency was 9.0 cd/A. The results are shown in Table 1.

### Example 10

### Production and Evaluation of Light-Emitting Device 3

Light-emitting device 3 was produced in the same manner as in Example 8 except that Polymer Compound A3 was used instead of Polymer Compound A1 in Example 8. Voltage was applied to the obtained Light-emitting device 3; EL light emission having a peak at 460 nm was obtained from the device, and the maximum light emission efficiency was 8.8 cd/A. The results are shown in Table 1.

### Example 11

### Production and Evaluation of Light-Emitting Device 4

Light-emitting device 4 was produced in the same manner as in Example 8 except that Polymer Compound A4 was used instead of Polymer Compound A1 in Example 8. Voltage was applied to the obtained Light-emitting device 4; EL light emission having a peak at 445 nm was obtained from the device, and the maximum light emission efficiency was 5.1 cd/A. The results are shown in Table 1.

### Comparative Example 1

### Synthesis of Polymer Compound B, and Production and Evaluation of Light-Emitting Device C1

Synthesis of a polymer (Polymer Compound B) having the constitutional unit represented by the following formula (K-1), the constitutional unit represented by the following formula (K-2), and the constitutional unit represented by the following formula (K-3) at a molar ratio of 20:50:30 (a theoretical value based on prepared raw materials) was performed as follows.

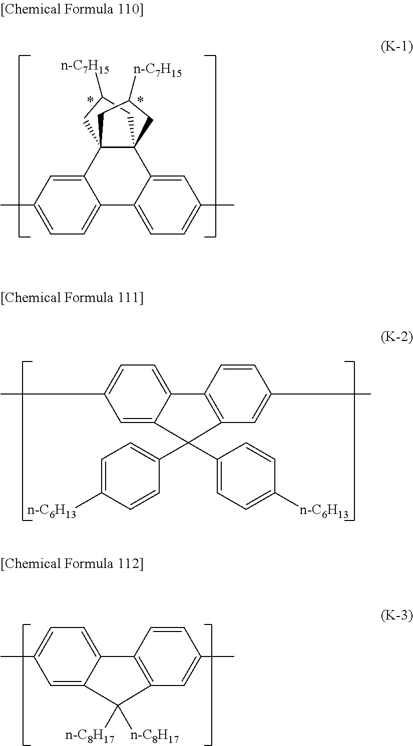

Under an argon atmosphere, Compound 4 synthesized in Example 1 (0.492 g, 0.80 mmol), the compound (1.477 g, 2.00 mmol) represented by the following formula (M-2-E):

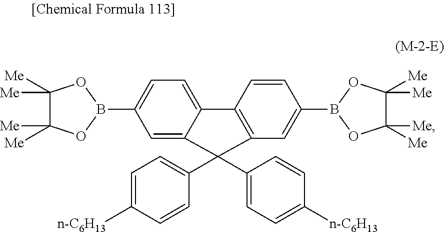

the compound (0.658 g, 1.20 mmol) represented by the following formula (M-3-BR):

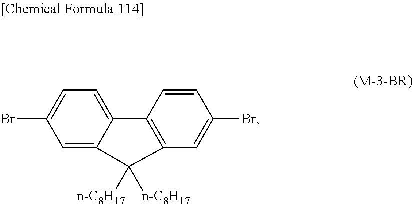

dichlorobis(tris(o-methoxyphenyl))phosphinepalladium (1.77 mg), and toluene (50 mL) were mixed, and heated at 105° C.

A 20% by weight tetraethylammonium hydroxide aqueous solution (6.6 mL) was dropped into the reaction solution, and refluxing was performed for 2 hours 40 minutes. After the reaction, phenylboronic acid (24 mg), and toluene (5 mL) were added to the reaction solution, and refluxing was further performed for 18.5 hours. Next, a sodium diethyldithiocarbamate aqueous solution was added to the reaction solution, and stirring was performed at 80° C. for 2 hours.

The obtained mixture was cooled, and an organic layer was washed twice with water, twice with a 3% by weight acetic acid aqueous solution, and twice with water. The obtained solution was dropped into methanol; then, a precipitate was produced, and filtered out to obtain a precipitate. The precipitate was dissolved in toluene, and the solution was passed through an alumina column and a silica gel column sequentially; thereby, the solution was refined. The obtained solution was dropped into methanol, and stirred; the obtained precipitate was filtered out, and dried to obtain 1.31 g of Polymer Compound B. The polystyrene-equivalent number-average molecular weight of Polymer Compound B was 9.6×10⁴, and the polystyrene-equivalent weight-average molecular weight thereof was 2.44×10⁵.

Light-emitting device C1 was produced in the same manner as in Example 8 except that Polymer Compound B was used instead of Polymer Compound A1 in Example 8. Voltage was applied to the obtained Light-emitting device C1; EL light emission having a peak at 435 nm was obtained from the device, and the maximum light emission efficiency was 4.1 cd/A. The results are shown in Table 1.

### Comparative Example 2

### Synthesis of Polymer Compound C, and, Production and Evaluation of Light-Emitting Device C2

Synthesis of a polymer (Polymer Compound C) having the constitutional unit represented by the following formula (K-2), the constitutional unit represented by the following formula (K-3), and the constitutional unit represented by the following formula (K-4) at a molar ratio of 50:45:5 (a theoretical value based on prepared raw materials) was performed as follows.

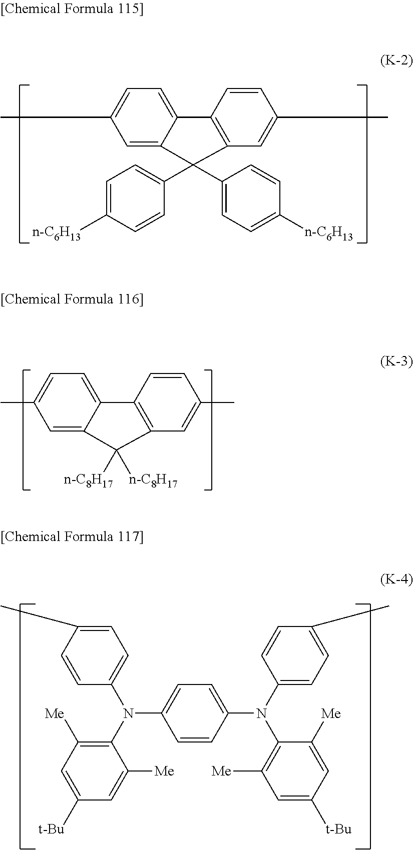

Under an argon atmosphere, the compound (4.3884 g, 5.94 mmol) represented by the following formula (M-2-E):

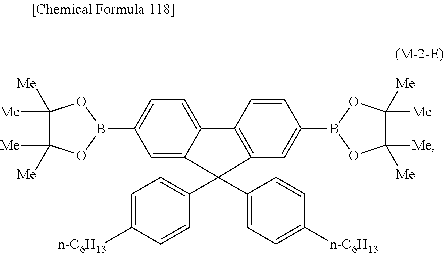

the compound (2.9621 g, 5.40 mmol) represented by the following formula (M-3-BR):

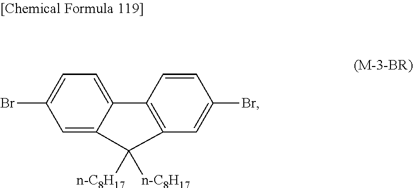

the compound (0.4430 g, 0.60 mmol) represented by the following formula (M-4-BR):

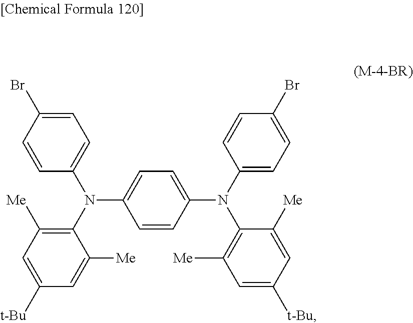 [SMILES: Cc1cc(C(C)(C)C)cc(C)c1N(c1ccc(Br)cc1)c1ccc(N(c2ccc(Br)cc2)c2c(C)cc(C(C)(C)C)cc2C)cc1]

palladium acetate (3.24 mg), tris(o-methoxyphenyl)phosphine (19.3 mg), and toluene (67 mL) were mixed, and heated to 105° C.

A 20% by weight tetraethylammonium hydroxide aqueous solution (20 mL) was dropped into the reaction solution, and refluxing was performed for 2 hours. After the reaction, phenylboronic acid (370 mg) was added to the reaction solution, and refluxing was further performed for 2 hours. Next, a sodium diethyldithiocarbamate aqueous solution was added to the reaction solution, and stirring was performed at 80° C. for 2 hours. The obtained mixture was cooled, and washed twice with water, twice with a 3% by weight acetic acid aqueous solution, and twice with water. The obtained solution was dropped into methanol, and filtration was performed to obtain a precipitate. The precipitate was dissolved in toluene, and the solution was passed through an alumina column and a silica gel column sequentially; thereby, the solution was refined. The obtained solution was dropped into methanol, and stirred; the obtained precipitate was filtered out, and dried to obtain 3.49 g of Polymer Compound C. The polystyrene-equivalent number-average molecular weight of Polymer Compound C was 1.5×10⁵, and the polystyrene-equivalent weight-average molecular weight thereof was 3.8×10⁵.

Light-emitting device C2 was produced in the same manner as in Example 8 except that Polymer Compound C was used instead of Polymer Compound A1 in Example 8. Voltage was applied to the obtained Light-emitting device C2; EL light emission having a peak at 455 nm was obtained from the device, and the maximum light emission efficiency was 7.6 cd/A. The results are shown in Table 1.

### Comparative Example 3

### Synthesis of Polymer Compound D, and Production and Evaluation of Light-Emitting Device C3

Synthesis of a polymer (Polymer Compound D) having the constitutional unit represented by the following formula (K-2), the constitutional unit represented by the following formula (K-3), the constitutional unit represented by the following formula (K-11), and the constitutional unit represented by the following formula (K-4) at a molar ratio of 50:25:20:5 (a theoretical value based on prepared raw materials) was performed as follows.

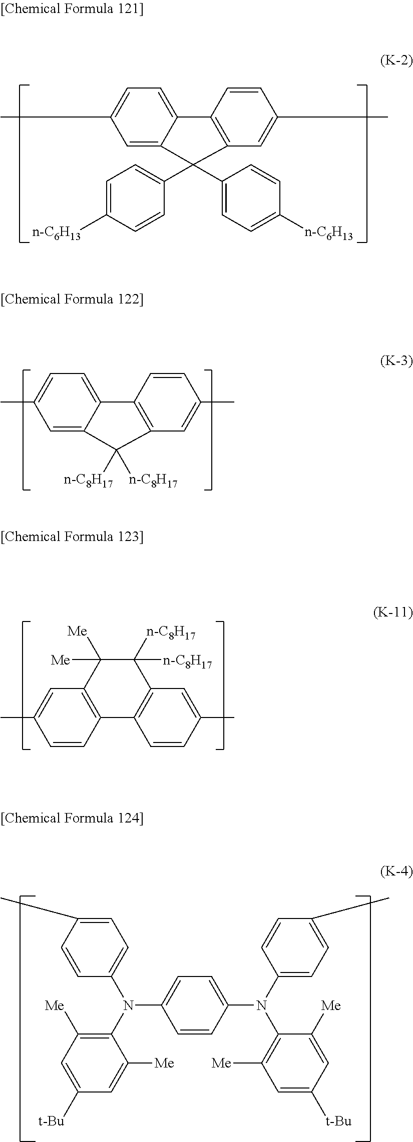

Under an argon atmosphere, the compound (1.477 g, 2.00 mmol) represented by the following formula (M-2-E):

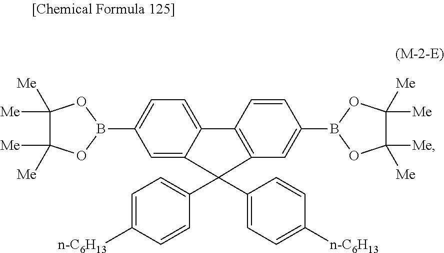

the compound (0.548 g, 1.00 mmol) represented by the following formula (M-3-BR):

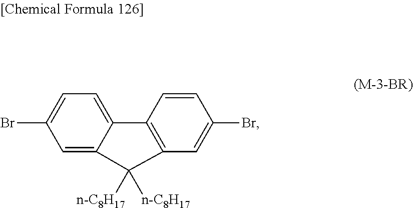

the compound (0.472 g, 0.80 mmol) represented by the following formula (M-11-BR):

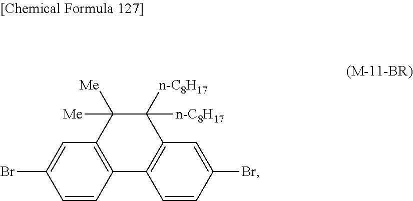

the compound (0.148 g, 0.20 mmol) represented by the following formula (M-4-BR):

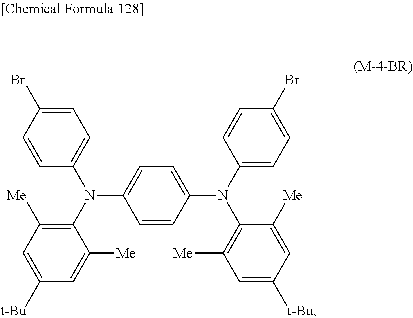 [SMILES: Cc1cc(C(C)(C)C)cc(C)c1N(c1ccc(Br)cc1)c1ccc(N(c2ccc(Br)cc2)c2c(C)cc(C(C)(C)C)cc2C)cc1]

dichlorobis(tris(o-methoxyphenyl))phosphinepalladium (1.77 mg), and toluene (50 mL) were mixed, and heated at 105° C.

A 20% by weight tetraethylammonium hydroxide aqueous solution (6.6 mL) was dropped into a reaction solution, and refluxing was performed for 3 hours. After the reaction, phenylboronic acid (24 mg) and toluene (5 mL) were added to the reaction solution, and refluxing was further performed for 18 hours. Next, a sodium diethyldithiocarbamate aqueous solution was added to the reaction solution, and stirring was performed at 80° C. for 2 hours. The obtained mixture was cooled, and an organic layer was washed twice with water, twice with a 3% by weight acetic acid aqueous solution, and twice with water. The obtained solution was dropped into methanol; then, a precipitate was produced, and filtered out to obtain a precipitate. The precipitate was dissolved in toluene, and the solution was passed through an alumina column and a silica gel column sequentially; thereby, the solution was refined. The obtained solution was dropped into methanol, and stirred; the obtained precipitate was filtered out, and dried to obtain 1.31 g of Polymer Compound D. The polystyrene-equivalent number-average molecular weight of Polymer Compound D was 0.91×10⁵, and the polystyrene-equivalent weight-average molecular weight thereof was 2.47×10⁵.

Light-emitting device C3 was produced in the same manner as in Example 8 except that Polymer Compound D was used instead of Polymer Compound A1 in Example 8. Voltage was applied to the obtained Light-emitting device C3; EL light emission having a peak at 460 nm was obtained from the device, and the maximum light emission efficiency was 7.5 cd/A. The results are shown in Table 1.

TABLE 1

| | Light- | | The maximum | Light emission |
|---|---|---|---|---|
| | emitting | Polymer | light emission | peak wavelength |
| | device | compound | efficiency (cd/A) | (nm) |
| Example 8 | 1 | A1 | 8.8 | 455 |
| Example 9 | 2 | A2 | 9.0 | 460 |
| Example 10 | 3 | A3 | 8.8 | 460 |
| Example 11 | 4 | A4 | 5.1 | 445 |
| Comparative | C1 | B | 4.1 | 435 |
| Example 1 | | | | |
| Comparative | C2 | C | 7.6 | 455 |
| Example 2 | | | | |
| Comparative | C3 | D | 7.5 | 460 |
| Example 3 | | | | |

### REFERENCE SIGNS LIST

10 . . . substrate, 11 . . . anode, 12 . . . hole injection layer, 13 . . . hole transport layer, 14 . . . light-emitting layer, 15 . . . electron transport layer, 16 . . . electron injection layer, 17 . . . cathode, 20 . . . substrate, 21 . . . anode, 22 . . . hole injection layer, 23 . . . light-emitting layer, 24 . . . cathode, 25 . . . protective layer, 100 . . . light-emitting device, 110 . . . light-emitting device, 200 . . . surface light source.

1. A polymer compound having a constitutional unit represented by the following formula (1) and a constitutional unit represented by the following formula (2): 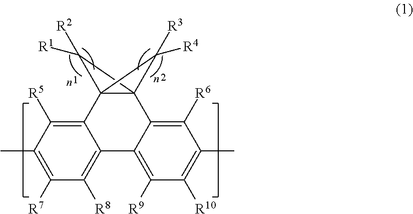
wherein n¹ and n² each independently represent an integer of 1 to 5;
R¹, R², R³, R⁴, R⁵, R⁶, R⁷, R⁸, R⁹, and R¹⁰ each independently represent a hydrogen atom, an unsubstituted or substituted alkyl group, an unsubstituted or substituted alkoxy group, an unsubstituted or substituted aryl group, an unsubstituted or substituted aryloxy group, or an unsubstituted or substituted monovalent heterocyclic group;
when R¹, R², R³, and R⁴ exist in plural, the plurality of R¹, R², R³, or R⁴ may be the same or different from each other;
among R¹, R², R³, and R⁴, adjacent groups may be linked to each other to form a cyclic structure; and among R⁷, R⁸, R⁹, and R¹⁰, adjacent groups may be linked to each other to form a cyclic structure; 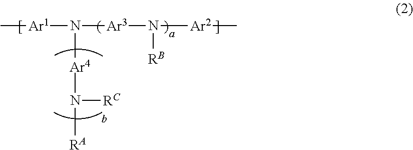
wherein a and b each independently represent 0 or 1;
Ar¹, Ar², Ar³, and Ar⁴ each independently represent an unsubstituted or substituted arylene group, an unsubstituted or substituted divalent heterocyclic group, or a divalent group in which two or more same or different groups selected from arylene groups and divalent heterocyclic groups are linked (the group may have a substituent);
R^{A}, R^{B}, and R^{C} each independently represent a hydrogen atom, an unsubstituted or substituted alkyl group, an unsubstituted or substituted aryl group, or an unsubstituted or substituted monovalent heterocyclic group; and
Ar¹, Ar², Ar³, and Ar⁴ each may be bonded to a group other than the group to form a cyclic structure, the other group being bonded to a nitrogen atom to which the group is bonded.

2. The polymer compound according to claim 1, wherein at least one of the constitutional units represented by the formula (2) is a constitutional unit represented by the following formula (3): 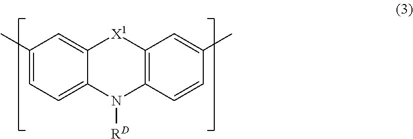
wherein RD represents a hydrogen atom, an unsubstituted or substituted alkyl group, an unsubstituted or substituted aryl group, or an unsubstituted or substituted monovalent heterocyclic group; and
X¹ represents a single bond, an oxygen atom, a sulfur atom, or a group represented by —C(R¹¹)₂— (R¹¹ represents an unsubstituted or substituted alkyl group or an unsubstituted or substituted aryl group; and a plurality of R¹¹ present may be the same or different from each other).

3. The polymer compound according to claim 1, further having a constitutional unit represented by the following formula (4): 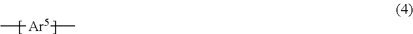
wherein Ar⁵ represents an unsubstituted or substituted arylene group, an unsubstituted or substituted divalent heterocyclic group, or a divalent group in which two or more same or different groups selected from arylene groups and divalent heterocyclic groups are linked (the group may have a substituent); and the constitutional unit represented by the formula (4) is different from the constitutional unit represented by the formula (1).

4. The polymer compound according to claim 3, wherein at least one of the constitutional units represented by the formula (4) is a constitutional unit consisting of an unsubstituted or substituted fluorenediyl group.

5. The polymer compound according to claim 4, wherein at least one of the constitutional units represented by the formula (4) is a constitutional unit consisting of an unsubstituted or substituted 2,7-fluorenediyl group.

6. The polymer compound according to claim 3, wherein at least one of the constitutional units represented by the formula (4) is a constitutional unit consisting of the group selected from the group consisting of an unsubstituted or substituted phenylene group, an unsubstituted or substituted naphthalenediyl group, an unsubstituted or substituted anthracenediyl group, and groups represented by the following formula (5′): 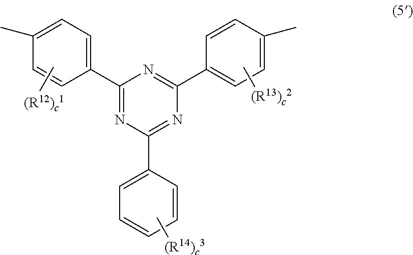
wherein c¹ and c² each independently represent an integer of 0 to 4; c³ represents an integer of 0 to 5;
R¹², R¹³, and R¹⁴ each independently represent an unsubstituted or substituted alkyl group, an unsubstituted or substituted alkoxy group, an unsubstituted or substituted aryl group, an unsubstituted or substituted aryloxy group, an unsubstituted or substituted monovalent heterocyclic group, an unsubstituted or substituted alkoxycarbonyl group, an unsubstituted or substituted silyl group, a halogen atom, a carboxyl group, or a cyano group; and
when R¹², R¹³, and R¹⁴ exist in plural, the plurality of R¹², R¹³, or R¹⁴ may be the same or different from each other.

7. The polymer compound according to claim 6 having the constitutional unit represented by the formula (1), the constitutional unit represented by the formula (2), a constitutional unit consisting of an unsubstituted or substituted fluorenediyl group, and a constitutional unit consisting of an unsubstituted or substituted phenylene group.

8. The polymer compound according to claim 6 having the constitutional unit represented by the formula (1), the constitutional unit represented by the formula (2), a constitutional unit consisting of an unsubstituted or substituted fluorenediyl group, and a constitutional unit consisting of an unsubstituted or substituted naphthalenediyl group.

9. The polymer compound according to claim 6 having the constitutional unit represented by the formula (1), the constitutional unit represented by the formula (2), a constitutional unit consisting of an unsubstituted or substituted fluorenediyl group, and a constitutional unit consisting of an unsubstituted or substituted anthracenediyl group.

10. The polymer compound according to claim 6 having the constitutional unit represented by the formula (1), the constitutional unit represented by the formula (2), a constitutional unit consisting of an unsubstituted or substituted fluorenediyl group, and a constitutional unit represented by the following formula (5): 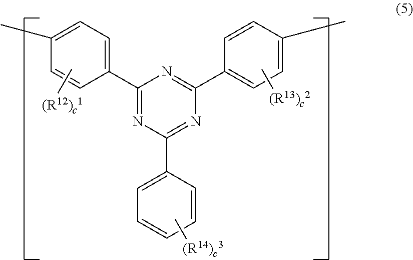
wherein c¹ and c² each independently represent an integer of 0 to 4; c³ represents an integer of 0 to 5;
R¹², R¹³, and R¹⁴ each independently represent an unsubstituted or substituted alkyl group, an unsubstituted or substituted alkoxy group, an unsubstituted or substituted aryl group, an unsubstituted or substituted aryloxy group, an unsubstituted or substituted monovalent heterocyclic group, an unsubstituted or substituted alkoxycarbonyl group, an unsubstituted or substituted silyl group, a halogen atom, a carboxyl group, or a cyano group; and
when R¹², R¹³, and R¹⁴ exist in plural, the plurality of R¹², R¹³, or R¹⁴ may be the same or different from each other.

11. The polymer compound according to claim 1, wherein n¹ and n² in the formula (1) each independently represent 3 or 4.

12. A compound represented by the following formula (6): 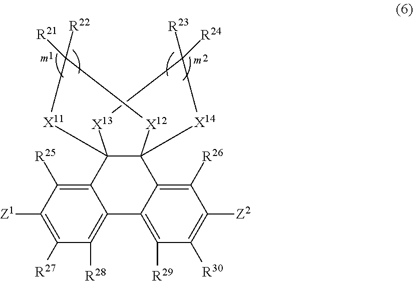
wherein m¹ and m² each independently represent 1 or 2;
R²¹, R²², R²³, R²⁴, R²⁵, R²⁶, R²⁷, R²⁸, R²⁹, and R³⁰ each independently represent a hydrogen atom, an unsubstituted or substituted alkyl group, an unsubstituted or substituted alkoxy group, an unsubstituted or substituted aryl group, an unsubstituted or substituted aryloxy group, or an unsubstituted or substituted monovalent heterocyclic group;
X¹¹, X¹², X¹³, and X¹⁴ each independently represent a group represented by —C(R³¹)₂—(R³¹ represents a hydrogen atom, an unsubstituted or substituted alkyl group, an unsubstituted or substituted alkoxy group, an unsubstituted or substituted aryl group, an unsubstituted or substituted aryloxy group, or an unsubstituted or substituted monovalent heterocyclic group; and a plurality of R³¹ present may be the same or different from each other);
among R²⁷, R²⁸, R²⁹, and R³⁰, adjacent groups may be linked to each other to form a cyclic structure; and
Z¹ and Z² each independently represent a group selected from the following substituent group;
with the proviso that among R²¹, R²², R²³, and R²⁴, at least one is a group other than a hydrogen atom;
<substituent group>
a chlorine atom, a bromine atom, an iodine atom,
a group represented by —O—S(═O)₂R⁴¹ wherein R⁴¹ represents an alkyl group, or an aryl group that may be substituted with an alkyl group, an alkoxy group, a nitro group, a fluorine atom, or a cyano group,
a group represented by —B(OR⁴²)₂ wherein R⁴² represents a hydrogen atom or an alkyl group; and a plurality of R⁴² present may be the same or different from each other and may be linked to each other to form a cyclic structure,
a group represented by —BF₄Q¹ wherein Q¹ represents a monovalent cation selected from the group consisting of Li⁺, Na⁺, K⁺, Rb⁺, and Cs⁺,
a group represented by —MgY¹ wherein Y¹ represents a chlorine atom, a bromine atom, or an iodine atom,
a group represented by —ZnY² wherein Y² represents a chlorine atom, a bromine atom, or an iodine atom, and
a group represented by —Sn(R⁴³)₃ wherein R⁴³ represents a hydrogen atom or an alkyl group; and a plurality of R⁴³ present may be the same or different from each other and may be linked to each other to form a cyclic structure.

13. A composition comprising the polymer compound according to claim 1, and at least one selected from the group consisting of a hole transport material, an electron transport material, and a light-emitting material.

14. A liquid composition comprising the polymer compound according to claim 1, and a solvent.

15. An organic film comprising the polymer compound according to claim 1.

16. An organic film prepared using the composition according to claim 13.

17. A light-emitting device having the organic film according to claim 15.

18. A surface light source having the light-emitting device according to claim 17.

19. A display device having the light-emitting device according to claim 17.
